(19) United States
(12) Patent Application Publication
(10) Pub. No.: US 2023/0210771 A1
(43) Pub. Date: Jul. 6, 2023

(54) COMPOSITIONS FOR THE DELIVERY OF THERAPEUTIC AGENTS AND METHODS OF USE AND MAKING THEREOF

(71) Applicant: DISRUPTION LABS INC., Austin, TX (US)

(72) Inventors: Brian R. SLOAT, Austin, TX (US); Michael A SANDOVAL, Austin, TX (US)

(73) Assignee: DISRUPTION LABS INC., Austin, TX (US)

(21) Appl. No.: 18/001,089

(22) PCT Filed: Jun. 8, 2021
(86) PCT No.: PCT/US2021/036393

§ 371 Date: Dec. 8, 2022

Related U.S. Application Data
(60) Provisional application No. 63/037,321, filed on Jun. 10, 2020.
(60) Provisional application No. 63/037,997, filed on Jun. 11, 2020.

(51) Int. Cl. A61K 9/127; A61K 31/05; A61K 45/06; A61K 31/015; A61K 47/28; A61K 47/24; A61K 47/14

(52) U.S. Cl. CPC A61K 9/127; A61K 31/05; A61K 45/06; A61K 31/015; A61K 47/28; A61K 47/24; A61K 47/14; B82Y 5/00

(57) ABSTRACT

Some embodiments pertain to nanoparticle-based compositions and their use in methods for the delivery of therapeutic agents to subjects. In some embodiments, the compositions are stable for prolonged periods of time and provide enhanced bioavailability.

### CROSS REFERENCE TO RELATED APPLICATIONS

This application claims the benefit of priority to U.S. Provisional Application No. 63/037,997, filed Jun. 11, 2020, and U.S. Provisional Application No. 63/037,321, filed Jun. 10, 2020, the disclosures of each of which are hereby incorporated by reference in their entireties. Any and all applications for which a priority claim is identified in the Application Data Sheet as filed with the present application are also hereby incorporated by reference in their entireties under 37 CFR 1.57.

### FIELD

This disclosure relates generally to lipid, nanoparticle-based compositions (e.g., liposomal, solid lipid particles, oil-in-water emulsions, etc.) and their use in methods for the delivery of hydrophobic and/or hydrophilic therapeutic agents (e.g., vitamins, nutrients, plant extracts, nutraceuticals, pharmaceuticals, or other beneficial agents for delivery) to subjects. In some embodiments, the lipid compositions comprise a cannabinoid or combinations of cannabinoids (e.g., such as cannabidiol (“CBD”) and other cannabinoids) as a therapeutic agent(s). In some embodiments, the compositions are stable (e.g., at room temperature) for prolonged periods of time.

### BACKGROUND

### Description of the Related Art

Therapeutic agents such as CBD can be used for alleviating pain (e.g., from multiple sclerosis), treating epilepsy, and the treatment of certain neurological disorders. Therapeutic agents can be taken into the body in multiple different ways, including by inhalation of cannabis smoke or vapor, as an aerosol spray into the cheek, and by mouth. Therapeutic agents may be supplied as an oil (e.g., CBD-dominant hemp extract oil), capsules, dried, or as a prescription liquid solution.

### SUMMARY

Some embodiments disclosed herein pertain to a particle composition and/or a lipid-based particle composition for the delivery of an active agent (e.g., a therapeutic agent). In some embodiments, the particle is a lipid particle. In some embodiments, the particle is a nanoscale particle. In some embodiments, the particle is a microscale particle. In some embodiments, the particle is liposomal (e.g., is a liposome). In some embodiments, the particle comprises, consists of, or consists essentially of one or more of a phospholipid component, a non-phospholipid lipid component (e.g., a medium and/or long chain triglyceride component), a sterol component, and/or water. In some embodiments, the particle comprises, consists of, or consists essentially of one or more of a phospholipid component, a non-phospholipid lipid component, a sterol component, water, and/or an therapeutic agent. In some embodiments, the particle is liposomal (e.g., is a liposome). In some embodiments, the particle comprises, consists of, or consists essentially of one or more of a phospholipid component, a non-phospholipid lipid component (e.g., a medium and/or long chain triglyceride component), and/or a sterol component. In some embodiments, the particle comprises, consists of, or consists essentially of one or more of a phospholipid component, a non-phospholipid lipid component, a sterol component, and/or an therapeutic agent. In some embodiments, the particle comprises an active ingredient (e.g., a therapeutic agent) and/or a combination of active ingredients (e.g., therapeutic agents).

In some embodiments, the active ingredient or combination of active ingredients comprise a mixture of therapeutics isolated from a plant source (e.g., a full spectrum mixture). In some embodiments, the active ingredient or combination of active ingredients comprise one or more cannabinoids. In some embodiments, the one or more cannabinoids include one or more phytocannabinoids (e.g., combinations of phytocannabinoids). In some embodiments, the one or more phytocannabinoids comprise CBD. In some embodiments, the lipid constituents of the particle allow the particle to solubilize CBD (or other phytocannabinoids or cannabinoids) of high purity. In some embodiments, the CBD in the particle is of sufficient purity to provide a crystalline and/or solid (e.g., an amorphous or crystalline powder). In some embodiments, the CBD not an oil. In some embodiments, the active ingredient or combination of active ingredients comprise one or more non-cannabinoid therapeutic agents. In some embodiments, the therapeutic agent comprises, consists of, or consists essentially of a synthetic therapeutic agent, non-synthetic therapeutic agent, and/or combinations thereof.

In some embodiments, the phytocannabinoid of a lipid-based particle composition as disclosed herein is a single phytocannabinoid (e.g., CBD). In some embodiments, the phytocannabinoid (e.g., CBD) has a purity by weight % of equal to or greater than about: 70%, 80%, 85%, 90%, 95%, 97%, 98%, 99%, 100%, or ranges including and/or spanning the aforementioned values. In some embodiments, the phytocannabinoid (e.g., CBD) is present in the lipid-based particle composition at dry weight % of equal to or greater than about: 5%, 8%, 10%, 15%, 20%, or ranges including and/or spanning the aforementioned values. In some embodiments, the phytocannabinoid is free of or essentially free of THC. In some embodiments, the phytocannabinoid (e.g., CBD) has a THC content by weight % of equal to or less than about: 1%, 0.5%, 0.25%, 0.1%, 0%, or ranges including and/or spanning the aforementioned values. In some embodiments, where present, THC is present below the limit of quantitation (LOQ) (e.g., when analyzed by high pressure liquid chromatography (HPLC) with standard detectors, such as UV/Vis, photodiode array, refractive index, fluorescence, light scattering, conductivity, and the like).

In some embodiments, the phospholipid component comprises, consists of, or consists essentially of one or more of phosphatidic acid, phosphatidylethanolamine, phosphatidylcholine, phosphatidylserine, phosphatidylinositol, phosphatidylinositol phosphate, phosphatidylinositol bisphosphate, and phosphatidylinositol trisphosphate. In some embodiments, the phospholipid component comprises phosphatidylcholine. In some embodiments, the phospholipid component is a single phospholipid. In some embodiments, the phospholipid component is phosphatidylcholine. In some embodiments, the phosphatidylcholine is highly pure. In some embodiments, the phosphatidylcholine has a purity by weight % of equal to or greater than about: 97%, 98%, 99%, 100%, or ranges including and/or spanning the aforementioned values. In some embodiments, the phosphatidylcholine is present in the lipid-based particle composition at dry weight % of equal to or greater than about: 10%, 20%, 30,%, 35%, 40%, 45%, 50%, or ranges including and/or spanning the aforementioned values. In some embodiments, the phospholipid component comprises, consists of, or consists essentially of a synthetic phospholipid, non-synthetic phospholipid, and/or combinations thereof.

In some embodiments, where present, the lipid component (e.g., the lipid component that is not a phospholipid) comprises, consists of, or consists essentially of a triglyceride. In some embodiments, where present, the lipid component (e.g., the lipid component that is not a phospholipid) comprises, consists of, or consists essentially of a fatty acid (or fatty acids). In some embodiments, the lipid component comprises a medium chain triglyceride (MCT). In some embodiments, the medium chain triglyceride comprises a fatty acid selected from one or more of caprioc acid, octanoic acid, capric acid, caprylic acid, and/or lauric acid (e.g., is formed from). In some embodiments, the medium chain triglyceride comprises a fatty acid tail 6-12 carbons in length (e.g., 6, 7, 8, 9, 10, 11, or 12). In some embodiments, the lipid component comprises a long chain triglyceride. In some embodiments, the long chain triglyceride comprises a fatty acid tail greater than 12 carbons in length (e.g., greater than or equal to 13, 14, 15, 16, 17, 18, 19, or 20 carbons in length, or ranges including and/or spanning the aforementioned values). In some embodiments, the lipid component comprises a short chain triglyceride (SCT). In some embodiments, the short chain triglyceride comprises a fatty acid tail less than 6 carbons in length (e.g., less than or equal to 5, 4, 3, 2, 1 carbons in length, or ranges including and/or spanning the aforementioned values). In some embodiments, the lipid component is a single lipid. In some embodiments, the lipid component is MCT. In some embodiments, the MCT is highly pure. In some embodiments, the lipid component (e.g., SCT, MCT, LCT, or combination thereof) has a purity by weight % of equal to or greater than about: 90%, 95%, 97%, 98%, 99%, 100%, or ranges including and/or spanning the aforementioned values. In some embodiments, the lipid component (e.g., MCT, SCT, LCT, or combination thereof) is present in the lipid-based particle composition at dry weight % of equal to or greater than about: 10%, 20%, 30%, 35%, 40%, 45%, 50%, or ranges including and/or spanning the aforementioned values. In some embodiments, the non-phospholipid lipid component comprises, consists of, or consists essentially of a synthetic non-phospholipid lipid, non-synthetic non-phospholipid lipid, and/or combinations thereof.

In some embodiments, wherein present, the sterol component comprises, consists of, or consists essentially of cholesterol. In some embodiments, where present, the sterol component comprises, consists of, or consists essentially of a single sterol. In some embodiments, the sterol component is cholesterol. In some embodiments, the cholesterol (or other sterol) is highly pure. In some embodiments, the cholesterol (or other sterol) has a purity by weight % of equal to or greater than about: 97%, 98%, 99%, 100%, or ranges including and/or spanning the aforementioned values. In some embodiments, the cholesterol (or other sterol) is present in the lipid-based particle composition at dry weight % of equal to or greater than about: 1%, 2%, 4%, 5%, 8%, or ranges including and/or spanning the aforementioned values. In some embodiments, the sterol component comprises, consists of, or consists essentially of a synthetic sterol, a non-synthetic sterol, and/or combinations thereof.

In some embodiments, the lipid-based particle composition is aqueous while in other embodiments the composition may be provided as a dry or substantially dry solid (e.g., having a water content in weight % of less than or equal to 20%, 15%, 10%, 5%, 2%, 1%, 0.5%, or ranges including and/or spanning the aforementioned values). In some embodiments, where the lipid-based particle composition is aqueous, water may be present at a wet weight percent of equal to or less than about: 70%, 75%, 77%, 80%, 85%, or ranges including and/or spanning the aforementioned values. In some embodiments of the aqueous composition, the phytocannabinoid (e.g., CBD) is present in the composition at wet weight % of equal to or greater than about: 1%, 2%, 5%, 8%, 10%, 15%, 20%, or ranges including and/or spanning the aforementioned values. In some embodiments, the phosphatidylcholine is present in the aqueous composition at wet weight % of equal to or greater than about: 5%, 10%, 15%, 20%, or ranges including and/or spanning the aforementioned values. In some embodiments, the MCT is present in the aqueous composition at wet weight % of equal to or greater than about: 5%, 10%, 15%, 20%, or ranges including and/or spanning the aforementioned values. In some embodiments, the cholesterol is present in the aqueous composition at wet weight % of equal to or greater than about: 0.5%, 1.0%, 2.0%, 3.0%, 5.0%, or ranges including and/or spanning the aforementioned values.

In some embodiments, as disclosed elsewhere herein, the particle comprises CBD, phosphatidylcholine, cholesterol, a lipid component other than a phospholipid (e.g., one or more of a medium chain triglyceride, a long chain triglyceride, and/or hemp oil), and/or water. In some embodiments, the CBD is present in an amount of less than or equal to about 25 mg/ml. In some embodiments, the phosphatidylcholine is present in an amount of less than or equal to about 100 mg/ml. In some embodiments, the cholesterol is present in an amount of less than or equal to about 25 mg/ml. In some embodiments, the medium chain triglyceride is present in an amount of less than or equal to about 100 mg/ml.

In some embodiments, the lipid-based particle composition further comprises a preservative. In some embodiments, the preservative comprises one or more of malic acid, citric acid, potassium sorbate, sodium benzoate, and Vitamin E. In some embodiments, malic acid is present in an amount of less than or equal to about 0.85 mg/ml. In some embodiments, citric acid is present in an amount of less than or equal to about 0.85 mg/ml. In some embodiments, potassium sorbate is present in an amount of less than or equal to about 1 mg/ml. In some embodiments, sodium benzoate is present in an amount of less than or equal to about 1 mg/ml. In some embodiments, the composition further comprises a flavoring agent.

Some embodiments pertain to a lipid-based particle composition, comprising: a nanoparticle comprising: cannabidiol (CBD) that is of sufficient purity that it exists in a solid and/or powdered state prior to formulation in the nanoparticle composition at a weight percent in the composition ranging from 1% to 10%; a phosphatidylcholine at a weight percent in the composition ranging from 2.5% to 15%; a sterol at a weight percent in the composition ranging from 0.5% to 5%; and a medium chain triglyceride at a weight percent in the composition ranging from 2.5% to 15%. In some embodiments, the composition comprises water at a weight percent in the composition ranging from 60% to about 80%. In some embodiments, the nanoparticles have an average size ranging from about 75 nm to about 175 nm. In some embodiments, upon storage for a period of one month, the average size of the nanoparticles changes by less than about 20%.

In some embodiments, the lipid-based particle composition is in the form of liposomes and/or an oil-in-water nano-emulsion. In some embodiments, an appreciable amount of the nanoparticle composition does not settle and/or separate from the water upon standing for a period of at least about 12 hours. In some embodiments, the composition is configured such that when concentrated to dryness to afford a powder formulation of nanoparticles, the nanoparticle powder can be reconstituted to provide the nanoparticle composition. In some embodiments, the composition has a Tmax for CBD of less than 4.5 hours. In some embodiments, upon storage for a period of one month, the average size of the nanoparticles changes by less than about 20%. In some embodiments, the polydispersity of the nanoparticles in the composition is less than or equal to 0.15. In some embodiments, upon 90 days of storage at 25° C. and 60% relative humidity, the polydispersity of the nanoparticles changes by less than or equal to 10%. In some embodiments, upon 90 days of storage at 25° C. and 60% relative humidity, the polydispersity of the nanoparticles changes by less than or equal to 0.1. In some embodiments, the composition has a shelf life of greater than 18 months at 25° C. and 60% relative humidity. In some embodiments, upon 90 days of storage at 25° C. and 60% relative humidity, the D90 of the nanoparticles changes less than or equal to 10%. In some embodiments, the composition has a concentration max (Cmax) of 80 ng/ml after an oral dose of 15 mg/kg.

Some embodiments, pertain to a lipid-based particle composition, comprising a particle comprising cannabidiol (CBD) that is of sufficient purity that it exists in a solid and/or powdered state prior to formulation in the nanoparticle composition at a weight percent in the composition ranging from 5% to 15%, a phosphatidylcholine at a weight percent in the composition ranging from 35% to 60%, a sterol at a weight percent in the composition ranging from 2.5% to 10%, and a medium chain triglyceride at a weight percent in the composition ranging from 35% to 50%. In some embodiments, the composition further comprising a preservative. In some embodiments, the preservative comprises one or more of malic acid, citric acid, potassium sorbate, sodium benzoate, and Vitamin E. In some embodiments, the sterol is cholesterol. In some embodiments, the composition further comprises a flavoring agent.

In some embodiments, the composition has a Cmax of 80 ng/ml after an oral dose of 15 mg/kg. In some embodiments, the lipid-based particle composition is provided as a dry powder. In some embodiments, the powder is configured to be reconstituted in water to provide an aqueous solution. In some embodiments, wherein, upon reconstitution, nanoparticles within the aqueous solution have an average size ranging from about 75 nm to about 175 nm.

In some embodiments, the composition further comprising a preservative. In some embodiments, the preservative comprises one or more of malic acid, citric acid, potassium sorbate, sodium benzoate, and Vitamin E. In some embodiments, the sterol is cholesterol. In some embodiments, the composition further comprises a flavoring agent.

In some embodiments, as disclosed elsewhere herein, the lipid-based particle composition is in the form and/or comprises one or more of liposomes, an oil-in-water nano-emulsion (and/or microparticle emulsion), and/or solid lipid particles. In some embodiments, when suspended in water, an appreciable amount of the particles in the composition do not settle and/or do not separate (e.g., upon visual inspection) from the water upon standing for a period of at least about 12 hours. In some embodiments, when suspended in water, the particles remain substantially homogenously distributed in the water upon standing for a period of at least about 12 hours. In some embodiments, the nanoparticles have an average size ranging from about 10 nm to about 500 nm. In some embodiments, the composition comprises nanoparticles having an average size of less than or equal to about: 10 nm, 50 nm, 100 nm, 250 nm, 500 nm, 1000 nm, or ranges including and/or spanning the aforementioned values. In some embodiments, the composition comprises microparticles having an average size of less than or equal to about: 1000 nm, 1.5 μm, 2 μm, 3 μm, 5 μm, 10 μm or ranges including and/or spanning the aforementioned values. In some embodiments, the dried powder composition comprises microparticles that form nanoparticles (as disclosed herein) when reconstituted. In some embodiments, these dried powder compositions comprise particles having an average size of less than or equal to about: 250 nm, 500 nm, 1000 nm, 1.5 μm, 2 μm, 3 μm, 5 μm, 10 μm, 50 μm, or ranges including and/or spanning the aforementioned values. In some embodiments, upon storage for a period of one month, the average size of the nanoparticles (or microparticles) increases by less than about 10%.

In some embodiments, the lipid-based particle composition is configured such that when concentrated to dryness to afford dry particles (e.g., from any one of the oil-in-water emulsion (e.g., a nanoemulsion or microemulsion), liposome solution, and/or solid lipid particle) as a powder, the dry nanoparticles can be reconstituted to provide a reconstituted particle based solution (e.g., the nanoparticle composition). In some embodiments, when reconstituted, the average size of the nanoparticles increases or decreases by less than about 15% and/or by less than about 100%. In some embodiments, to form powders, excipients (and/or additives as disclosed elsewhere herein) may be added to the liposomes, oil-in-water nano-emulsions (and/or microparticle emulsions), and/or a solid lipid particle. In some embodiments, the excipient comprises trehalose.

Some embodiments, as disclosed elsewhere herein, pertain to a method of manufacturing a lipid-based particle composition. In some embodiments, one or more phytocannabinoids (e.g., CBD) is mixed with one or more lipophilic components of the composition to provide a solution. In some embodiments, one or more lipid components (that are not phospholipids) are added. In some embodiments, one or more sterols are added. In some embodiments, one or more phospholipids are added. In some embodiments, one or more flavoring and/or preservatives are added. In some embodiments, water is added. In some embodiments, the lipophilic ingredients are combined and the hydrophilic ingredients are combined separately. In some embodiments the lipophilic ingredients are then added to the hydrophilic ingredients. In some embodiments, the solution is passed through a microfluidizer and/or a high sheer homogenizer. In some embodiments, the process affords a particle composition.

In some embodiments, a method of manufacturing the particle composition of a phytocannabinoid is disclosed. In some embodiments, the phytocannabinoid is added to solvent. In some embodiments, one or more phospholipids are added to the solvent. In some embodiments, one or more sterols are added to the solvent. In some embodiments, one or more lipids is added to the solvent. In some embodiments, the solvent is removed to provide a substantially solid product. In some embodiments, the product is mixed with water to provide an emulsion. In some embodiments, the emulsion is passed through a microfluidizer and/or a high sheer homogenizer. In some embodiments, the process affords a nanoparticle composition.

Some embodiments pertain to a method of treating a patient in need of treatment comprising administering an effective amount therapeutic agent provided as a lipid-based particle composition as disclosed herein to the patient. Some embodiments pertain to a method of treating a patient in need of treatment comprising administering an effective amount of the composition to the patient. In some embodiments, the patient in need of treatment is a patient suffering from one or more of pain, anxiety & stress, seizures, malaise, inflammation, mood disorders, and insomnia. In some embodiments, the condition is treated by administering an effective amount of a composition as disclosed herein to the patient.

In some embodiments, the Cmax is increased relative to CBD alone or comparator embodiments (e.g., CBD oil-based products) by equal to or at least about: 15%, 20%, 50%, 100%, 150%, 200%, or ranges including and/or spanning the aforementioned values. In some embodiments, the Cmax is increased (relative to an oil-based product) by equal to or at least about: 10 ng/mL, 20 ng/mL, 30 ng/mL, 40 ng/mL, 50 ng/mL, 60 ng/mL, 70 ng/mL, 80 ng/mL, 90 ng/mL, or ranges including and/or spanning the aforementioned values.

In some embodiments, the Tmax for CBD is decreased (relative to CBD alone or a CBD in oil mixture) by equal to or at least about: 15%, 20%, 50%, 100%, 150%, 200%, or ranges including and/or spanning the aforementioned values. In some embodiments, the Tmax for CBD in a disclosed embodiment is decreased (relative to CBD alone or a CBD in oil mixture) by equal to or at least about: 15 minutes, 30 minutes, 45 minutes, 1 hour, 2 hours, or ranges including and/or spanning the aforementioned values.

In some embodiments, the AUC for CBD using a disclosed embodiment is increased (relative to CBD alone or a CBD in oil mixture) by equal to or at least about: 100 ng/mL*hr, 200 ng/mL*hr, 300 ng/mL*hr, 400 ng/mL*hr, or ranges including and/or spanning the aforementioned values. In some embodiments, the AUC is improved (relative to CBD alone or a CBD in oil mixture) by equal to or at least about: 25%, 50%, 100%, 150%, 200%, or ranges including and/or spanning the aforementioned values.

### BRIEF DESCRIPTION OF THE DRAWINGS

FIG. 1 is a flow chart showing an embodiment of a method of preparing a lipid-based particle composition as disclosed herein.

FIG. 2 is a flow chart showing another embodiment of a method for preparing a lipid-based particle composition as disclosed herein.

FIG. 3 depicts the CBD concentration in an embodiment of the disclosed lipid-based particle compositions over time when stored at 25° C./60% relative humidity.

FIG. 4 depicts the particle size of an embodiment of the disclosed lipid-based particle compositions over time when stored at 25° C./60% relative humidity.

FIGS. 5A-5E depict representative images of some embodiments of lipid nanoparticles as disclosed herein.

FIG. 6 depicts resulting Z-Average Particle Size of some embodiments after 5 microfluidization passes for embodiments prepared using solvent-free methods.

FIG. 7 depicts resulting D90 Particle Size of some embodiments after 5 microfluidization passes for embodiments prepared using solvent-free methods.

FIG. 8 depicts resulting polydispersity of some embodiments after 5 microfluidization passes for embodiments prepared using solvent-free methods.

FIG. 9A-D depict the pharmacokinetic profiles of certain embodiments of CBD lipid nanoparticle solutions, CBD lipid nanoparticle powders and CBD oil-based commercial comparators. FIG. 9A shows CBD plasma concentration data for an embodiment as disclosed herein, including for a lipid nanoparticle solution and a lipid nanoparticle powder. FIG. 9B provides a comparison of the lipid nanoparticle powder of FIG. 9A compared to commercial comparators comprising CBD oil. FIG. 9C provides a comparison of the lipid nanoparticle solution of FIG. 9A compared to commercial comparators comprising CBD oil. FIG. 9D provides an expanded view of the data in FIG. 9C.

FIG. 10 depicts the Tmax for of CBD lipid nanoparticle as disclosed herein compared to and CBD oil-based commercial comparators.

FIG. 11 depicts Half-Lives (T_{1/2}) of some embodiments of CBD lipid nanoparticle solutions, powders, and oil-based commercial comparators.

FIG. 12 depicts Area Under The Curve (AUC) of some embodiments of CBD lipid nanoparticle solutions, powders, and oil-based commercial comparators.

FIG. 13 shows data for the change in CBD lipid nanoparticle particle size in some embodiments over approximately 6 months at different solution pH.

FIG. 14 shows data for the change in CBD concentration in certain embodiments of lipid nanoparticles after 7 months at different storage conditions.

FIG. 15 shows data for various passes through a microfluidizer, including an initial particle size measurements after 1 pass through 10 passes.

FIG. 16 shows data for different particles after various passes through a microfluidizer, including particles after 1 pass through 10 passes after storage for 6 months at 25° C. with 60% relative humidity.

FIG. 17A-C shows change in particle size distribution by operating pressure measured using Z average, D90 particle sizing, and polydispersity index, respectively.

FIG. 18 shows short-term stability data for various embodiments of CBD lipid nanoparticles prepared with cholesterol alternative phytosterols.

FIGS. 19A and 19B show stability data for various embodiments of CBD lipid nanoparticles in simulated gastric and simulated intestinal fluids.

FIG. 20 shows stability data for various embodiments of CBD lipid nanoparticles.

FIG. 21 shows embodiments of CBD nanoparticles in beverages and the nanoparticle size at two time points.

### DETAILED DESCRIPTION

Some embodiments disclosed herein pertain to formulations and/or lipid-based particle compositions for the delivery of therapeutic agents to subjects. In some embodiments, the lipid-based particle compositions are nanoparticle compositions. In some embodiments, the nanoparticles comprise liposomes. Some embodiments pertain to methods of use and making the composition. In some embodiments, the lipid-based particle compositions comprise one or more therapeutic agents (e.g., single therapeutic agents or combinations thereof). In some embodiments, one or more therapeutic agents may be a cannabinoid, a phytocannabinoid, a non-cannabinoid therapeutic, and/or a combination of any of the foregoing. In some embodiments, the phytocannabinoid is cannabidiol (CBD). In some embodiments, the composition is comprised of high-quality, pure, and/or high-grade ingredients (e.g., highly pure) that yield a well-characterized, reproducible delivery system (e.g., comprising lipid-based particles). In some embodiments, the lipid-based particle compositions as disclosed herein have enhanced stability (e.g., are stable for long periods of time under various conditions). In some embodiments, the composition confers water solubility to hydrophobic therapeutic agents, to combinations of hydrophobic therapeutic agents, and/or to combinations of hydrophobic and hydrophilic therapeutic agents. In some embodiments, the composition imparts apparent solubility to a compound (or compounds) that is otherwise considered practically insoluble in water (e.g., >10 liters of water needed to dissolve 1 gram of CBD) and/or a compound (or compounds) practically water insoluble according to the biopharmaceutical classification system. In some embodiments, the lipid-based particle composition comprises a liposomal and/or nano-emulsion composition of a therapeutic agent. In some embodiments, the lipid-based particle composition is configured for oral ingestion. In some embodiments, the lipid-based particle formulation is provided as a drinkable solution, such as a beverage, elixir, tonic, or the like. While some embodiments are disclosed herein in relation to CBD, it is to be understood that other therapeutic agents, nutrients, and/or combinations thereof can be employed in the delivery systems (e.g., lipid-based particle compositions) disclosed herein (e.g., cannabinoids, phytocannabinoids, fish oils, vitamin D and other lipid soluble vitamins). In some embodiments, for example, hydrophilic therapeutic agents may also be provided in the disclosed lipid-based particle compositions (e.g., alone, in combination with other hydrophilic therapeutic agents, and/or in combination with hydrophobic therapeutic agents). Advantageously, the lipid-based particle compositions disclosed herein may enhance the delivery of and/or slow or lessen the degradation of hydrophilic or hydrophobic therapeutic agents (or combinations thereof). Additionally, while some embodiments are disclosed in relation to nanoparticles (e.g., lipid-based nanoparticles), as disclosed elsewhere herein, microparticles are also envisioned.

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

As used herein, “and/or” refers to and encompasses any and all possible combinations of one or more of the associated listed items, as well as the lack of combinations when interpreted in the alternative (“or”).

The term “effective amount,” as used herein, refers to that amount of a recited compound and/or composition that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, an effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, an improvement in a condition can be a reduction in disease symptoms or manifestations (e.g., pain, anxiety & stress, seizures, malaise, inflammation, mood disorders, insomnia, etc.). Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including, but not limited to, the activity of the composition, composition, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, curing the illness, etc.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, mini-pigs (a mini-pig is a small breed of swine weighing about 35 kg as an adult), horses, cats, dog, rabbits, rodents (e.g., rats or mice), monkeys, etc. Human subjects include neonates, infants, children, juveniles, adults and geriatric subjects. The subject can be a subject “in need of” the methods disclosed herein can be a subject that is experiencing a disease state and/or is anticipated to experience a disease state, and the methods and compositions of the invention are used for therapeutic and/or prophylactic treatment.

As used herein, the term “weight percent” (or wt %, weight %, percent by weight, etc.), when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%). As used herein, the “dry weight %” (e.g., “dry wt %”, “dry weight percent”, etc.) of an ingredient is the weight percent of that ingredient in the composition where the weight of water has not been included in the calculation of the weight percent of that ingredient. A dry weight % can be calculated for a composition that does not include water or for a composition that includes water. As used herein, the “wet weight %” (e.g., “wet wt %”, “wet weight percent”, etc.) of an ingredient is the weight percent of that ingredient in a composition where the weight of water is included in the calculation of the weight percent of that ingredient. For example, the dry weight percent of component A when 5 grams of component A is added to 95 grams of component B and 100 grams of water is 5% (e.g., 5 g A/(5 g A+95 g B)×100%). Alternatively, the wet weight percent of component A when 5 grams of component A is added to 95 grams of component B and 100 grams of water is 2.5% (e.g., 5 g A/(5 g A+95 g B+100 g water)×100%).

When referring to an amount present for one or more ingredients, the term “collectively or individually” (and variations thereof) means that the amount is intended to signify that the ingredients combined may be provided in the amount disclosed, or each individual ingredient may be provided in the amount disclosed. For example, if agents A and B are referred to as collectively or individually being present in a composition at a wt % of 5%, that means that A may be at 5 wt % in the composition, B may be at 5 wt % in the composition, or the combination of A and B may be present at a total of 5 wt % (A+B=5 wt %). Alternatively, where both A and B are present, A may be at 5 wt % and B may be at 5 wt %, totaling 10 wt %.

When referring to the amount present for one or more ingredients, the terms “or ranges including and/or spanning the aforementioned values” (and variations thereof) is meant to include any range that includes or spans the aforementioned values. For example, when the wt % of an ingredient is expressed as “1%, 5%, 10%, 20%, or ranges including and/or spanning the aforementioned values,” this includes wt % ranges for the ingredient spanning from 1% to 20%, 1% to 10%, 1% to 5%, 5% to 20%, 5% to 10%, and 10% to 20%.

As used herein, the term “phytocannabinoid” refers to a group of cannabinoids that occur naturally in the cannabis plant, including but not limited to, THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran).

As used herein, the term “cannabinoid” refers to the chemical substance, regardless of structure or origin, that joins the cannabinoid receptors of the body and brain and that have similar effects to those produced by the cannabis plant. As used herein, the term “cannabinoid” includes but is not limited to Cannabichromenes (e.g., cannabichromene (CBC), cannabichromenic acid (CBCA), cannabichromevarin (CBCV), cannabichromevarinic acid (CBCVA)), Cannabicyclols (e.g., cannabicyclol (CBL), cannabicyclolic acid (CBLA), cannabicyclovarin (CBLV), etc.), Cannabidiols (e.g., cannabidiol (CBD), cannabidiol monomethylether (CBDM), cannabidiolic acid (CBDA), Cannabidiol-C4(CBD-C4), cannabidiorcol (CBD-C1), cannabidivarin (CBDV), cannabidivarinic acid (CBDVA), etc.), Cannabielsoins (e.g., Cannabielsoic Acid (CBEA), Cannabielsoic acid A (CBEA-A), cannabielsoic acid B (CBEA-B), cannabielsoin (CBE), cannabielsoin acid A (CBEA-A), etc.), Cannabigerols (e.g., cannabigerol (CBG), cannabigerol monomethylether (CBGM), cannabigerolic acid (CBGA), cannabigerolic acid monomethylether (CB GAM), cannabigerovarin (CBGV), cannabigerovarinic acid (CBGVA), etc.), Cannabinols and cannabinodiols (e.g., cannabinodiol (CBND), cannabinodivarin (CBVD), cannabinol (CBN) cannabinol methylether (CBNM), cannabinol-C2 (CBN-C2), cannabinol-C4 (CBN-C4), cannabinolic acid (CBNA), cannabiorcool (CBN-C1), cannabivarin (CBV), etc.), Cannabitriols (e.g., 10-ethoxy-9-hydroxy-delta-6a-tetrahydrocannabinol, 8,9-dihydroxy-delta-61-tetrahydrocannabinol, 8,9-Dihydroxy-Δ^{6a(10a)}-tetrahydrocannabinol (8,9-Di-OH-CBT-05), cannabitriol (CBT), cannabitriolvarin (CB TV), Ethoxy-cannabitriolvarin (CBTVE), etc.), tetrahydrocannabinols (e.g., tetrahydrocannabinol (THC), tetrahydrocannabinol-C4 (THC-C4), delta-9-tetrahydrocannabinol (A9-THC), delta-9-tetrahydrocannabinol-C4 (A9-THC-C4), delta-9-tetrahydrocannabinolic acid A (THCA-A), Tetrahydrocannabinolic Acid (THCA), delta-9-tetrahydrocannabinolic acid B (THCA-B) delta-9-tetrahydrocannabinolic acid-C4 (THCA-C4), Tetrahydrocannbinol C4 (THC-C4), delta-9-tetrahydrocannabiorcol (THC-C1), delta-9-tetrahydrocannabiorcolic acid (THCA-C1), delta-9-tetrahydrocannabivarin (THCV), delta-9-tetrahydrocannabivarinic acid (THCVA), delta-9-cis-tetrahydrocannabinol (cis-THC), trihydroxy-delta-9-tetrahydrocannabinol (triOH-THC), etc.), and/or other cannabinoids (e.g., 10-Oxo-delta-6a-tetrahydrocannabinol (OTHC), cannabichromanon (CBCF), cannbifuran (CBF), cannabiglendol, cannabiripsol (CBR), cannbicitran, dehydrocannabifuran (DCBF), 3,4,5,6-tetrahydro-7-hydroxy-alpha-alpha-2-trimethyl-9-n-propyl-2,6-methano-2H-1-benzoxocin-5-methanol (OH-iso-HHCV), Δ⁷-cis-iso-tetrahydrocannabivarin, Δ⁸-tetrahydrocannabinolic acid (A8-THCA), tetrahydrocannabiorcolic acid (THCA-C4), Cannabivarinodiolic (CBNDVA), Cannabivarinodiol (CBNDV), Δ⁸-tetrahydrocannabinol (Δ⁸-THC), Cannabivarinselsoin (CBEV), Cannabivarinselsoinic Acid (CBEVA), Cannabielvarinsoin (CBLV), Cannabielvarinsoinic Acid (CBLVA), Cannabivarinic Acid (CBNVA), Cannabiorcol (CBN-C1), Cannabinodiolic Acid (CBNDA), and/or 3,4,5,6-tetrahydro-7-hydroxy-alpha-alpha-2-trimethyl-9-n-propyl-2,6-methano-2H-1-benzoxocin-5-methanol (OH-iso-HHCV). Cannabinoids can also include cannabinoids derived from sources other than hemp or marijuana, such as oranges. Cannabinoids can also include synthetic (e.g., not naturally occurring, such as analogs, or naturally occurring but synthesized in a lab) chemicals.

As used herein, the term “phospholipid” refers to a lipid having two hydrophobic fatty acid tails and a hydrophilic head comprising of a phosphate group.

As used herein, the term “medium chain triglyceride” refers to tri-substituted triglycerides with fatty acids having aliphatic tails of 6 to 12 carbon atoms (6, 7, 8, 9, 10, 11, 12) and mixtures thereof.

As used herein, the term “long chain triglyceride” refers to tri-substituted triglycerides with fatty acids having an aliphatic tail of greater than 13 carbon atoms (13, 14, 15, 16, 17, 18, 19, 20, or more) and mixtures thereof.

As used herein, the term “sterol” refers to a subgroup of steroids with a hydroxyl group at the 3-position of the A-ring.

As used herein, the term “Cmax” is given its plain and ordinary meaning and refers to the maximum (or peak) plasma concentration of an agent after it is administered.

As used herein, the term “Tmax” is given its plain and ordinary meaning and refers to the length of time required for an agent to reach maximum plasma concentration after the agent is administered.

As used herein, the term “AUC” is given its plain and ordinary meaning and refers to the calculated area under the curve, referring to a plasma concentration-time curve (e.g., the definite integral in a plot of drug concentration in blood plasma vs. time.).

As used herein, “polydispersity” or “PDI” is used to describe the degree of non-uniformity of a size distribution of particles. Also known as the heterogeneity index, PDI is a number calculated from a two-parameter fit to the correlation data (the cumulants analysis). This index is dimensionless and scaled such that values smaller than 0.05 are mainly seen with highly monodisperse standards.

As used herein, an “amino acid” includes amino acids with natural amino acid side chains or non-natural amino acid side chains. As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine. As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

### Introduction

CBD is a prominent phytocannabinoid constituent of Cannabis sativa (Cannabis) that lacks the psychoactive effects of Δ9-tetrahydrocannabinol. CBD was first isolated from Cannabis in 1940 and structurally characterized in 1963. CBD may have broad therapeutic properties across a range of disorders including anxiety, depression, inflammation, pain, and seizure disorders either when administered alone or with THC. Evidence of CBD's therapeutic properties is largely limited to preclinical studies. However, in June 2018 the FDA granted approval of Epidiolex, a CBD isolated from marijuana for the treatment of pediatric seizure disorders, proving CBD's benefit in a controlled clinical trial setting.

With CBD's rise in popularity, consumers are exploring its purported benefits in high numbers. Retail sales of hemp-derived CBD products in the United States reached more than $350 million in 2018 and are expected to reach over $1.3 billion within the next 5 years. As the CBD market flourishes, many CBD manufacturers have come under government scrutiny for making unsubstantiated claims of the health benefits of their compositions or reporting inaccurate lab test results. Being that CBD (and other cannabinoids and phytocannabinoids) is currently available as an unregulated supplement, the quality and safety of consumer CBD products lacks sufficient characterization and laboratory testing. In 2017 survey, 69% of consumer CBD products (n=84) in the categories of oils, tinctures, and vaporizing liquids were found to be reported inaccurately (more than ±10% than label claim), underscoring the need for industrial producers and regulatory agencies to take steps to ensure CBD products are sufficiently characterized and tested. Additionally, the variations in purities of ingredients used to prepare these products give them disperse efficacies and impurity profiles.

For instance, current available compositions for the delivery of CBD to a subject employ CBD as an oil extract. These CBD oils are disadvantageous for a variety of reasons. First, CBD oils exist in an oil state because they include impurities (e.g., agents that prevent the solidification of CBD). Second, those impurities vary from batch-to-batch, making the quality of CBD variable. Additionally, because CBD supplementation thus far has been largely unregulated, variations in CBD concentration and its impurity profile go largely unchecked. Some CBD oils may include THC or other agents. THC is the psychoactive agent in Cannabis sativa. It would be advantageous to use CBD and other cannabinoids in highly pure forms to avoid such impurities (such as THC), because those impurities could result in patients avoiding CBD and cannabinoid therapies altogether.

Exacerbating the issue, current CBD formulations use CBD oils to sufficiently disperse the compound and to form particles. Further compounding the issues with CBD impurities, the ingredients used to form lipophilic particles comprising CBD also have a wide variety of impurities and variations batch-to-batch. Moreover, the lipophilic compositions using CBD oil often rely, at least in part, on a distribution and/or variety of lipophilic impurities in each of the liposomal ingredients to aid in dispersing CBD. Because current delivery systems must use CBD oils and lipophilic ingredients with a distribution of compounds in order to sufficiently solubilize the compound and because CBD oils and the lipophilic ingredients used to solubilize them comprise impurities, delivery and stability of these mixtures is unpredictable. These impurities may also lead to side effects. Thus, new delivery systems that are able to utilize highly pure CBD forms and pure and/or uniform ingredients are needed. Several of these issues are pertinent to other therapeutic agents, including other cannabinoids and non-cannabinoid therapeutics (both hydrophobic and hydrophilic), and combinations thereof.

Causing additional issues, CBD oils and certain other cannabinoid and non-cannabinoid therapeutics have low bioavailability (due to poor absorption and due to their variable purity profile). Highly pure isolate forms of CBD and certain other cannabinoid and non-cannabinoid therapeutics perform even worse from a formulation and pharmacokinetic standpoint and have poorer bioavailability. For instance, CBD isolate forms have low oral bioavailability due to low solubility in aqueous systems (e.g., and in the gut, etc.). Highly purified CBD exists as a solid isolate (e.g., a powder or crystalline form). These highly purified powders heretofore have not been formulated for oral delivery due to their prohibitively high aqueous insolubility (e.g., hydrophobicity). Indeed, it is believed that, prior to the lipid-based particle compositions and methods disclosed in the present disclosure, solid CBD isolate powder had not been provided in any delivery system to facilitate solubility and absorption. This is apparent from the impurity profile for commercial CBD products. As noted above, available CBD delivery systems make use of CBD oil. These systems have been shown to be ineffective for high purity CBD (such as a CBD crystalline composition or powder). The ability to facilitate the solubility, absorption, and stability of CBD highlights the beneficial properties of the lipid-based particle compositions provided herein.

Some embodiments disclosed herein solve these or other problems by providing a lipid-based particle composition that can delivery highly pure CBD, other cannabinoids, other therapeutic agents, and/or combinations of any of the foregoing in a solubilizing particle delivery system (e.g., lipid nanoparticle, a liposomal system, oil-in-water emulsions, dry liposome particles, etc.). For example, in some embodiments, disclosed herein are lipid-based particle compositions comprising therapeutic agents or combinations of therapeutic agents. In some embodiments, the disclosed lipid-based particle compositions achieve one or more of the following (or other benefits): they include less impurities, they have less variations batch-to-batch (e.g., stability, degradation profiles, efficacy), they have more delivery predictability, they less side effects when treating a patient, they have higher bioavailability, they have faster onset of activity, they have better efficacy, etc. Disclosed herein are therapeutic lipid-based particle products comprising CBD, other cannabinoids, other therapeutic agents, and/or combinations of any of the foregoing prepared using the thoroughness and diligence of pharmaceutical drug development to consumer products. In some embodiments, a nano-lipid delivery system is utilized to impart apparent aqueous solubility and deliverability to an otherwise practically water insoluble molecule (e.g., CBD or other similar and/or hydrophobic phytocannabinoids and therapeutic molecules). In some embodiments, as disclosed herein, attributes of some embodiments disclosed herein have been determined to be high quality and reproducible. Such reproducibility and low variations may allow the products to generate a reproducible certificate of analysis for different batches.

In some embodiments, the systems disclosed herein (e.g., lipid-based particle compositions and/or formulations comprising them) increase the bioavailability of therapeutic agents (e.g., CBD, cannabinoids, other therapeutic agents, and/or combinations of any of the foregoing), decrease the time for absorption of those therapeutic agents, increase the stability of the therapeutic agents or the particles comprising the therapeutic agents, increase the consistency of delivery (e.g., by limiting batch-to-batch variation), and/or increase the efficacy of the therapeutic agents (higher dosing and/or faster onset of activity).

As disclosed elsewhere herein, in some embodiments, the carriers (lipid-based particle compositions) disclosed herein are able to deliver therapeutic agents that are highly pure. In some embodiments, the therapeutic agents (e.g., cannabinoids, such as CBD, non-cannabinoids, and combinations thereof) have a purity of greater than or equal to about: 90%, 95%, 98%, 99%, 99.5%, 99.9%, 99.99%, or ranges including and/or spanning the aforementioned values. In some embodiments, for example, the lipid-based particle compositions disclosed herein make use of CBD or other therapeutic agents that is of sufficient purity that the it exists as a solid (e.g., a powder, a crystalline compound, etc.). In some embodiments, the solid therapeutic agents are solid due to high levels of purity and lacking other agents that would cause it to form an oil when impure. For example, in some embodiments, the CBD powder (or other therapeutic agents) lack solidifying agents, such as, maltodextrin or other additive agents that cause the solidification of CBD (or other therapeutic agents).

As disclosed elsewhere herein, some embodiments relate to delivery systems (e.g., lipid-based particle compositions and/or formulations comprising the same) that improve the absorption of the highly insoluble forms of a therapeutic agent (e.g., CBD) or combination of agents. In some embodiments, the therapeutic agent (e.g., CBD, non-cannabinoid therapeutics, etc.) used to prepare the lipid-based particle compositions disclosed herein (e.g., the starting material) has an aqueous solubility of less than or equal to about: 0.05 mg/ml, 0.01 mg/ml, 0.012 mg/ml, 0.001 mg/ml, or ranges including and/or spanning the aforementioned values. In some embodiments, where a combination of therapeutic agents is used to prepare the lipid-based particle compositions disclosed herein one or more or all of the therapeutic agents in the composition has an aqueous solubility of less than or equal to about: 0.05 mg/ml, 0.01 mg/ml, 0.012 mg/ml, 0.001 mg/ml, or ranges including and/or spanning the aforementioned values. In some embodiments, the aqueous solubility of the therapeutic agent or agents (and/or the amount of therapeutic agent or agents that can be provided in an aqueous solution) can be improved to equal to or greater than about: 1 mg/ml, 5 mg/ml, 20 mg/ml, 30 mg/ml, 50 mg/ml, 100 mg/ml, or ranges including and/or spanning the aforementioned values.

### Therapeutic Agents

In some embodiments, at least one therapeutic agent in the lipid-based particle composition (and/or combination of therapeutic agents provided in the lipid-based particle composition) is hydrophobic. In some embodiments, at least one hydrophobic therapeutic agent used to prepare a lipid-based particle composition as disclosed herein (e.g., cannabinoid, a phytocannabinoid, vitamin, or other therapeutic agent, etc.) has an aqueous solubility of less than or equal to about: 0.05 mg/ml, 0.01 mg/ml, 0.012 mg/ml, 0.001 mg/ml, or ranges including and/or spanning the aforementioned values. In some embodiments, the solubility of the at least one hydrophobic therapeutic agent (and/or the amount of the therapeutic that can be provided in an aqueous solution) used to prepare the compositions disclosed herein (e.g., a cannabinoid, etc.) can be improved to equal to or greater than about: 1 mg/ml, 5 mg/ml, 20 mg/ml, 30 mg/ml, 50 mg/ml, 100 mg/ml, or ranges including and/or spanning the aforementioned values. In some embodiments, the solubility of the at least one hydrophobic therapeutic agent (including CBD) can be improved by at least about: 50%, 100%, 150%, 200%, 500%, 1000%, 10,000%, or ranges including and or spanning the aforementioned values. In some embodiments, the solubility is measured as an amount that can be suspended for longer than 30 days and or that can be dissolved in an aqueous solution at a concentration of at least 20 mg/ml.

In several embodiments, the therapeutic agent(s) is or may be synthetic. In several embodiments, the therapeutic agent(s) is or may be non-synthetic. In several embodiments, the therapeutic agent(s) is or may be semi-synthetic (e.g., prepared through fermentation, etc.). In several embodiments, the therapeutic agent(s) is or may be a combination of synthetic and non-synthetic therapeutic agents.

In several embodiments, the therapeutic agent is a single compound (or is substantially a single compound). In several embodiments, the therapeutic agent comprises mixture of different compounds (e.g., comprises a full spectrum of compounds) provided as an extract from a therapeutic agent source (e.g., cannabinoids other therapeutics extracted from a source, such as a plant). In several embodiments, the therapeutic agent is a distillate from a therapeutic agent source. For example, in several embodiments, the therapeutic agent is a hemp distillate. In several embodiments, the therapeutic agent is a cannabinoid distillate from a cannabinoid source.

In some embodiments, as disclosed elsewhere herein, a lipid-based particle composition (e.g., a liposomal, solid lipid particles, oil-in-water emulsions, nanoparticle, etc.) is provided to aid in the delivery of therapeutic agents. In some embodiments, when formulated, the dry weight % of one or more therapeutic agents (e.g., CBD) present in the compositions is equal to or at least about: 0.5%, 1%, 5%, 7.5%, 10%, 15%, 20%, 25%, 50%, 60%, 70%, or ranges including and/or spanning the aforementioned values. In some embodiments, the therapeutic agents are provided in an aqueous composition. In some embodiments, the wet weight % of one or more therapeutic agents (e.g., CBD) present in the composition (with water included) is equal to or at least about: 0.1%, 0.5%, 0.75%, 1%, 1.5%, 2%, 3%, 4%, 5%, 7.5%, 10%, 15%, 20%, 25%, or ranges including and/or spanning the aforementioned values. In some embodiments, the one or more therapeutic agents (e.g., CBD) may be provided in the wet composition at a concentration of greater than or equal to about: 1 mg/ml, 5 mg/ml, 20 mg/ml, 30 mg/ml, 50 mg/ml, 100 mg/ml, 150 mg/ml, 200 mg/ml, or ranges including and/or spanning the aforementioned values.

In some embodiments, as disclosed elsewhere herein, the one or more therapeutic agents (e.g., cannabinoids, such as CBD, non-cannabinoids, and combinations thereof) used in the lipid-based particle compositions as disclosed herein has high purity as indicated by its existing in a solid form (e.g., powder) prior to processing (e.g., formulation into a composition as disclosed herein). In some embodiments, using the combinations disclosed herein, a composition comprising one or more therapeutic agents (e.g., cannabinoids, such as CBD, non-cannabinoids, and combinations thereof) in water is provided. In some embodiments, as disclosed elsewhere herein, the delivery system may be lipid-based and forms an oil-in-water emulsion (e.g., a nanoemulsion), a liposome, and/or solid lipid particle (e.g., nanoparticle). In some embodiments, the lipid-based delivery system provides particles in the nano-measurement range (as disclosed elsewhere herein). In some embodiments, a solid lipid nanoparticle is spherical or substantially spherical nanoparticle. In some embodiments, a solid lipid nanoparticle possesses a solid lipid core matrix that can solubilize lipophilic molecules. In some embodiments, the lipid core is stabilized by surfactants and/or emulsifiers as disclosed elsewhere herein, while in other embodiments, surfactants are absent. In some embodiments, the size of the particle is measured as a mean diameter. In some embodiments, the size of the particle is measured by dynamic light scattering. In some embodiments, the size of the particle is measured using a zeta-sizer. In some embodiments, the size of the particle can be measured using Scanning Electron Microscopsy (SEM). In some embodiments, the size of the particle is measured using a cyrogenic SEM (cryo-SEM). Where the size of a nanoparticle is disclosed elsewhere herein, any one or more of these instruments or methods may be used to measure such sizes.

In some embodiments, as disclosed elsewhere herein, the lipid-based particle and/or nanoparticle composition (e.g., a liposomal composition as disclosed herein, a solid lipid particle composition as disclosed herein, an oil-in-water emulsion composition as disclosed herein, etc.), or simply the composition for brevity, comprises a therapeutic agent or combination of therapeutics (e.g., one or more cannabinoids, phytocannabinoids, non-cannabinoid therapeutics) and one or more of a phospholipid, a lipid other than a phospholipid (e.g., a lipid that is not a phospholipid), and a sterol. In some embodiments, as disclosed elsewhere herein, the composition comprises therapeutic agent or combination of therapeutics (e.g., one or more cannabinoids, phytocannabinoids, non-cannabinoid therapeutics), a phospholipid, a lipid other than a phospholipid (e.g., a lipid that is not a phospholipid), and a sterol. In some embodiments, the composition is aqueous (e.g., contains water) while in other embodiments, the composition is dry (lacks water or substantially lacks water). In some embodiments, the composition has been dried (e.g., has been subjected to a process to remove most or substantially all water). In some embodiments, the composition comprises nanoparticles in water (e.g., as a solution, suspension, or emulsion). In other embodiments, the composition is provided as a powder (e.g., that can be constituted or reconstituted in water). In some embodiments, as disclosed elsewhere herein, the water content (in wt %) of the composition is less than or equal to about: 10%, 5%, 2.5%, 1%, 0.5%, 0.1%, or ranges including and/or spanning the aforementioned values.

As disclosed elsewhere herein, in some embodiments, the lipid-based particle composition may include one or more therapeutic agents (e.g., a single therapeutic agent or a combination of therapeutic agents). In some embodiments, the lipid-based particle composition may include a single therapeutic agent or a plurality of therapeutic agents (e.g., 1, 2, 3, 4, or more). For example, the lipid-based particle composition may comprise a cannabinoid and a non-cannabinoid therapeutic agent (e.g., a cannabinoid and a terpene); the lipid-based particle may comprise two cannabinoids and a non-cannabinoid therapeutic agent; the lipid-based particle may comprise two non-cannabinoid therapeutic agents; the lipid-based particle may comprise a non-cannabinoid, hydrophilic therapeutic agent and a hydrophobic active agent (e.g., a cannabinoid or non-cannabinoid), etc.

In some embodiments, the therapeutic agents, collectively or individually, are present in the aqueous lipid-based particle composition at a concentration of less than or equal to about: 200 mg/ml, 150 mg/mL, 100 mg/ml, 75 mg/ml, 50 mg/ml, 25 mg/ml, 20 mg/ml, 10 mg/ml, 5 mg/ml, 2.5 mg/ml or ranges including and/or spanning the aforementioned values. In some embodiments, the one or more therapeutic agents, collectively or individually, are present in the aqueous composition at a concentration of greater than or equal to about: 200 mg/ml, 150 mg/mL, 100 mg/ml, 75 mg/ml, 50 mg/ml, 25 mg/ml, 20 mg/ml, 10 mg/ml, 5 mg/ml, or ranges including and/or spanning the aforementioned values. In some embodiments, the one or more therapeutic agents, collectively or individually, are present in the composition at a dry wt % of equal to or at least about: 0.5%, 1%, 5%, 7.5%, 10%, 15%, 20%, 25%, 50%, 60%, 70%, or ranges including and/or spanning the aforementioned values. In some embodiments, the one or more therapeutic agents, collectively or individually, are present in the composition at a wet wt % of equal to or at least about: 0.1%, 0.25%, 0.5%, 1%, 2%, 3%, 4%, 5%, 7.5%, 10%, 15%, 20%, 25%, or ranges including and/or spanning the aforementioned values. In some embodiments, as disclosed elsewhere herein, the composition is aqueous, while in others it has been dried into a powder (that is free of or substantially free of water). In some embodiments, where the composition has been dried, it comprises a water content of less than or equal to 20%, 15%, 10%, 7.5%, 5%, 2.5%, 1%, or ranges including and/or spanning the aforementioned values.

In some embodiments, as shown in the Examples, the therapeutic agent may comprise or may be a full spectrum or broad spectrum extracts (e.g., hemp extracts). In some embodiments, the therapeutic agent comprises rosin. In some embodiments, the rosin is extract that is produced after pressing cannabis or hemp flower using a high-pressure press. In some embodiments, the rosin is a full spectrum extract. In some embodiments, as shown in the Examples, the therapeutic agent may comprise full spectrum extract, broad spectrum extract, crude, distillates, oils, and isolates, and combinations thereof.

In some embodiments, as disclosed elsewhere herein, the lipid-based particle composition may include one or more cannabinoids (e.g., a single cannabinoid or a combination of different cannabinoids). In some embodiments, the lipid-based particle composition may include one or more phytocannabinoids (e.g., a single phytocannabinoid or a combination of different phytocannabinoids). In some embodiments, as disclosed elsewhere herein, the lipid-based particle composition comprises CBD and at least one other cannabinoid and/or therapeutic agent.

In some embodiments, the lipid-based particle composition comprises a cannabichromene, a cannabicyclol, a cannabidiol, a cannabielsoin, a cannabigerol, a cannabinol, a cannabinodiol, a cannabitriol, a delta-9-tetrahydrocannabinol, another cannabinoid, a synthetic cannabinoid, and/or combinations of any of the foregoing. In some embodiments, the lipid-based particle composition comprises two or more cannabichromenes, cannabicyclols, cannabidiols, cannabielsoins, cannabigerols, cannabinols, cannabinodiols, cannabitriols, delta-9-tetrahydrocannabinols, other cannabinoids, synthetic cannabinoids, and/or combinations of any of the foregoing. In some embodiments, the lipid-based particle composition comprises CBC, CBCA, CBCV, CBCVA, CBL, CBLA, CBLV, CBD, CBDM, CBDA, CBD-C1, CBDV, CBDVA, CBEA-B, CBE, CBEA-A, CBG, CBGM, CBGA, CBGAM, CBGV, CBGVA, CBND, CBVD, CBN, CBNM, CBN-C2, CBN-C4, CBNA, CBN-C1, CBV, 10-ethoxy-9-hydroxy-delta-6a-tetrahydrocannabinol, 8,9-dihydroxy-delta-61-tetrahydrocannabinol, CBT, CBTV, THC, THC-C4, THCA-A, THCA-B, THCA-C4, THC-C1, THCA-C1, THCV, THCVA, OTHC, CBCF, CBF, cannabiglendol, CBR, cannbicitran, DCBF, cis-THC, triOH-THC, OH-iso-HHCV, synthetic cannabinoids, and/or combinations of any of the foregoing. For example, in some embodiments, the lipid-based particle composition comprises CBN, CBD, and CBG. In some embodiments, the cannabinoid or cannabinoids, collectively or individually, are present in the aqueous lipid-based particle composition at a concentration of less than or equal to about: 200 mg/ml, 150 mg/ml, 100 mg/ml, 75 mg/ml, 50 mg/ml, 25 mg/ml, 20 mg/ml, 10 mg/ml, 5 mg/ml, 2.5 mg/ml or ranges including and/or spanning the aforementioned values. In some embodiments, the cannabinoid or cannabinoids, collectively or individually, are present in the aqueous composition at a concentration of greater than or equal to about: 100 mg/ml, 75 mg/ml, 50 mg/ml, 25 mg/ml, 20 mg/ml, 10 mg/ml, 5 mg/ml, or ranges including and/or spanning the aforementioned values. In some embodiments, the cannabinoid or cannabinoids, collectively or individually, are present in the composition at a dry wt % of equal to or at least about: 0.5%, 1%, 5%, 7.5%, 10%, 15%, 20%, 25%, 50%, 60%, 70%, or ranges including and/or spanning the aforementioned values. In some embodiments, the cannabinoid or cannabinoids, collectively or individually, are present in the composition at a wet wt % of equal to or at least about: 0.1%, 0.25%, 0.5%, 1%, 2%, 3%, 4%, 5%, 7.5%, 10%, 15%, 20%, 25%, or ranges including and/or spanning the aforementioned values.

In some embodiments, for non-THC compositions (e.g., including cannabinoids without delta-9-tetrahydrocannabinols), the total potential THC does not to exceed 0.3 weight % of the phytocannabinoid, where the total potential THC is defined as THCa×0.877+9-THC+8-THC. In some embodiments, for non-THC compositions (e.g., including cannabinoids without THC), the total potential THC does not to exceed 0.3 weight % of the phytocannabinoid, where the total potential THC is defined as THCa+9-THC.

In some embodiments, as disclosed elsewhere herein, instead of a cannabinoids (e.g., phytocannabinoid(s)) or in addition to cannabinoids (e.g., CBD), the lipid-based particle composition may comprise a non-cannabinoid therapeutic agent or non-cannabinoid active agents (e.g., agents that are not a cannabinoid). In some embodiments, the non-cannabinoid therapeutic agent is one or more of a vitamin, a nutrient, a plant extract, a nutraceutical, a pharmaceutical, or another beneficial agent. In some embodiments, the non-cannabinoid therapeutic agent is hydrophilic. In some embodiments, the non-cannabinoid therapeutic agent is hydrophobic. In some embodiments, the non-cannabinoid therapeutic agent is amphiphilic.

In some embodiments, the non-cannabinoid therapeutic agent is selected from the group consisting of Noopept (N-phenylacetyl-L-prolyglygice ethyl ester), melatonin, glutathione, gamma-glutamylcysteine (GGC), gamma-aminobutyric acid (GABA), valerian root, magnesium, theanine, 5-HTP, tyrosine, taurine, zinc, alpha fenchone, alpha terpinene, alpha terpineol, beta caryophyllene, alpha pinene, beta pinene, bisabolene, bisabolol, borneol, eucalyptol, gamma terpinene, guaiacol, humulene, linalool, myrcene, para cymene, phytol, terpinolene, limonene, others, and/or combinations thereof. In some embodiments, as disclosed elsewhere herein, these non-cannabinoid therapeutic agents may be provided in combination with cannabinoids at the concentrations disclosed herein. In some embodiments, when a hydrophilic composition is used, it is mixed with the aqueous soluble ingredients before mixing with the lipid ingredients.

In some embodiments, as disclosed elsewhere herein, the one or more non-cannabinoid therapeutic agent(s), collectively or individually, are present in the aqueous composition at a concentration of greater than or equal to about: 200 mg/ml, 150 mg/ml, 100 mg/ml, 75 mg/ml, 50 mg/ml, 25 mg/ml, 20 mg/ml, 10 mg/ml, 5 mg/ml, or ranges including and/or spanning the aforementioned values. In some embodiments, the one or more non-cannabinoid therapeutic agent(s) (collectively or individually) are present in the composition at a dry wt % of equal to or at least about: 0.5%, 1%, 5%, 7.5%, 10%, 15%, 20%, 25%, 50%, 60%, 70%, or ranges including and/or spanning the aforementioned values. In some embodiments, the one or more non-cannabinoid therapeutic agent(s) (collectively or individually) are present in the composition at a wet wt % of equal to or at least about: 0.1%, 0.25%, 0.5%, 1%, 2%, 3%, 4%, 5%, 7.5%, 10%, 15%, 20%, 25%, or ranges including and/or spanning the aforementioned values.

As disclosed elsewhere herein, in some embodiments, combination products are provided. In some embodiments, combination products may include one or more cannabinoids, one or more non-cannabinoid therapeutic agents, or mixtures of any the foregoing. Some exemplary compositions are provided below.

In some embodiments, lipid-based particle compositions as disclosed herein may be configured for use as a sleep aid formulation, for use in methods of inducing sleep, and/or for methods of treating sleep disorders. In some embodiments, the lipid-based particle composition for use as a sleep aid comprises one or more of CBN, CBD, and/or CBG. In some embodiments, an aqueous sleep aid lipid-based particle composition comprises CBN in an amount equal to or less than about: 100 mg/ml, 75 mg/ml, 50 mg/ml, 20 mg/ml, 10 mg/ml, 5 mg/ml, or ranges including and/or spanning the aforementioned values. In some embodiments, an aqueous sleep aid lipid-based particle composition comprises CBD in an amount equal to or less than about: 100 mg/ml, 75 mg/ml, 50 mg/ml, 20 mg/ml, 10 mg/ml, 5 mg/ml, 2 mg/ml, 1 mg/ml or ranges including and/or spanning the aforementioned values. In some embodiments, an aqueous sleep aid lipid-based particle composition comprises CBG in an amount equal to or less than about: 100 mg/ml, 75 mg/ml, 50 mg/ml, 20 mg/ml, 10 mg/ml, 5 mg/ml, 2 mg/ml, 1 mg/ml or ranges including and/or spanning the aforementioned values. In some embodiments, an aqueous sleep aid lipid-based particle composition comprises CBN in an amount equal to or less than about 20 mg/ml, CBD in an amount equal to or less than about 2 mg/ml, and CBG in an amount equal to or less than about 1 mg/ml. In some embodiments, an aqueous sleep aid lipid-based particle composition comprises CBN in an amount equal to or less than about 10 mg/ml, CBD in an amount equal to or less than about 2 mg/ml, and CBG in an amount equal to or less than about 1 mg/ml.

In some embodiments, the sleep aid formulation comprises one or more of valerian root, magnesium, GABA, galantamine, melatonin, theanine, 5-HTP, tyrosine, taurine, zinc (e.g., non-cannabinoid therapeutic agents in the amounts as disclosed elsewhere herein). These ingredients may be provided in addition to CBN, CBD, and/or CBG or as alternatives to any one of these cannabinoids. In some embodiments, the sleep aid formulation comprises THC (in the amounts as disclosed elsewhere herein). Any one or more of valerian root, magnesium, GABA, galantamine, melatonin, theanine, 5-HTP, tyrosine, zinc, and/or taurine may be provided in addition to one or more cannabinoid in some embodiments of sleep aid formulations. In some embodiments, the lipid-based particle composition comprising one or more of valerian root, magnesium, GABA, galantamine, melatonin, theanine, 5-HTP, tyrosine, taurine, zinc, and/or one or more cannabinoids is configured for use as a use in a method of treating insomnia or sleeplessness.

In some embodiments, the sleep aid formulation comprises one or more of limonene, myrcene, alpha-terpinene, gamma-terpinene, linalool, and/or terpinolene maybe included (e.g., non-cannabinoid therapeutic agents in the amounts as disclosed elsewhere herein). In some embodiments, one or more cannabinoids (e.g., CBN, CBD, CBG, etc.) may be added to any one or more of limonene, myrcene, alpha-terpinene, gamma-terpinene, linalool, and/or terpinolene to provide a sleep aid formulation. In some embodiments, the lipid-based particle composition comprising one or more of limonene, myrcene, alpha-terpinene, gamma-terpinene, linalool, terpinolene, and/or one or more cannabinoids is configured for use as a use in a method of treating insomnia or sleeplessness.

In some embodiments, the lipid-based particle composition comprises one or more of alpha-fenchome, guaiacel, para-cymene, and/or beta-camophyliene (e.g., non-cannabinoid therapeutic agents in the amounts as disclosed elsewhere herein). In some embodiments, the lipid-based particle composition comprising one or more of alpha-fenchome, guaiacel, para-cymene, and/or beta-camophyliene is configured for use as a pain relief formulation and/or is used in methods of treating pain. In some embodiments, the lipid-based particle composition comprising alpha-fenchome, guaiacel, para-cymene, and/or beta-camophyliene is configured for oral or topical use in pain relief (e.g., to treat pain). In some embodiments, alpha-fenchome, guaiacel, para-cymene, and/or beta-camophyliene may be provided with CBD or other cannabinoids (e.g., in the amounts disclosed elsewhere herein) or without cannabinoids.

In some embodiments, the lipid-based particle composition comprises one or more of alpha-terpineol and/or phytol (e.g., non-cannabinoid therapeutic agents in the amounts as disclosed elsewhere herein). In some embodiments, the lipid-based particle composition comprising alpha-terpineol and/or phytol is configured for use as an anti-anxiety formulation (e.g., to treat anxiety) and/or is used in methods of treating anxiety. In some embodiments, alpha-terpineol and/or phytol may be provided with CBD or other cannabinoids (e.g., in the amounts disclosed elsewhere herein) or without cannabinoids.

In some embodiments, the lipid-based particle composition comprises alpha-fenchome, alpha-terpineol, and/or bisabolol (in the amounts as disclosed elsewhere herein). In some embodiments, the lipid-based particle composition comprising alpha-fenchome, alpha-terpineol, and/or bisabolol is configured for use as an anti-inflammatory formulation and/or is used in a method of reducing and/or treating inflammation. In some embodiments, alpha-fenchome, alpha-terpineol, and/or bisabolol may be provided with CBD or other cannabinoids (e.g., in the amounts disclosed elsewhere herein) or without cannabinoids.

In some embodiments, the lipid-based particle composition comprises one or more of alpha-terpineol and/or canephene. In some embodiments, the lipid-based particle composition comprising alpha-terpineol and/or canephene is configured for use as an anti-oxidant and/or is used in a method of lowering oxidative damage in the body of a subject. In some embodiments, alpha-terpineol and/or canephene may be provided with CBD or other cannabinoids (e.g., in the amounts disclosed elsewhere herein) or without cannabinoids.

In some embodiments, as disclosed elsewhere herein, the CBD or other therapeutic agent is a purified form. As disclosed elsewhere herein, in some embodiments, the CBD or other therapeutic used to prepare the lipid-based particle composition is a solid and not an oil (e.g., is a CBD of sufficiently high purity that it exists as a solid). In some embodiments, the CBD (or other non-THC cannabinoid) is an isolate having a THC (including all THC isomers and stereoisomers) content (in weight %) of less than or equal to about: 0.01%, 0.1%, 0.3%, 0.5%, 1.0%, 3.0%, 4.0%, 5.0%, or ranges including and/or spanning the aforementioned values. In some embodiments, the CBD (or other non-THC cannabinoid) has a total potential THC content (in weight %) of less than or equal to about: 0.01%, 0.1%, 0.3%, 0.5%, 1.0%, 3.0%, 4.0%, 5.0%, or ranges including and/or spanning the aforementioned values. In some embodiments, the CBD (or other non-THC cannabinoid) is substantially THC free, lacks THC, or lacks a detectable amount of THC. In some embodiments, the CBD (or other non-THC cannabinoid) is isolated from hemp and/or marijuana. In some embodiments, the CBD (or other non-THC cannabinoid) is isolated from hemp and not marijuana. In some embodiments, the CBD (or other non-THC cannabinoid) is isolated from marijuana and not hemp. In some embodiments, the CBD (or other cannabinoid) has a terpene impurity content (in weight percent) of less than or equal to about: 0.01%, 0.1%, 0.3%, 0.5%, 1.0%, 2.0%, 5.0% or ranges including and/or spanning the aforementioned values.

### Phospholipids

As disclosed elsewhere herein, in some embodiments, the lipid-based particle composition comprises one or more phospholipids. In some embodiments, the one or more phospholipids comprises one or more of phosphatidic acid, phosphatidylethanolamine, phosphatidylcholine, phosphatidylserine, phosphatidylinositol, phosphatidylinositol phosphate, phosphatidylinositol bisphosphate, and phosphatidylinositol trisphosphate. In some embodiments, the phospholipid is phosphatidylcholine. In some embodiments, the only phospholipid present is phosphatidylcholine (e.g., the phospholipid lacks phospholipids other than phosphatidylcholine or is substantially free of other phospholipids). In some embodiments, the one or more phospholipid components (e.g., phosphatidylcholine, and/or others), collectively or individually, are present in the aqueous composition at a concentration of less than or equal to about: 400 mg/ml, 300 mg/ml, 200 mg/ml, 150 mg/ml, 100 mg/ml, 75 mg/ml, 50 mg/ml, 25 mg/ml, or ranges including and/or spanning the aforementioned values. For instance, as disclosed elsewhere herein, where two phospholipids are present (e.g., phosphatidylcholine and phosphatidylethanolamine), those phospholipids may be present collectively at a concentration of 50 mg/ml (e.g., 30 g/ml phosphatidylcholine and 20 g/ml phosphatidylethanolamine=50 mg/ml total) or individually at a concentration of 50 mg/ml (e.g., 50 g/ml phosphatidylcholine and 50 g/ml phosphatidylethanolamine). In some embodiments, the one or more phospholipid(s) (collectively or individually) are present in the composition at a dry wt % of equal to or less than about: 5%, 10%, 15%, 20%, 25%, 30%, 40%, 50%, 60%, or ranges including and/or spanning the aforementioned values. In some embodiments, the one or more phospholipid(s) (collectively or individually) are present in the composition at a wet wt % of equal to or less than about: 2.5%, 5%, 7.5%, 10%, 12.5%, 15%, 20%, 25%, 30%, 40%, or ranges including and/or spanning the aforementioned values. In some embodiments, as disclosed elsewhere herein, the composition is aqueous, while in others it has been dried into a powder. In some embodiments, the phosphatidylcholine is synthetic, derived from sunflower, soy, egg, or mixtures thereof. In some embodiments, the one or more phospholipids (and/or lipids) can be hydrogenated or non-hydrogenated.

In some embodiments, where a phospholipid (e.g., phosphatidylcholine) is used, the phospholipid (e.g., phosphatidylcholine) may be of high purity. For example, in some embodiments, the phosphatidylcholine is H100-3 grade (from Lipoid) and includes over 96.3% phosphatidylcholine (hydrogenated) or over 99% phosphatidylcholine (hydrogenated). In some embodiments, the phospholipid (e.g., phosphatidylcholine) has a purity of greater than or equal to about: 92.5%, 95%, 96%, 96.3%, 98%, 99%, 100%, or ranges including and/or spanning the aforementioned values. In some embodiments, the phospholipid (e.g., phosphatidylcholine) has a total % impurity content by weight of less than or equal to about: 8.5%, 5%, 4%, 3.7%, 2%, 1%, 0%, or ranges including and/or spanning the aforementioned values. In some embodiments, the phospholipid (e.g., phosphatidylcholine) comprises less than or equal to about 8.5%, 5%, 4%, 3.7%, 2%, 1%, or 0.1% (or ranges including and/or spanning the aforementioned values) of any one or more of saturated fatty acids, monounsaturated fatty acids, polyunsaturated fatty acids (C 18), arachidonic acid (ARA) (C 20:4), docosahexaenoic acid DHA (C 22:6), phosphatidic acid, phosphatidylethanolamine, and/or lysophosphatidylcholine by weight. In some embodiments, the phosphatidylcholine has less than about 1.1% lysophosphatidylcholine and less than about 2.0% triglycerides by weight.

### Sterols

As disclosed elsewhere herein, in some embodiments, the lipid-based particle composition comprises one or more sterols. In some embodiments, the one or more sterols comprises one or more cholesterols, ergosterols, hopanoids, hydroxysteroids, phytosterols (e.g., vegapure), ecdysteroids, and/or steroids. In some embodiments, the sterol comprises cholesterol. In some embodiments, the sterol is cholesterol. In some embodiments, the only sterol present is cholesterol (e.g., the sterol lacks or substantially lacks sterols other than cholesterol). In some embodiments, the one or more sterol(s) (e.g., cholesterol, and/or other sterols), collectively or individually, are present in the aqueous composition at a concentration of less than or equal to about: 50 mg/ml, 40 mg/ml, 20 mg/ml, 10 mg/ml, 5 mg/ml, or ranges including and/or spanning the aforementioned values. In some embodiments, the one or more sterol(s) are present in the composition at a dry wt % of equal to or less than about: 0.25%, 0.5%, 1%, 5%, 7.5%, 10%, 15%, 20%, 25%, 30%, 40%, or ranges including and/or spanning the aforementioned values. In some embodiments, the one or more sterol(s) (collectively or individually) are present in the composition at a wet wt % of equal to or less than about: 0.1%, 0.25%, 0.5%, 1%, 2%, 3%, 4%, 5%, 7.5%, 10%, 15%, 20%, or ranges including and/or spanning the aforementioned values. In some embodiments, as disclosed elsewhere herein, the composition is aqueous, while in others it has been dried into a powder. In some embodiments, the cholesterol used in the composition comprises cholesterol from one or more of sheep's wool, synthetic cholesterol, or semisynthetic cholesterol from plant origin. In some embodiments, the sterol (or combination of sterols) has a purity of greater than or equal to about: 92.5%, 95%, 96%, 98%, 99%, 99.9%, 100.0%, or ranges including and/or spanning the aforementioned values. In some embodiments, the sterol has a total % impurity content by weight of less than or equal to about: 8.5%, 5%, 4%, 3.7%, 2%, 1%, 0%, or ranges including and/or spanning the aforementioned values. In some embodiments, the sterol is cholesterol. In some embodiments, the sterol is not cholesterol. In some embodiments, the sterol is phytosterol.

### Lipid Components

As disclosed elsewhere herein, in some embodiments, the lipid-based particle composition comprises a lipid (e.g., a lipid that is not a phospholipid). In some embodiments, the lipid (or mixture of lipids) used in the composition is a liquid at room temperature. In some embodiments, the lipid(s) is one in which the therapeutic agent (e.g., CBD) is soluble. In some embodiments, the lipid(s) comprises one or more of a triglyceride(s) and/or one or more oils. In some embodiments, where the lipid is an oil, the oil may be hemp oil and/or marijuana oil. In some embodiments, the lipid (e.g., the triglyceride) comprises one or more medium chain triglycerides (MCTs). In some embodiments, the lipid comprises one or more medium chain triglycerides that can be an ester of glycerol and any one or more medium chain fatty acids. For instance, in some embodiments, the medium chain triglyceride comprises a fatty acid with an aliphatic tail 6-12 carbons in length (e.g., 6, 7, 8, 9, 10, 11, or 12) or combinations of different chain length fatty acids. Thus, in some embodiments, the MCT could comprise a tri-ester of glycerol and one fatty acid having an aliphatic chain length of 8, one fatty acid having an aliphatic chain length of 9, and one fatty acid having an aliphatic chain length of 10. In some embodiments, the MCT could comprise a tri-ester of glycerol and three fatty acid having an aliphatic chain that is the same length (e.g., each having a length of 8). In some embodiments, the medium chain fatty acids of the MCT include one or more of caprioc acid, heptanoic acid, octanoic acid, nonanoic acid, capric acid, undecanoic acid, and/or lauric acid, or any combination thereof. In some embodiments, the lipid comprises tristearin. In some embodiments, the lipid component comprises one or more long chain triglycerides. In some embodiments, the long chain triglyceride comprises a fatty acid having a tail that is greater than 12 carbons in length (e.g., greater than or equal to 13, 14, 15, 16, 17, 18, 19, or 20 carbons in length, or ranges including and/or spanning the aforementioned values) and glycerol. In some embodiments, the lipid component comprises a short chain triglyceride (SCT). In some embodiments, the short chain triglyceride comprises a fatty acid tail less than 6 carbons in length (e.g., less than or equal to 5, 4, 3, 2, 1 carbons in length, or ranges including and/or spanning the aforementioned values). In some embodiments, the lipid is a triglyceride that is a tri-ester of fatty acids having aliphatic chain lengths 6 to 20 carbons in length. In some embodiments, the composition lacks long chain triglycerides. In some embodiments, the lipid comprises one or more of tricaprin, trilaurin, trimyristine, tripalmitin, and tristearin. In some embodiments, the lipid is a triglyceride that is a tri-ester of fatty acids having aliphatic chain lengths 1 to 20 carbons in length. In some embodiments, the composition lacks long chain triglycerides.

In some embodiments, the lipid (e.g., that is not a phospholipid) comprises one or more short chain, medium chain, long chain fatty acids, or combinations thereof (e.g., non-triglycerides that are not an ester of glycerol). In some embodiments, the short chain fatty acid comprises a fatty acid tail less than 6 carbons in length (e.g., less than or equal to 5, 4, 3, 2, 1 carbons in length, or ranges including and/or spanning the aforementioned values). In several embodiments, the medium chain fatty acid comprises an aliphatic tail 6-12 carbons in length (e.g., 6, 7, 8, 9, 10, 11, or 12). In some embodiments, the long chain fatty acid comprises a tail that is greater than 12 carbons in length (e.g., greater than or equal to 13, 14, 15, 16, 17, 18, 19, or 20 carbons in length, or ranges including and/or spanning the aforementioned values). In some embodiments, the lipid comprises one or more different chain length fatty acids (short chain, medium chain, long chain, or combinations thereof). In some embodiments, the lipid comprises one or more different chain length fatty acids (short chain, medium chain, long chain, or combinations thereof) and one or more different chain length triglycerides (as disclosed elsewhere herein).

In some embodiments, the one or more lipid(s) (e.g., lipids that are not phospholipids, such as SCT(s), MCT(s), LCT(s), combinations thereof, etc.), collectively or individually, are present in the aqueous composition at a concentration of less than or equal to about: 400 mg/ml, 300 mg/ml, 200 mg/ml, 150 mg/ml, 100 mg/ml, 93 mg/ml, 75 mg/ml, 50 mg/ml, 25 mg/ml, or ranges including and/or spanning the aforementioned values. In some embodiments, the one or more lipids are present in the composition (collectively or individually) at a dry wt % of equal to or less than about: 2.5%, 5%, 10%, 15%, 20%, 30%, 40%, 50%, 60%, or ranges including and/or spanning the aforementioned values. In some embodiments, the one or more lipids (collectively or individually) are present in the composition at a wet wt % of equal to or less than about: 2.5%, 5%, 7.5%, 10%, 12.5%, 15%, 20%, 30%, 40%, or ranges including and/or spanning the aforementioned values. In some embodiments, as disclosed elsewhere herein, the composition is aqueous, while in others it has been dried into a powder. In some embodiments, the lipid has a purity of greater than or equal to about: 92.5%, 95%, 96%, 98%, 99%, 99.9%, or ranges including and/or spanning the aforementioned values. In some embodiments, the lipid has a total % impurity content by weight of less than or equal to about: 8.5%, 5%, 4%, 3.7%, 2%, 1%, 0%, or ranges including and/or spanning the aforementioned values.

In some embodiments, the lipid that is not a phospholipid is not an MCT or LCT but is an MCT-substitute. In some embodiments, the MCT-substitute lipid (e.g., the non-phospholipid lipid) is selected from one or more of oleic acid, capric acid, caprylic acid, and triglycerides of such (Captex 8000, Captex GTO, Captex 1000), glycerol monooleate, glycerol monostearate (Geleol™ Mono and Diglyceride NF), omega-3 fatty acids (α-linolenic acid (ALA), eicosapentaenoic acid (EPA), docosahexaenoic acid (DHA), Tonalin, Pronova Pure® 46:38, free fatty acid Tonalin FFA 80), conjugated linoleic acid, alpha glycerylphosphorylcholine (alpha GPC), palmitoylethanolamide (PEA), cetyl alcohol, or emulsifying wax. In some embodiments, the one or more MCT-substitute lipids are present in the lipid-based particle composition (collectively or individually) at a dry wt % of equal to or less than about: 0.5%, 1.0%, 2.5%, 5%, 10%, 15%, 20%, 30%, 40%, 50%, 60%, 80% or ranges including and/or spanning the aforementioned values. In some embodiments, the one or more MCT-substitute lipids (collectively or individually) are present in the composition at a wet wt % of equal to or less than about: 0.5%, 1.0% 2.5%, 5%, 7.5%, 10%, 12.5%, 15%, 20%, 30%, 40%, 60% or ranges including and/or spanning the aforementioned values. In some embodiments, the MCT-substitute lipid has a purity of greater than or equal to about: 70%, 80%, 85%, 92.5%, 95%, 96%, 98%, 99%, 99.9%, 100%, or ranges including and/or spanning the aforementioned values. In some embodiments, the MCT-substitute lipid has a total % impurity content by weight of less than or equal to about: 8.5%, 5%, 4%, 3.7%, 2%, 1%, 0%, or ranges including and/or spanning the aforementioned values.

In some embodiments, a lipid that, when mixed with the therapeutic agent (e.g., CBD) at a wt % of equal to or less than about: 1%, 2.5%, 5%, 7.5%, 10%, 15%, 18%, 20%, 25%, (or ranges including and/or spanning the aforementioned values), the therapeutic agent (e.g., CBD isolate) is soluble and stable for a period of less than about 30 days (e.g., has degradation of less than or equal to about: 0.5%, 1%, 2%, 10%, 15%, or ranges including and/or spanning the aforementioned values). In some embodiments, as disclosed elsewhere herein, the non-phospholipid lipid is an MCT.

### Preservatives

In some embodiments, the lipid-based particle composition comprises a preservative. In some embodiments, the preservative includes one or more benzoates (such as sodium benzoate or potassium benzoate), nitrites (such as sodium nitrite), sulfites (such as sulfur dioxide, sodium or potassium sulphite, bisulphite or metabisulphite), sorbates (such as sodium sorbate, potassium sorbate), ethylenediaminetetraacetic acid (EDTA) (and/or the disodium salt thereof), polyphosphates, organic acids (e.g., citric, succinic, malic, tartaric, benzoic, lactic and propionic acids), and/or antioxidants (e.g., vitamins such as vitamin E and/or vitamin C, butylated hydroxytoluene). In some embodiments, the one or more preservatives, collectively or individually, are present in the aqueous composition at a concentration of less than or equal to about: 10 mg/ml, 5 mg/ml, 1 mg/ml, 0.85 mg/ml, 0.5 mg/ml, 0.1 mg/ml, or ranges including and/or spanning the aforementioned values. In some embodiments, the one or more preservatives (collectively or individually) are present in the composition at a dry wt % of equal to or at less than about: 0.01%, 0.1%, 0.25%, 0.5%, 1%, 5%, 7.5%, 10%, 15%, 20%, 25%, or ranges including and/or spanning the aforementioned values. In some embodiments, the one or more preservatives (collectively or individually) are present in the composition at a wet wt % of equal to or less than about: 0.001%, 0.01%, 0.025%, 0.05%, 0.1%, 0.5%, 0.75%, 1.0%, 1.5%, 2.0%, 2.5%, 5%, or ranges including and/or spanning the aforementioned values. In some embodiments, as disclosed elsewhere herein, the composition is aqueous, while in others it has been dried into a powder. In some embodiments, the aqueous composition comprises one or more of malic acid at about 0.85 mg/ml, citric acid at about 0.85 mg/ml, potassium sorbate at about 1 mg/ml, and sodium benzoate at about 1 mg/ml. In some embodiments, the preservatives inhibit or prevent growth of mold, bacteria, and fungus. In some embodiments, Vitamin E is added at 0.5 mg/ml to act as an antioxidant in the oil phase. In some embodiments, the preservative concentrations may be changed depending on the flavored oil used.

### Flavoring Agents

In some embodiments, the lipid-based particle composition comprises one or more flavoring agents. In some embodiments, the one or more flavoring agent(s), collectively or individually, are present in the aqueous composition at a concentration of less than or equal to about: 10 mg/ml, 5 mg/ml, 1.5 mg/ml, 1.2 mg/ml, 1 mg/ml, 0.9 mg/ml, 0.5 mg/ml, 0.1 mg/ml, or ranges including and/or spanning the aforementioned values. In some embodiments, the one or more flavoring agent(s) (collectively or individually) are present in the composition at a dry wt % of equal to or less than about: 0.01%, 0.1%, 0.25%, 0.5%, 1%, 5%, 7.5%, 10%, 15%, 20%, 25%, or ranges including and/or spanning the aforementioned values. In some embodiments, the one or more flavoring agents (collectively or individually) are present in the composition at a wet wt % of equal to or less than about: 0.001%, 0.01%, 0.025%, 0.05%, 0.1%, 0.5%, 0.75%, 1.0%, 1.5%, 2.0%, 2.5%, 5.0%, 10%, or ranges including and/or spanning the aforementioned values. In some embodiments, as disclosed elsewhere herein, the composition is aqueous, while in others it has been dried into a powder. In some embodiments, the one or more flavoring agents of the composition comprise monk fruit extract (e.g., MonkGold50), stevia, peppermint oil, lemon oil, vanilla, or the like, or combinations thereof. In some embodiments, the composition contains MonkGold50 at 0.9 mg/ml and flavored oils as flavoring. Examples of flavored oils are peppermint and lemon at 1.2 mg/ml. Chemicals that are not oil may also be used for flavor, for example, such as dry powders that replicate a flavor such as vanilla.

### Water Content

In some embodiments, the lipid-based particle composition is aqueous while in other embodiments the composition may be provided as a dry or substantially dry solid (e.g., having a water content in weight % of less than or equal to 50%, 40%, 30%, 20%, 15%, 10%, 5%, 2%, 1%, 0.5%, or ranges including and/or spanning the aforementioned values). In some embodiments, where the lipid-based particle composition is aqueous, water may be present at a wet weight percent of equal to or less than about: 60%, 70%, 75%, 77%, 80%, 85%, 90%, 95%, 97.5%, 99%, or ranges including and/or spanning the aforementioned values.

### Carbohydrates and Other Additives

In some embodiments, the lipid-based particle composition comprises one or more carbohydrates (and/or a carbohydrate source). In several embodiments, the carbohydrate source is selected from the group consisting of trehalose, sucrose, dextrose, glucose, isomaltulose, tagatose, arabinose, maltose, fructose, dextrin, lactose, maltose, fucose, galactose, inositol, maltodextrin, maltol, mannose, muscovado, ribose, rhamnose, saccharose, sucralose, xylose, lecithin, avocado fiber, acacia fiber, psyllium fiber, beta-glucan, guar gum, xanthan gum, pectin, chitin, cellulose, hemicellulose, sodium alginate, potassium alginate, ammonium alginate, calcium alginate, ammonium alginate, propylene glycol alginate, agar, carrageen, raffinose, polydextrose, cyclodextrins, fullerene, inulin, gelatin, pentose, and combinations thereof. In some embodiments, the one or more carbohydrates (or carbohydrate source(s)), collectively or individually, are present in the aqueous composition at a concentration of less than or equal to about: 100 mg/ml, 90 mg/ml, 80 mg/ml, 70 mg/ml, 60 mg/ml, 50 mg/ml, 40 mg/ml, 30 mg/ml, 20 mg/ml, 10 mg/ml, 5 mg/ml, 1.5 mg/ml, 1.2 mg/ml, 1 mg/ml, 0.9 mg/ml, 0.5 mg/ml, 0.1 mg/ml, 0.01 mg/ml, 0.001 mg/ml or ranges including and/or spanning the aforementioned values. In some embodiments, the one or more carbohydrates (collectively or individually) are present in the composition at a dry wt % of equal to or less than about: 0.001%, 0.01%, 0.1%, 1%, 5%, 7.5%, 10%, 15%, 20%, 25%, 35%, 50%, 60%, 70%, 80%, 90%, 95% or ranges including and/or spanning the aforementioned values. In some embodiments, the one or more carbohydrates (collectively or individually) are present in the composition at a wet wt % of equal to or less than about: 0.001%, 0.01%, 0.1%, 0.1%, 0.5%, 0.75%, 1.0%, 1.5%, 2.0%, 2.5%, 5.0%, 10%, 15%, 20%, 30%, 40%, 50%, or ranges including and/or spanning the aforementioned values. In some embodiments, as disclosed elsewhere herein, the composition is aqueous, while in others it has been dried into a powder. In some embodiments, the one or more carbohydrates, surprisingly, stabilize the lipid composition when in powdered form (e.g., dry or substantially dry form). In some embodiments, the one or more carbohydrates surprisingly help the lipid composition to return to particle form (e.g., nano or microparticle form) when reconstituted.

In some embodiments, the lipid-based particle composition comprises one or more one or more other additives, such as amino acids, polyethylene glycols, etc. In some embodiments, the one or more additives (or carbohydrate source(s)), collectively or individually, are present in the aqueous composition at a concentration of less than or equal to about: 100 mg/ml, 90 mg/ml, 80 mg/ml, 70 mg/ml, 60 mg/ml, 50 mg/ml, 40 mg/ml, 30 mg/ml, 20 mg/ml, 10 mg/ml, 5 mg/ml, 1.5 mg/ml, 1.2 mg/ml, 1 mg/ml, 0.9 mg/ml, 0.5 mg/ml, 0.1 mg/ml, 0.01 mg/ml, 0.001 mg/ml or ranges including and/or spanning the aforementioned values. In some embodiments, the one or more additives (collectively or individually) are present in the composition at a dry wt % of equal to or less than about: 0.001%, 0.01%, 0.1%, 1%, 5%, 7.5%, 10%, 15%, 20%, 25%, 35%, 50%, 60%, 70%, 80%, 90%, 95% or ranges including and/or spanning the aforementioned values. In some embodiments, the one or more additives (collectively or individually) are present in the composition at a wet wt % of equal to or less than about: 0.001%, 0.01%, 0.1%, 0.1%, 0.5%, 0.75%, 1.0%, 1.5%, 2.0%, 2.5%, 5.0%, 10%, 15%, 20%, 30%, 40%, 50%, or ranges including and/or spanning the aforementioned values. In some embodiments, as disclosed elsewhere herein, the composition is aqueous, while in others it has been dried into a powder. In some embodiments, the one or more additives, surprisingly, stabilize the lipid composition when in powdered form (e.g., dry or substantially dry form). In some embodiments, the one or more additives surprisingly help the lipid composition to return to particle form (e.g., nano or microparticle form) when reconstituted.

### Exemplary Compositions and Methods

In some embodiments, the aqueous lipid-based particle composition comprises phosphatidylcholine in a range from about 8% to about 12%, MCT in a range from about 8% to about 12%, one or more therapeutic agents (e.g., CBD and/or others) in a range from about 1% to about 5%, cholesterol in a range from about 0.5% to about 4%, and water in a range from about 60% to about 90%. In some embodiments, the aqueous composition further comprises one or more of vitamin E in a range from about 0.01% to about 1.0%, malic acid in a range from about 0.01% to about 1.0%, citric acid in a range from about 0.01% to about 1.0%, potassium sorbate in a range from about 0.01% to about 2.0%, sodium benzoate in a range from about 0.01% to about 2.0%, and/or monk fruit extract in a range from about 0.01% to about 2.0%. In some embodiments, as disclosed elsewhere herein, the composition is aqueous and includes one or more therapeutic agents (e.g., CBD and/or others) at about 20 mg/ml, phosphatidylcholine at about 100 mg/ml, cholesterol at about 10 mg/ml, and MCT at about 93 mg/ml.

In some embodiments, the aqueous lipid-based particle composition comprises phosphatidylcholine in a range from about 9% to about 11%, MCT in a range from about 8% to about 10%, one or more therapeutic agents (e.g., CBD and/or others) in a range from about 1% to about 3%, cholesterol in a range from about 0.5% to about 2%, and water in a range from about 70% to about 80%. In some embodiments, the aqueous composition further comprises one or more of vitamin E in a range from about 0.01% to about 1.0%, malic acid in a range from about 0.01% to about 1.0%, citric acid in a range from about 0.01% to about 1.0%, potassium sorbate in a range from about 0.01% to about 2.0%, sodium benzoate in a range from about 0.01% to about 2.0%, and/or monk fruit extract in a range from about 0.01% to about 2.0%.

In some embodiments, the lipid-based particle composition comprises (in dry wt %) phosphatidylcholine in a range from about 40% to about 50%, MCT in a range from about 35% to about 45%, one or more therapeutic agents (e.g., CBD and/or others) in a range from about 5% to about 25%, and cholesterol in a range from about 2.5% to about 10%. In some embodiments, the composition further comprises (in dry weight) one or more of vitamin E in a range from about 0.01% to about 2.0%, malic acid in a range from about 0.01% to about 2.0%, citric acid in a range from about 0.01% to about 2.0%, potassium sorbate in a range from about 0.01% to about 2.0%, sodium benzoate in a range from about 0.01% to about 2.0%, and/or monk fruit extract in a range from about 0.01% to about 2.0%.

In some embodiments, the lipid-based particle composition comprises (in dry wt %) phosphatidylcholine in a range from about 42% to about 46%, MCT in a range from about 39% to about 43%, one or more therapeutic agents (e.g., CBD and/or others) in a range from about 5% to about 15%, and cholesterol in a range from about 2.5% to about 7%. In some embodiments, the composition further comprises (in dry weight) one or more of vitamin E in a range from about 0.01% to about 2.0%, malic acid in a range from about 0.01% to about 2.0%, citric acid in a range from about 0.01% to about 2.0%, potassium sorbate in a range from about 0.01% to about 2.0%, sodium benzoate in a range from about 0.01% to about 2.0%, and/or monk fruit extract in a range from about 0.01% to about 2.0%. As disclosed elsewhere herein, the composition can be varied such that the different ratios of the components yield a nanoparticle containing one or more therapeutic agents (e.g., CBD and/or others) that are stable.

In some embodiments, a solid lipid nanoparticle of the lipid-based particle compositions comprises a lipid core matrix. In some embodiments, the lipid core matrix is solid. In some embodiments, the solid lipid comprises one or more ingredients as disclosed elsewhere herein. In some embodiments, the core of the solid lipid comprises one or more triglycerides (e.g., tristearin), diglycerides (e.g. glycerol behenate), monoglycerides (e.g. glycerol monostearate), fatty acids (e.g. stearic acid), steroids (e.g. cholesterol), and waxes (e.g. cetyl palmitate). In some embodiments, emulsifiers can be used to stabilize the lipid dispersion (with respect to charge and molecular weight). In some embodiments, the core ingredients and/or the emulsifiers are present in the composition (collectively or individually) at a dry wt % of equal to or less than about: 0.5%, 1.0%, 2.5%, 5%, 10%, 15%, 20%, 30%, 40%, 50%, 60%, 80% or ranges including and/or spanning the aforementioned values. In some embodiments, the core ingredients and/or the emulsifiers (collectively or individually) are present in the composition at a wet wt % of equal to or less than about: 0.5%, 1.0% 2.5%, 5%, 7.5%, 10%, 12.5%, 15%, 20%, 30%, 40%, 60% or ranges including and/or spanning the aforementioned values. In some embodiments, the core ingredients and/or the emulsifiers have a purity of greater than or equal to about: 70%, 80%, 85%, 92.5%, 95%, 96%, 98%, 99%, 99.9%, 100%, or ranges including and/or spanning the aforementioned values.

In some embodiments, the lipid-based particle composition (e.g., when in water or dried) comprises multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In some embodiments, the composition is characterized by having multiple types of particles (e.g., lamellar, emulsion, irregular, etc.). In other embodiments, a majority of the particles present are emulsion particles. In several embodiments, a majority of the particles present are lamellar (multilamellar and/or unilamellar). In other embodiments, a majority of the particles present are irregular particles. In still other embodiments, a minority of the particles present are emulsion particles. In some embodiments, a minority of the particles present are lamellar (multilamellar and/or unilamellar). In other embodiments, a minority of the particles present are irregular particles.

In some embodiments, of the particles present in the composition (e.g., the aqueous composition), equal to or at least about 5%, 8%, 9%, 10%, 15%, 25%, 50%, 75%, 85%, 95%, or 100% (or ranges spanning and/or including the aforementioned values) are multilamellar nanoparticle vesicles. In some embodiments, of the particles present in the composition (e.g., the aqueous composition), equal to or at least about 5%, 8%, 9%, 10%, or 15% (or ranges spanning and/or including the aforementioned values) are multilamellar nanoparticle vesicles. For example, in some embodiments, between about 5% and about 10% of the particles present are multilamellar. In some embodiments, about 8.6% of the particles present are multilamellar.

In some embodiments, of the particles present in the composition (e.g., the aqueous composition), equal to or at least about 5%, 8%, 9%, 10%, 15%, 25%, 50%, 75%, 85%, 95%, or 100% (or ranges spanning and/or including the aforementioned values) are unilamellar nanoparticle vesicles. In some embodiments, of the particles present in the composition (e.g., the aqueous composition), equal to or at least about 5%, 8%, 9%, 10%, 15%, or 20% (or ranges spanning and/or including the aforementioned values) are unilamellar nanoparticle vesicles. For example, in some embodiments, between about 10% and about 15% of the particles present are unilamellar. In some embodiments, about 12.88% of the particles present are unilamellar.

In some embodiments, of the particles present in the composition (e.g., the aqueous composition), equal to or at least about 5%, 8%, 9%, 10%, 15%, 25%, 50%, 75%, 85%, 95%, or 100% (or ranges spanning and/or including the aforementioned values) are emulsion particles. In some embodiments, of the particles present in the composition (e.g., the aqueous composition), equal to or at least about 60%, 65%, 70%, 75%, 85%, 95%, or 100% (or ranges spanning and/or including the aforementioned values) are emulsion particles. For example, in some embodiments, between about 60% to about 75% of the particles present are emulsion particles. In some embodiments, about 69.7% of the particles present are emulsion particles.

In some embodiments, of the particles present in the composition (e.g., the aqueous composition), equal to or at least about 1%, 2%, 3%, 5%, 8%, 9%, 10%, 15%, 25%, 50%, 75%, 85%, 95%, or 100% (or ranges spanning and/or including the aforementioned values) are irregular particles (e.g., with lamellar structures and/or bridges). In some embodiments, of the particles present in the composition (e.g., the aqueous composition), equal to or at least about 1%, 2%, 3%, 5%, 8%, 9%, or 10% (or ranges spanning and/or including the aforementioned values) are irregular particles. For example, in some embodiments, between about 1% to about 5% of the particles present are irregular particles. In some embodiments, 2.73% are irregular particles.

In some embodiments, of the particles present in the composition (e.g., the aqueous composition), equal to or at least about 5%, 8%, 9%, 10%, 15%, 25%, 50%, 75%, 85%, 95%, or 100% (or ranges spanning and/or including the aforementioned values) are combined lamellar and emulsion particles. In some embodiments, of the particles present in the composition (e.g., the aqueous composition), equal to or at least about 5%, 8%, or 9% (or ranges spanning and/or including the aforementioned values) are combined lamellar and emulsion particles. For example, in some embodiments, between about 5% to about 6% of the particles present are combined lamellar and emulsion particles. In some embodiments, 6.06% of the particles are combined lamellar and emulsion particles.

In some embodiments, the composition (e.g., the aqueous composition) comprises between 60% and 80% emulsion particles. In some embodiments, the composition (e.g., the aqueous composition) comprises between 7.5% and 20% small unilamellar vesicles. In some embodiments, the composition (e.g., the aqueous composition) comprises between 5% and 15% multilamellar vesicles. In some embodiments, the composition (e.g., the aqueous composition) comprises between 3% and 10% combined lamellar and emulsion particles. In some embodiments, the composition (e.g., the aqueous composition) comprises between 1% and 6% irregular particles. In some embodiments, the composition (e.g., the aqueous composition) comprises between 65% and 75% emulsion particles. In some embodiments, the composition (e.g., the aqueous composition) comprises between 10% and 15% small unilamellar vesicles. In some embodiments, the composition (e.g., the aqueous composition) comprises between 5% and 12% multilamellar vesicles. In some embodiments, the composition (e.g., the aqueous composition) comprises between 4% and 8% combined lamellar and emulsion particles. In some embodiments, the composition (e.g., the aqueous composition) comprises between 1% and 4% irregular particles.

In some embodiments, the composition (e.g., the aqueous composition) comprises between 60% and 80% emulsion particles, between 7.5% and 20% small unilamellar vesicles, between 5% and 15% multilamellar vesicles, between 3% and 10% combined lamellar and emulsion particles, and between 1% and 6% irregular particles. In some embodiments, the composition (e.g., the aqueous composition) comprises between 65% and 75% emulsion particles, between 10% and 15% small unilamellar vesicles, between 5% and 12% multilamellar vesicles, between 4% and 8% combined lamellar and emulsion particles, and between 1% and 4% irregular particles. In some embodiments, the composition (e.g., the aqueous composition) comprises 69.7% emulsion particles, 12.88% small unilamellar vesicles, 8.64% multilamellar vesicles, 6.06% combined lamellar and emulsion particles, and 2.73% irregular particles.

In some embodiments, at ambient temperature an aqueous lipid-based composition as disclosed herein has a viscosity (in centipoise (cP)) of equal to or less than about: 1.0, 1.05, 1.1, 1.2, 1.5, 2.0, 5.0, 10.0, 20, 30, 50, 100, or ranges including and/or spanning the aforementioned values. In some embodiments, at about 25° C. or 26° C. and a concentration of 20 mg/ml active agent (e.g., CBD) in water (e.g., the total lipid-based particle composition may have a concentration of 229.4 to 235.6 mg/mL), the lipid-based particle composition has a viscosity (in centipoise (cP)) of equal to or less than about: 1.0, 1.05, 1.1, 1.2, 1.5, 2.0, 5.0, 10.0, 20, 30, 50, 100, or ranges including and/or spanning the aforementioned values. In some embodiments, at about 25° C. and a concentration of 229.4 to 235.6 mg/mL, the lipid-based particle composition has a viscosity (in cP) of equal to or less than about: 1.0, 1.05, 1.1, 1.2, 1.5, 2.0, 5.0, 10.0, 20, 30, 50, 100, or ranges including and/or spanning the aforementioned values. In some embodiments, the viscosity of the CBD lipid nanoparticle aqueous solution is equal to or less than 5.0 Cp.

In some embodiments, the liposomes and/or a liquid (e.g., aqueous) composition comprising the nanoparticles as disclosed herein are lyophilized. In some embodiments, where lyophilization is used to prepare a liposomal and/or nanoparticle based powder, one or more lyoprotectant agents may be added. In some embodiments, an individual lyoprotectant agent may be present at a dry wt % equal to or less than the dry weight of the lipophilic ingredients. In some embodiments, the lyoprotectant agent(s) (collectively or individually) may be present at a dry wt % equal to or less than about: 5%, 10%, 15%, 20%, 30%, 40%, 50%, 60%, or ranges including and/or spanning the aforementioned values. In some embodiments, the lyoprotectant agent(s) (collectively or individually) may be present at a wet wt % of equal to or less than about: 2.5%, 5%, 7.5%, 10%, 12.5%, 15%, 20%, 30%, or ranges including and/or spanning the aforementioned values. In some embodiments, the lyoprotectant is selected from the group consisting of lactose, dextrose, trehalose, arginine, glycine, histidine, and/or combinations thereof.

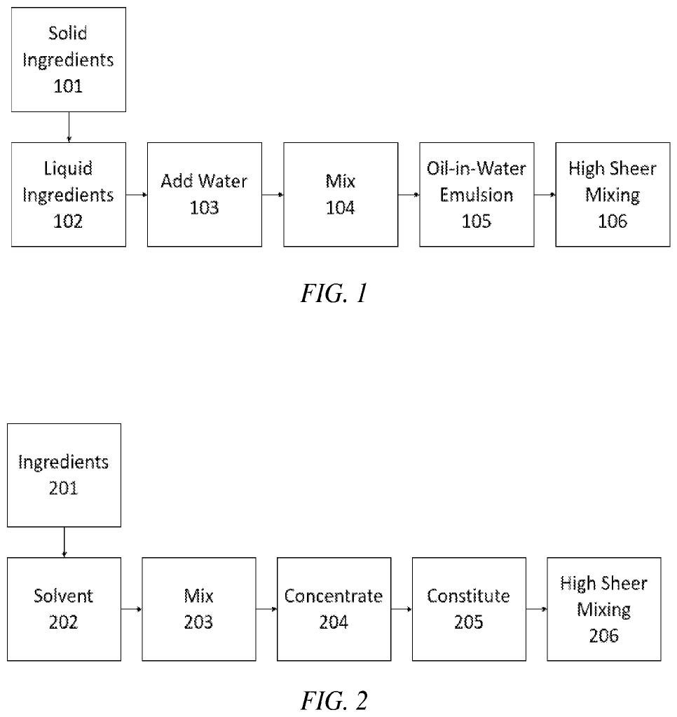

As disclosed elsewhere herein, some embodiments pertain to methods of preparing lipid-based particle compositions comprising nanoparticles and/or liposomes. In some embodiments, the composition is prepared by forming a lipid-in-oil emulsion. In some embodiments, an oil-in-water emulsion can be prepared without the use of organic solvents as shown in FIG. 1 (e.g., in an organic solvent-free method). In some embodiments, solid ingredients 101 are added and dissolved into liquid ingredients 102. In some embodiments, for example, one or more of the sterol (e.g., cholesterol) and/or therapeutic agent(s) (e.g., phytocannabinoid, CBD, etc.) can be dissolved in lipid oil (e.g., a medium chain triglyceride) and/or vitamin E. In some embodiments, the phospholipid (e.g., phosphatidylcholine) can be added with mixing. In some embodiments, when a well dispersed lipid phase is formed after mixing, the addition of water 103 (e.g., having a temperature of equal to or at least about: 10° C., 20° C., 30° C., 40° C., 50° C., 60° C., 80° C., or ranges including and/or spanning the aforementioned values) and additional mixing 104 achieves an oil-in-water emulsion 105. In some embodiments, the oil-in-water emulsion is then subject to high-shear mixing to form nanoparticles (e.g., therapeutic agent and/or CBD liposomes). In some embodiments, high-shear mixing 106 is performed using a high shear dispersion unit or an in-line mixer can be used to prepare the emulsions. In some embodiments, the particles can be made by solvent evaporation and/or solvent precipitation.

In some embodiments, as shown in FIG. 2, the lipid-in-oil emulsion is formed by dissolving ingredients 201, such as, one or more of a phospholipid (e.g., phosphatidylcholine), a sterol (e.g., cholesterol), one or more therapeutic agents (e.g., phytocannabinoid, CBD, etc.), a lipid (e.g., a medium chain triglyceride), and/or a preservative (e.g., vitamin E) in a solvent 202. In some embodiments, the solvent can include one or more organic solvents, including but not limited to, ethanol, chloroform, and/or ethyl acetate. In some embodiments, the solvents are class II solvents, class III solvents (e.g., at least class II and/or class III by the ICH Q3C standard), or mixtures thereof. In some embodiments, the solution of ingredients and solvent is dried 203. In some embodiments, after drying, the ingredients are provided as lipids and or liposomes as a thin film. In some embodiments, the solvent is removed from the composition by subjecting the solution to heat under vacuum to promote evaporation. In some embodiments, the film may further be dried under nitrogen gas. In some embodiments, the lipid film is hydrated 205 with warm aqueous solution to form an oil-in-water emulsion. In some embodiments, high-shear mixing is performed 206 using a high shear dispersion unit or an in-line mixer can be used to prepare the emulsions.

In some embodiments, as disclosed elsewhere herein, the lipid-in-water emulsion is subject to high pressure homogenization using a microfluidizer. In some embodiments, high sheer mixing can be used to reduce the particle size. In some embodiments, the oil-in-water emulsion is processed to a nanoparticle (e.g., about 20 to about 500 nm, etc.) using the microfluidizer or other high sheer processes. In some embodiments, the oil-in-water emulsion is processed to a nanoparticle having a size from about 80 nm to 180 nm in diameter or about 100 nm to about 150 nm in diameter.

In some embodiments, the lipid-in-water emulsion is passed through the microfluidizer a plurality of times (e.g., equal to or at least 1 time, 2 times, 3 times, 4 times, 5 times, 10 times, or ranges including and/or spanning the aforementioned values). In some embodiments, the emulsion is passed through the microfluidizer at a pressure of equal to or less than about: 5,000 PSI, 15,000 PSI, 20,000 PSI, 25,000 PSI, 30,000 PSI, or ranges including and/or spanning the aforementioned values. In some embodiments, the emulsion is passed through the microfluidizer at a temperature of equal to or at least about: 30° C., 40° C., 50° C., 65° C., 80° C., or ranges including and/or spanning the aforementioned values. In some embodiments, the emulsion is passed through the microfluidizer at least about room temperature (e.g., about 20° C. or about 25° C.) and/or without any heating and/or temperature control. In some embodiments, the emulsion is passed through the microfluidizer at a temperature of equal to or less than about 80° C. In some embodiments, the microfluidizer includes an interaction chamber consisting of 75 μm to 200 μm pore sizes and the emulsion is passed through this chamber. In some embodiments, the pore size of the microfluidizer are less than or equal to about: 75 μm, 100 μm, 150 μm, 200 μm, 250 μm, 300 μm, or ranges including and/or spanning the aforementioned values. In some embodiments, the nanoparticle composition is prepared by high shear mixing, sonication, or extrusion.

In some embodiments, after preparation, the lipid-based particle composition is characterized by an ability to pass through a 0.2 μm filter while preserving the nanoparticle structure (e.g., a change in average nanoparticle size of no greater than 10 nm, 20 nm, or 30 nm). In some embodiments, after passage through a 0.2 μm there is a change in average diameter of the particles of equal to or at less than about: 1%, 5%, 10%, 20%, or ranges including and/or spanning the aforementioned values. In some embodiments, after passage through a 0.2 μm there is a change in PDI of the particles of equal to or at less than about: 1%, 5%, 10%, 20%, or ranges including and/or spanning the aforementioned values.

In some embodiments, as disclosed elsewhere herein, the lipid-based particle composition is composed of highly pure ingredients. These may include GMP manufactured CBD isolate or other therapeutic agents. In some embodiments, the CBD or other therapeutic agents are triple checked for potency and purity, and has negligible concentrations of THC or other impurities. In some embodiments, the composition (and/or one or more ingredients constituting the compositions) is manufactured with high purity, multicompendial ingredients to be at the same standards as pharmaceutical products. In some embodiments, the composition is manufactured using pharmaceutical equipment and documentation to ensure the product is of high quality and consistent from batch to batch.

In some embodiments, as disclosed elsewhere herein, the therapeutic nanoparticle composition imparts solubility to hydrophobic therapeutic agents (e.g., CBD, other phytocannabinoids, etc.) in a delivery system that is easily dispersible in aqueous solutions. For example, CBD oils do not disperse well in aqueous solutions and have poor oral absorption. CBD particle formulations made using methods other than those disclosed herein have inconsistent particle size and may not be stable with storage over time.

In some embodiments, advantageously, the nanoparticle delivery systems (comprising e.g., cannabinoids, such as CBD, non-cannabinoids, and/or combinations of any of the foregoing) disclosed herein are reproducibly manufacturable. In some embodiments, the method of manufacture of the compositions avoids the introduction of contaminants (such as metal contamination). In some embodiments, over 50%, 75%, 95% (or ranges spanning and or including the aforementioned values) of the nanoparticles prepared by the methods disclosed herein have a particle size of between about 20 to about 500 nm (as measured by zeta sizing (e.g., refractive index). In some embodiments, over 50%, 75%, 95% (or ranges spanning and or including the aforementioned values) of the nanoparticles prepared by the methods disclosed herein have a particle size of between about 50 nm to about 200 nm (as measured by zeta sizing (e.g., refractive index). In some embodiments, over 50%, 75%, 95% (or ranges spanning and or including the aforementioned values) of the nanoparticles prepared by the methods disclosed herein have a particle size of between about 90 nm to about 150 nm (as measured by zeta sizing (e.g., refractive index). In some embodiments, this consistency in size allows predictable delivery to subjects. In some embodiments, the D90 particle size measurement varies between 150 and 500 nm.

In some embodiments, the lipid-based delivery system described herein offers protection to therapeutic agents (e.g., cannabinoids, such as CBD, non-cannabinoids, and/or combinations of any of the foregoing) against degradation in an aqueous environment for long-term storage. In some embodiments, the composition is well characterized to ensure a consistent product from batch to batch and with long-term stability. In some embodiments, the product stability is routinely tested for appearance, particle size and distribution, zeta potential, residual solvents, heavy metals, therapeutic agent (e.g., cannabinoids, such as CBD, non-cannabinoids, and/or combinations of any of the foregoing) concentration, and microbial testing and the values measured using these test methods varies (over a period of at least about 1 month or about 6 months at 25° C. with 60% relative humidity) by less than or equal to about: 1%, 5%, 10%, 20%, 30%, or ranges including and/or spanning the aforementioned values. In some embodiments, the particle size and/or PDI varies over a period of at least about 1 month or about 6 months (at 25° C. with 60% relative humidity) by less than or equal to about: 1%, 5%, 10%, 20%, 30%, or ranges including and/or spanning the aforementioned values. As noted elsewhere herein, PDI and size can be measured using conventional techniques disclosed herein. In some embodiments, the therapeutic agent (e.g., cannabinoids, such as CBD, non-cannabinoids, and/or combinations of any of the foregoing) concentration varies over a period of at least about 1 month or about 6 months (at 25° C. with 60% relative humidity) by less than or equal to about: 1%, 5%, 10%, 15%, or ranges including and/or spanning the aforementioned values. As noted elsewhere herein, PDI and size can be measured using conventional techniques disclosed herein.

In some embodiments, the formulations and/or compositions disclosed herein are stable during sterilization. In some embodiments, the sterilization may include one or more of ozonation, UV treatment, and/or heat treatment. In some embodiments, the particle size and/or PDI after sterilization (e.g., exposure to techniques that allow sterilization of the composition) varies by less than or equal to about: 1%, 5%, 10%, 20%, 30%, or ranges including and/or spanning the aforementioned values. In some embodiments, the therapeutic agent (e.g., cannabinoids, such as CBD, non-cannabinoids, and/or combinations of any of the foregoing) concentration after sterilization (e.g., exposure to techniques that allow sterilization of the composition) varies (e.g., drops) by less than or equal to about: 1%, 5%, 10%, 15%, or ranges including and/or spanning the aforementioned values.

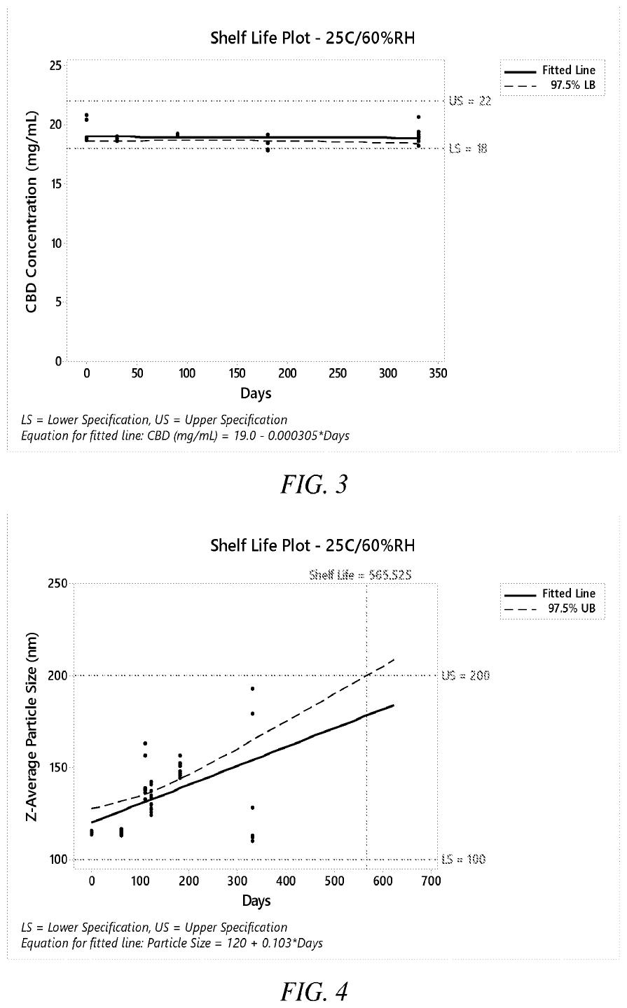

In some embodiments, the lipid-based particle compositions (including after stabilization) disclosed herein have a shelf life of equal to or greater than 6 months, 12 months, 14 months, 16 months, 18 months, 19 months, or ranges including and/or spanning the aforementioned values. The shelf-life can be determined as the period of time in which there is 95% confidence that at least 50% of the response (therapeutic agent(s) concentration or particle size) is within the specification limit. This refers to a 95% confidence interval and when linear regression predicts that at least 50% of the response is within the set specification limit. For instance, in FIG. 3, the dashed line on the stability plot is the 95% confidence interval and the solid line is the linear regression. The dots are the responses. The response variable is either Z-average particle size or therapeutic agent (e.g., cannabinoids, such as CBD, non-cannabinoids, and/or combinations of any of the foregoing) concentration in FIGS. 3 and 4. In some embodiments, the particle size specification is 100 to 200 nm, the therapeutic agent (e.g., cannabinoids, such as CBD, non-cannabinoids, and/or combinations of any of the foregoing) concentration specification is 18 to 22 mg/mL. These are shown on the stability plot as the lower specification (LS) and the upper specification (US).

In some embodiments, the lipid-based particle composition contains preservatives to protect against bacteria, mold, and fungal growth. The product specification is no more than 100 cfu/gram. In some embodiments, over a period of about 1 month, about 6 months, or about 12 months the composition has equal to or not more than: 50 cfu/gram, 10 cfu/gram, 5 cfu/gram, 1 cfu/gram, 0.1 cfu/gram, or ranges including and/or spanning the aforementioned values. In some embodiments, 1 week at 20° C.-25° C. after a 10⁵-10⁷ CFU/mL challenge with any one of Staphylococcus aureus, Pseudomonas aeruginosa, Escherichia coli, Candida albicans, and Aspergillus brasiliensis the composition has equal to or not more than: 100 cfu/gram, 50 cfu/gram, 25 cfu/gram, 10 cfu/gram, 5 cfu/gram, 1 cfu/gram, 0.1 cfu/gram, or ranges including and/or spanning the aforementioned values. In some embodiments, 1 week at 20° C.-25° C. after a 10⁵-10⁷ CFU/mL challenge with any one of Staphylococcus aureus, Pseudomonas aeruginosa, Escherichia coli, Candida albicans, and Aspergillus brasiliensis the composition has a log reduction for the bacteria of equal to or greater than: 1, 2, 3, 4, 5, 10, or ranges including and/or spanning the aforementioned values.

In some embodiments, unlike other delivery systems, the lipid-based particle composition ingredients provided herein provides a proper ratio and/or combination of ingredients that allow it to maintain stability and efficacy as disclosed elsewhere herein (e.g., during long term storage for example).

In some embodiments, advantageously, the individual particles within the disclosed lipid-based particle compositions may not settle or sediment appreciably. In some embodiments, an appreciable amount of the composition (e.g., as viewed by the naked eye) does not settle and/or separate from an aqueous liquid upon standing. In some embodiments, the composition does not appreciably settle or separate from an aqueous liquid upon standing for equal to or at least about 1 day, at least about 1 month, about 3 months, about 6 months, about 9 months, about 1 year, or ranges including and/or spanning the aforementioned values. In some embodiments, upon standing, the composition remains dispersed in an aqueous liquid for at least about 1 day, at least about 1 month, about 3 months, about 6 months, about 9 months, about 1 year, or ranges including and/or spanning the aforementioned values. In some embodiments, the homogeneity of the disclosed compositions changes by equal to or less than about: 0.5%, 1%, 5%, 7.5%, 10%, or 15% (or ranges including and/or spanning the aforementioned values) after a period of one week or one month. In this case, homogeneity is observed through images by SEM or cyro-SEM (e.g., the average size of the particles and/or the particle types). In some embodiments, the composition remains dispersed in an aqueous liquid and does not appreciably settle or separate from an aqueous liquid after at least about: 1 minute, 5 minutes, 30 minutes, or an hour in a centrifuge at a centripetal acceleration of at least about 100 m/s, at least about 1000 m/s, or at least about 10,000 m/s. In some embodiments, the composition remains dispersed in an aqueous liquid and does not appreciably settle or separate from an aqueous liquid after at least about: 1 minute, 5 minutes, 30 minutes, or an hour in a centrifuge at a centrifuge speed of 5000 RPM, 10,000 RPM, or 15,000 RPM.

In some embodiments, as disclosed elsewhere herein, the nanoparticle delivery system aids in absorption of the therapeutic agent (e.g., cannabinoids, such as CBD, non-cannabinoids, and/or combinations of any of the foregoing) molecule when orally ingested. In some embodiments, the compositions disclosed herein allow the therapeutic agent (e.g., cannabinoids, such as CBD, non-cannabinoids, and/or combinations of any of the foregoing) to be delivered to and/or absorbed through the gut. As disclosed elsewhere herein, some embodiments pertain to the use of the lipid-based nanodelivery system to protect the therapeutic agent (e.g., cannabinoids, such as CBD, non-cannabinoids, and/or combinations of any of the foregoing) from degradation and/or precipitation in the aqueous solution it is stored in (e.g., in an aqueous composition for administration to a subject). In some embodiments, use of the delivery systems disclosed herein result in improved bioavailability and/or absorption rate. For instance, in some embodiments, the Cmax of a therapeutic is increased using a disclosed embodiment, the Tmax of is decreased using an embodiment as disclosed herein, and/or the AUC is increased using a disclosed embodiment.

In some embodiments, the pharmacokinetic outcomes disclosed elsewhere herein (Cmax, Tmax, AUC, t_{1/2}, etc.) can be achieved using aqueous lipid-based particle compositions or powdered lipid-based particle compositions (e.g., where the powder is supplied by itself, in a gel capsule, as an additive to food, etc.).

In some embodiments, the Cmax of the therapeutic agent (e.g., CBD) is increased using the disclosed embodiments relative to other delivery vehicles (e.g., after administration to a subject). In some embodiments, the Cmax is increased relative to the therapeutic agent (e.g., cannabinoids, such as CBD, non-cannabinoids, and/or combinations of any of the foregoing) alone or comparator embodiments (e.g., oil-based products) by equal to or at least about: 15%, 20%, 50%, 100%, 150%, 200%, or ranges including and/or spanning the aforementioned values. In some embodiments, the therapeutic agent (e.g., cannabinoids, such as CBD, non-cannabinoids, and/or combinations of any of the foregoing) Cmax is increased (relative to a comparator oil-based product) by equal to or at least about: 5%, 10%, 20%, 30%, 50%, 100%, or ranges including and/or spanning the aforementioned values. In some embodiments, the therapeutic agent (e.g., cannabinoids, such as CBD, non-cannabinoids, and/or combinations of any of the foregoing) Cmax is increased (relative to a comparator oil-based product) by equal to or at least about: 10 ng/mL, 20 ng/mL, 30 ng/mL, 40 ng/mL, 50 ng/mL, 60 ng/mL, 70 ng/mL, 80 ng/mL, 90 ng/mL, or ranges including and/or spanning the aforementioned values.

In some embodiments, after a dose of 15 mg CBD provided in an embodiment as disclosed herein to a subject (e.g., a mini-pig, human, etc.), the Cmax of CBD is equal to or at least about: 0.5 μg/L, 1 μg/L, 2 μg/L, 3 μg/L, 4 μg/L, 5 μg/L, 6 μg/L, or ranges including and/or spanning the aforementioned values. In some embodiments, after a dose of 15 mg/kg of CBD provided in an embodiment as disclosed herein to a subject, the Cmax of CBD is equal to or at least about: 40 ng/mL, 50 ng/mL, 60 ng/mL, 70 ng/mL, 80 ng/mL, 90 ng/mL, 100 ng/mL, 150 ng/mL, 200 ng/mL, or ranges including and/or spanning the aforementioned values.

In some embodiments, after a dose of 15 mg therapeutic agent (e.g., cannabinoids, such as CBD, non-cannabinoids, and/or combinations of any of the foregoing) provided in an embodiment as disclosed herein to a subject (e.g., a mini-pig, human, etc.), the Cmax of the therapeutic agent is equal to or at least about: 0.5 μg/L, 1 μg/L, 2 μg/L, 3 μg/L, 4 μg/L, 5 μg/L, 6 μg/L, or ranges including and/or spanning the aforementioned values. In some embodiments, after a dose of 15 mg/kg of therapeutic agent (e.g., cannabinoids, such as CBD, non-cannabinoids, and/or combinations of any of the foregoing) provided in an embodiment as disclosed herein to a subject, the Cmax is equal to or at least about: 40 ng/mL, 50 ng/mL, 60 ng/mL, 70 ng/mL, 80 ng/mL, 90 ng/mL, 100 ng/mL, 150 ng/mL, 200 ng/mL, or ranges including and/or spanning the aforementioned values.

In some embodiments, the Cmax for a disclosed embodiment is increased relative to an equal dose of a therapeutic agent (e.g., cannabinoids, such as CBD, non-cannabinoids, and/or combinations of any of the foregoing) in an oil-based comparator vehicle. In some embodiments, the Cmax for a disclosed embodiment is increased relative to an oil-based comparator vehicle by equal to or at least about: 15%, 20%, 50%, 100%, 150%, 200%, or ranges including and/or spanning the aforementioned values. In some embodiments, these pharmacokinetic results can be achieved using aqueous compositions or powdered compositions (where the powder is supplied by itself, in a gel capsule, as an additive to food, etc.). In some instances, the Cmax using a disclosed embodiment is 1.25 times higher than when using a comparator delivery system (e.g., the Cmax of the comparator×1.25). In some instances, the Cmax using a disclosed embodiment is equal to or at least about 1.25 times higher, 1.5 times higher, 2 times higher, 3 times higher (or ranges including or spanning the aforementioned values) than when using a comparator delivery system.

In some embodiments, the Tmax for a therapeutic agent (e.g., cannabinoids, such as CBD, non-cannabinoids, and/or combinations of any of the foregoing) using a disclosed embodiment is shortened relative to other vehicles. In some embodiments, after a dose of therapeutic agent (e.g., cannabinoids, such as CBD, non-cannabinoids, and/or combinations of any of the foregoing) provided in an embodiment as disclosed herein to a subject as disclosed herein, the Tmax is equal to or at less than about: 30 minutes, 1 hours, 2 hours, 3 hours, 4 hours, 4.5 hours, 5 hours, 5.5 hours, 6 hours, 6.5 hours, 7 hours, 8 hours, or ranges including and/or spanning the aforementioned values. In some embodiments, after a dose of 15 mg/kg of therapeutic agent (e.g., cannabinoids, such as CBD, non-cannabinoids, and/or combinations of any of the foregoing) provided in an embodiment as disclosed herein to a subject, the Tmax is equal to or at less than about: 30 minutes, 1 hours, 2 hours, 3 hours, 4 hours, 5 hours, 5.5 hours, 6 hours, 6.5 hours, 7 hours, 8 hours, or ranges including and/or spanning the aforementioned values. In some embodiments, after a dose of therapeutic agent (e.g., cannabinoids, such as CBD, non-cannabinoids, and/or combinations of any of the foregoing) provided in an embodiment as disclosed herein to a subject, the Tmax is between about 4 hours and about 6.5 hours or between about 3 hours and about 7 hours. In some embodiments, after a dose of 15 mg of therapeutic agent (e.g., cannabinoids, such as CBD, non-cannabinoids, and/or combinations of any of the foregoing) provided in an embodiment as disclosed herein to a human patient, the Tmax is equal to or less than about: 3 hours, 4 hours, 5 hours, 6 hours, 7 hours, 8 hours, or ranges including and/or spanning the aforementioned values.

In some embodiments, the Tmax for the therapeutic agent (e.g., cannabinoids, such as CBD, non-cannabinoids, and/or combinations of any of the foregoing) using a disclosed embodiment is improved relative to oil-based vehicles (e.g., has a shorter duration to Tmax). In some embodiments, using an embodiment as disclosed herein, the Tmax is shortened relative to comparable delivery vehicles (e.g., an oil-based vehicle) by equal to or at least about: 5%, 10%, 15%, 20%, 25%, 50%, or ranges including and/or spanning the aforementioned values. In some embodiments, the Tmax is shortened relative to the therapeutic agent(s) alone by equal to or at least about: 5%, 10%, 15%, 20%, 25%, or ranges including and/or spanning the aforementioned values. In some embodiments, the Tmax for a disclosed embodiment is decreased relative to a oil-based comparator vehicle by equal to or at least about: 15%, 20%, 50%, 100%, 150%, 200%, or ranges including and/or spanning the aforementioned values. In some embodiments, the Tmax of a therapeutic agent (e.g., cannabinoids, such as CBD, non-cannabinoids, and/or combinations of any of the foregoing) for a disclosed embodiment is decreased relative to an oil-based comparator vehicle by equal to or at least about: 15 minutes, 30 minutes, 45 minutes, 1 hour, 2 hours, or ranges including and/or spanning the aforementioned values. In some instances, the Tmax is a fraction of that achieved using a comparator delivery system. In some instances, the time to Tmax using a disclosed embodiment is 0.5 times, 0.7 times, 0.8 times, 0.9 times, or 0.95 times the Tmax of a comparator delivery system (or ranges including or spanning the aforementioned values).

In some embodiments, after of a dose of therapeutic agent (e.g., cannabinoids, such as CBD, non-cannabinoids, and/or combinations of any of the foregoing) (e.g., a 15 mg/kg dose) provided in an embodiment as disclosed herein to a subject (e.g., a mini-pig, human, etc.), the AUC is equal to or at least about: 50 ng/mL*hr, 100 ng/mL*hr, 200 ng/mL*hr, 300 ng/mL*hr, 400 ng/mL*hr, 450 ng/mL*hr, 500 ng/mL*hr, 550 ng/mL*hr, 600 ng/mL*hr, 650 ng/mL*hr, 700 ng/mL*hr, 800 ng/mL*hr, 1000 ng/mL*hr, or ranges including and/or spanning the aforementioned values.

In some embodiments, the AUC for a therapeutic agent (e.g., cannabinoids, such as CBD, non-cannabinoids, and/or combinations of any of the foregoing) using a disclosed embodiment is increased (relative to a therapeutic agent itself or a comparator delivery vehicle) by equal to or at least about: 50 ng/mL*hr, 100 ng/mL*hr, 200 ng/mL*hr, 300 ng/mL*hr, 400 ng/mL*hr, or ranges including and/or spanning the aforementioned values. In some embodiments, the AUC using a disclosed embodiment is increased (relative to a therapeutic agent itself or a comparator delivery vehicle) by equal to or at least about: 5%, 10%, 20%, 30%, or ranges including and/or spanning the aforementioned values. In some embodiments, the AUC is improved relative to a therapeutic agent alone or in an oil mixture by equal to or at least about: 5%, 25%, 50%, 100%, 150%, 200%, or ranges including and/or spanning the aforementioned values. In some instances, the AUC using a disclosed embodiment is 1.25 times higher than when using a comparator delivery system. In some instances, the AUC using a disclosed embodiment is equal to or at least about 1.25 times higher, 1.5 times higher, 2 times higher, 3 times higher (or ranges including or spanning the aforementioned values) than when using a comparator delivery system.

In some embodiments, after of a dose of 15 mg/kg of therapeutic agent (e.g., cannabinoids, such as CBD, non-cannabinoids, and/or combinations of any of the foregoing) to a subject as disclosed herein, the AUC for the time period from administration to 4 hours post administration using a disclosed embodiment is equal to or at least about: 40 ng/mL*hr, 50 ng/mL*hr, 75 ng/mL*hr, 100 ng/mL*hr, 200 ng/mL*hr, 300 ng/mL*hr, 400 ng/mL*hr, 450 ng/mL*hr, or ranges including and/or spanning the aforementioned values. In some embodiments, after of a dose of 15 mg/kg of therapeutic agent (e.g., cannabinoids, such as CBD, non-cannabinoids, and/or combinations of any of the foregoing) to a subject, the AUC for the time period from administration to 4 hours post administration using a disclosed embodiment is increased (e.g., relative to the therapeutic alone or a comparator delivery vehicle) by equal to or at least about: 15 ng/mL*hr, 25 ng/mL*hr, 50 ng/mL*hr, 75 ng/mL*hr, or ranges including and/or spanning the aforementioned values. In some embodiments, after of a dose of 15 mg/kg of therapeutic agent (e.g., cannabinoids, such as CBD, non-cannabinoids, and/or combinations of any of the foregoing) to a subject, the AUC for the time period from administration to 4 hours post administration using a disclosed embodiment is increased (e.g., relative to the therapeutic alone or a comparator delivery vehicle) by equal to or at least about: 5%, 10%, 20%, 25%, 30%, 50%, 100%, 150%, 200%, or ranges including and/or spanning the aforementioned values. In some embodiments, the AUC for the time period from administration to 4 hours post administration using a disclosed embodiment is double that of a comparator delivery system, triple that of a comparator delivery system, quadruple that of a comparator delivery system, or higher.

In some embodiments, after of a dose of 15 mg/kg of therapeutic agent (e.g., cannabinoids, such as CBD, non-cannabinoids, and/or combinations of any of the foregoing) to a subject as disclosed herein, the AUC for the time period from 4 hours post administration to 6 hours post administration using a disclosed embodiment is equal to or at least about: 40 ng/mL*hr, 50 ng/mL*hr, 75 ng/mL*hr, 100 ng/mL*hr, 200 ng/mL*hr, 300 ng/mL*hr, 400 ng/mL*hr, 450 ng/mL*hr, or ranges including and/or spanning the aforementioned values. In some embodiments, after of a dose of 15 mg/kg of therapeutic agent (e.g., cannabinoids, such as CBD, non-cannabinoids, and/or combinations of any of the foregoing) to a subject, the AUC for the time period from 4 hours post administration to 6 hours post administration using a disclosed embodiment is increased (e.g., relative to the therapeutic alone or a comparator delivery vehicle) by equal to or at least about: 15 ng/mL*hr, 25 ng/mL*hr, 50 ng/mL*hr, 75 ng/mL*hr, or ranges including and/or spanning the aforementioned values. In some embodiments, after of a dose of 15 mg/kg of therapeutic agent (e.g., cannabinoids, such as CBD, non-cannabinoids, and/or combinations of any of the foregoing) to a subject, the AUC for the time period from 4 hours post administration to 6 hours post administration using a disclosed embodiment is increased (e.g., relative to the therapeutic alone or a comparator delivery vehicle) by equal to or at least about: 5%, 10%, 20%, 25%, 30%, 50%, 100%, 150%, 200%, or ranges including and/or spanning the aforementioned values. In some embodiments, the AUC for the time period from 4 hours post administration to 6 hours post administration using a disclosed embodiment is double that of a comparator delivery system, triple that of a comparator delivery system, quadruple that of a comparator delivery system, or higher.

In some embodiments, the half-life for a therapeutic agent (e.g., cannabinoids, such as CBD, non-cannabinoids, and/or combinations of any of the foregoing) (t_{1/2}) in vivo using a disclosed embodiment can be shorter relative to other vehicles. In some embodiments, after a dose of therapeutic agent (e.g., cannabinoids, such as CBD, non-cannabinoids, and/or combinations of any of the foregoing) provided in an embodiment as disclosed herein to a subject as disclosed herein, the t_{1/2} of therapeutic agent (e.g., cannabinoids, such as CBD, non-cannabinoids, and/or combinations of any of the foregoing) is equal to or at less than about: 4 hours, 5 hours, 5.5 hours, 6 hours, 6.5 hours, or ranges including and/or spanning the aforementioned values. In some embodiments, after a dose of therapeutic agent (e.g., cannabinoids, such as CBD, non-cannabinoids, and/or combinations of any of the foregoing) provided in an embodiment as disclosed herein to a subject, the t_{1/2} of therapeutic agent (e.g., cannabinoids, such as CBD, non-cannabinoids, and/or combinations of any of the foregoing) is between about 4 hours and about 6.5 hours or between about 3 hours and about 7 hours. In some embodiments, the t_{1/2} for a disclosed embodiment is decreased relative to a therapeutic agent alone or an oil-based comparator vehicle by equal to or at least about: 15%, 20%, 50%, 100%, 150%, 200%, or ranges including and/or spanning the aforementioned values. In some embodiments, the t_{1/2} of therapeutic agent (e.g., cannabinoids, such as CBD, non-cannabinoids, and/or combinations of any of the foregoing) for a disclosed embodiment is decreased relative to the therapeutic alone or an oil-based comparator vehicle by equal to or at least about: 15 minutes, 30 minutes, 45 minutes, 1 hour, 2 hours, or ranges including and/or spanning the aforementioned values. In some instances, the t_{1/2} is a fraction of that achieved using a comparator delivery system. In some instances, the time to t_{1/2} using a disclosed embodiment is 0.5 times, 0.7 times, 0.8 times, 0.9 times, or 0.95 times the t_{1/2} of a comparator delivery system (or ranges including or spanning the aforementioned values).

For brevity, in some places, the Cmax, Tmax, AUC, and t_{1/2} results provided are disclosed with specific reference to CBD as the active agent. The above pharmacokinetic results (including Cmax, Tmax, AUC, and t_{1/2}) are also expected for other phytocannabinoids and/or other therapeutic agents as disclosed elsewhere herein.

In some embodiments, the lipid-based particle composition comprises nanoparticles having an average size of less than or equal to about: 10 nm, 50 nm, 100 nm, 250 nm, 500 nm, 1000 nm, or ranges including and/or spanning the aforementioned values. In some embodiments, the composition comprises nanoparticles having an average size of between about 50 nm and 150 nm or between about 50 and about 250 nm. In some embodiments, the size distribution of the nanoparticles for at least 50%, 75%, 80%, 90% (or ranges including and/or spanning the aforementioned percentages) of the particles present is equal to or less than about: 20 nm, 40 nm, 60 nm, 80 nm, 100 nm, 110 nm, 120 nm, 130 nm, 140 nm, 160 nm, 180 nm, 200 nm, 300 nm, 400 nm, 500 nm, or ranges including and/or spanning the aforementioned nm values. In some embodiments, the composition comprises nanoparticles having an average size of less than or equal to about: 10 nm, 50 nm, 100 nm, 250 nm, 500 nm, 1000 nm, or ranges including and/or spanning the aforementioned values. In some embodiments, the size distribution of the nanoparticles for at least 90% of the particles present is equal to or less than about: 20 nm, 40 nm, 60 nm, 80 nm, 100 nm, 110 nm, 120 nm, 130 nm, 140 nm, 160 nm, 180 nm, 200 nm, 300 nm, 400 nm, 500 nm, or ranges including and/or spanning the aforementioned nm values. In some embodiments, the size distribution of the nanoparticles for at least 90% of the particles present is equal to or less than about: 100 nm, 110 nm, 120 nm, 130 nm, 140 nm, 160 nm, 180 nm, 200 nm, or ranges including and/or spanning the aforementioned nm values. In some embodiments, the D90 of the particles present is equal to or less than about: 80 nm, 100 nm, 110 nm, 120 nm, 130 nm, 140 nm, 160 nm, 180 nm, 200 nm, 300 nm, 400 nm, 500 nm, or ranges including and/or spanning the aforementioned values. In some embodiments, the size of the nanoparticle is the diameter of the nanoparticle as measured using any of the techniques as disclosed elsewhere herein. For instance, in some embodiments, the size of the nanoparticle is the measured using dynamic light scattering. In some embodiments, the size of the nanoparticle is the measured using a zeta sizer.

In some embodiments, the average size of the nanoparticles of a composition as disclosed herein is substantially constant and/or does not change significantly over time (e.g., it is a stable nanoparticle). In some embodiments, after formulation and storage for a period of at least about 1 month (30 days), about 3 months (90 days), or about 6 months (180 days) (e.g., at ambient conditions, at 25° C. with 60% relative humidity, or under the other testing conditions disclosed elsewhere herein), the average size of nanoparticles comprising the composition changes less than or equal to about: 1%, 5%, 10%, 20%, or ranges including and/or spanning the aforementioned values.

In some embodiments, the polydispersity index (PDI) of the nanoparticles of a composition as disclosed herein is less than or equal to about: 0.05, 0.10, 0.15, 0.20, 0.25, 0.30, 0.40, 0.50, 0.60, 0.70, 0.80, or ranges including and/or spanning the aforementioned values. In some embodiments, the size distribution of the nanoparticles is highly monodisperse with a polydispersity index of less than or equal to about: 0.05, 0.10, 0.15, 0.20, 0.25, or ranges including and/or spanning the aforementioned values.

In some embodiments, the zeta potential of the nanoparticles of a composition as disclosed herein is less than or equal to about: 1 mV, 3 mV, 4 mV, 5 mV, 6 mV, 7 mV, 8 mV, 10 mV, 20 mV, or ranges including and/or spanning the aforementioned values. In some embodiments, the zeta potential of the nanoparticles is greater than or equal to about: −3 mV, −1 mV, 0 mV, 1 mV, 3 mV, 4 mV, 5 mV, 6 mV, 7 mV, 8 mV, 4 mV, 10 mV, 20 mV, or ranges including and/or spanning the aforementioned values. In some embodiments, the zeta potential and/or diameter of the particles (e.g., measured using dynamic light scattering) is acquired using a zetasizer (e.g., a Malvern ZS90 or similar instrument).

In some embodiments, the lipid-based particle composition has a pH of less than or equal to about: 2, 3, 4, 5, 6, 6.5, 7, 8, 9, or ranges including and/or spanning the aforementioned values. In some embodiments, the composition has a pH of greater than or equal to about: 2, 3, 4, 5, 6, 6.5, 7, 8, 9, or ranges including and/or spanning the aforementioned values.

In some embodiments, as disclosed elsewhere herein, the lipid-based particle composition is stable. In some embodiments, for example, after formulation (e.g., in water at concentrations disclosed elsewhere herein) and storage for a period of at least about 1 month, 2 months (e.g., equal to or about 90 days), 3 months, or about 6 months, the polydispersity of the nanoparticles changes less than or equal to about: 1%, 5%, 10%, 20%, or ranges including and/or spanning the aforementioned values. In some embodiments, after formulation (e.g., in water at concentrations disclosed elsewhere herein) and storage for a period of at least about 1 month, 2 months (e.g., equal to or about 90 days), 3 months, or about 6 months, the soluble fraction of therapeutic agent (e.g., cannabinoids, such as CBD, non-cannabinoids, and/or combinations of any of the foregoing) in the formulation changes less than or equal to about: 1%, 5%, 10%, 20%, 30%, or ranges including and/or spanning the aforementioned values. In some embodiments, after formulation and storage for a period of at least about 1 month, 2 months (e.g., equal to or about 90 days), or about 6 months (e.g., at ambient conditions, at 25° C. with 60% relative humidity, or under the other testing conditions disclosed elsewhere herein), the PDI of nanoparticles comprising the composition changes by less than or equal to about: 1%, 5%, 10%, 20%, or ranges including and/or spanning the aforementioned values. In some embodiments, after formulation and storage for a period of at least about 1 month, 2 months (e.g., equal to or about 90 days), or about 6 months (e.g., at ambient conditions, at 25° C. with 60% relative humidity, or under the other testing conditions disclosed elsewhere herein), the PDI of nanoparticles comprising the composition changes by less than or equal to about: 0.05, 0.1, 0.2, 0.3, 0.4, or ranges including and/or spanning the aforementioned values. In some embodiments, after formulation and storage for a period of at least about 1 month, 2 months (e.g., equal to or about 90 days), or about 6 months (e.g., at ambient conditions, at 25° C. with 60% relative humidity, or under the other testing conditions disclosed elsewhere herein), the average size of nanoparticles comprising the composition changes by less than or equal to about: 10%, 20%, 30%, 40%, 50%, 100%, or ranges including and/or spanning the aforementioned values. In some embodiments, after formulation and storage for a period of at least about 1 month, 2 months (e.g., equal to or about 90 days), or about 6 months (e.g., at ambient conditions, at 25° C. with 60% relative humidity, or under the other testing conditions disclosed elsewhere herein), the D90 of nanoparticles comprising the composition changes by less than or equal to about: 10%, 20%, 30%, 40%, 50%, 100%, or ranges including and/or spanning the aforementioned values.

In some embodiments, when exposed to simulated gastric fluid (e.g., at a concentration of 20 mg/mL), the particle size of the nanoparticles of a composition as disclosed herein does not change and/or changes less than 5% during a period of greater than or equal to about: 1 hour, 2 hours, 3 hours, 4 hours, 5 hours, 6 hours, 10 hours, or ranges including and/or spanning the aforementioned values. In some embodiments, when exposed to simulated intestinal fluid (e.g., at a concentration of 20 mg/mL), the particle size of the nanoparticles disclosed herein does not change and/or changes less than 5% during a period of greater than or equal to about: 1 hour, 2 hours, 3 hours, 4 hours, 5 hours, 6 hours, 10 hours, or ranges including and/or spanning the aforementioned values. In some embodiments, after formulation (e.g., at a concentration of 20 mg/mL) and storage in simulated gastric fluid for a period of at least about 1 hour or about 2 hours (e.g., at 37° C., or under the other testing conditions disclosed elsewhere herein), the average particle size of nanoparticles comprising the composition changes by less than or equal to about: 1%, 5%, 10%, 20%, 50%, or ranges including and/or spanning the aforementioned values. In some embodiments, after formulation (e.g., at a concentration of 20 mg/mL) and storage in simulated gastric fluid for a period of at least about 1 hour, about 2 hours, about 3 hours, or about 4 hours (e.g., at 37° C. or under the other testing conditions disclosed elsewhere herein), the PDI of nanoparticles comprising the composition changes by less than or equal to about: 1%, 5%, 10%, 20%, or ranges including and/or spanning the aforementioned values. In some embodiments, after formulation (e.g., at a concentration of 20 mg/mL) and storage in simulated gastric fluid for a period of at least about 1 hour or about 2 hours (e.g., at 37° C. or under the other testing conditions disclosed elsewhere herein), the PDI of nanoparticles comprising the composition changes by less than or equal to about: 0.01, 0.05, 0.1, 0.2, 0.3, or ranges including and/or spanning the aforementioned values. In some embodiments, after formulation (e.g., at a concentration of 20 mg/mL) and storage in simulated intestinal fluid for a period of at least about 1 hour or about 2 hours (e.g., at 37° C., or under the other testing conditions disclosed elsewhere herein or under the other testing conditions disclosed elsewhere herein), the average particle size of nanoparticles comprising the composition changes by less than or equal to about: 1%, 5%, 10%, 20%, 50%, or ranges including and/or spanning the aforementioned values. In some embodiments, after formulation (e.g., at a concentration of 20 mg/mL) and storage in simulated intestinal fluid for a period of at least about 1 hour, about 2 hours, about 3 hours, or about 4 hours (e.g., at 37° C. or under the other testing conditions disclosed elsewhere herein), the PDI of nanoparticles comprising the composition changes by less than or equal to about: 1%, 5%, 10%, 20%, 100%, 150%, or ranges including and/or spanning the aforementioned values. In some embodiments, after formulation (e.g., at a concentration of 20 mg/mL) and storage in simulated intestinal fluid for a period of at least about 1 hour, about 2 hours (e.g., at 37° C. or under the other testing conditions disclosed elsewhere herein), the PDI of nanoparticles comprising the composition changes by less than or equal to about: 0.01, 0.05, 0.1, 0.2, 0.3, or ranges including and/or spanning the aforementioned values.

In some embodiments, the composition particle size remains consistent (a size change of less than or equal to about: 0%, 0.5%, 1%, 2%, 3%, 5%, or ranges including and/or spanning the aforementioned values) for a period of at least about 30 days when stored at room temperature, refrigeration, and up to 40° C. In some embodiments, the therapeutic agent (e.g., cannabinoids, such as CBD, non-cannabinoids, and/or combinations of any of the foregoing) concentration in the composition remains consistent (a loss of less than or equal to about: 0.5%, 1%, 2%, 3%, 5%, or ranges including and/or spanning the aforementioned values) for a period of at least about 30 days, 60 days, 90 days, or 120 days when stored at room temperature, refrigeration, and up to 40° C. In some embodiments, when stored at room temperature, refrigeration, and up to 40° C., the composition is stable (e.g., the particle size or therapeutic agent concentration in the nanoparticles remains consistent and/or has a change of less than or equal to about: 0.5%, 1%, 2%, 5%, or ranges including and/or spanning the aforementioned values) for a period of at least about: 2 weeks, 30 days, 2 months, 3 months, 6 months, 9 months, 1 year, or ranges including and/or spanning the aforementioned measures of time.

In some embodiments, the method of using the lipid-based particle composition and/or of treating a subject with the lipid-based particle composition includes administering to a subject in need of treatment (e.g., orally, topically, etc.) an effective amount of the composition. In some embodiments, the composition (e.g., delivery system) improves the stability of CBD (or other cannabinoids, other therapeutics, and/or combinations thereof) after ingestion where the composition is exposed to the stomach and/or intestines in an aqueous environment with harsh pH conditions. In some embodiments, the bioavailability of the therapeutic agent (e.g., cannabinoids, such as CBD, non-cannabinoids, and/or combinations of any of the foregoing) (e.g., in the blood of a subject) relative to the initial administered dose is greater than or equal to about: 10%, 20%, 50%, 75%, or ranges including and/or spanning the aforementioned values. In some embodiments, using the disclosed compositions, the oral bioavailability of the therapeutic agent (e.g., cannabinoids, such as CBD, non-cannabinoids, and/or combinations of any of the foregoing) delivered (as measured using AUC) is higher using an embodiment disclosed herein relative to oral delivery of the therapeutic alone. In some embodiments, the oral bioavailability is improved over the therapeutic alone by greater than or equal to about: 10%, 50%, 75%, 100%, 200%, or ranges including and/or spanning the aforementioned values.

As disclosed elsewhere herein, some embodiments pertain to methods of treating a subject. In some embodiments, the method of treating comprises selecting patient for treatment. In some embodiments, the method of threating comprises administering to the patient an effective amount of a formulation comprising a lipid-based particle composition comprising a therapeutic agent (e.g., cannabinoids, such as CBD, non-cannabinoid therapeutic agents, and combinations thereof).

In some embodiments, compositions as described herein may be used to induce at least one effect, e.g. therapeutic effect, that may be associated with at least one therapeutic agent (e.g., cannabinoids, such as CBD, non-cannabinoids, and/or combinations of any of the foregoing), which is capable of inducing, enhancing, arresting or diminishing at least one effect, by way of treatment or prevention of unwanted conditions or diseases in a subject. As disclosed elsewhere herein, the at least one active agent may be selected amongst therapeutic agents, i.e. agents capable of inducing or modulating a therapeutic effect when administered in a therapeutically effective amount. In some embodiments, the phospholipid, non-phospholipid lipid, sterol, etc. by themselves do not induce or modulate a therapeutic effect but endow the composition (e.g., a pharmaceutical composition) with a selected desired characteristic.

In some embodiments, the compositions disclosed herein (e.g., lipid-based particle compositions including cannabinoids, such as CBD, other therapeutics, and/or combinations thereof) can be used in methods of treatment and can be administered to a subject having a condition to be treated. In some embodiments, the subject is treated by administering an effective amount of a composition (e.g., lipid-based particle compositions including cannabinoids, such as CBD, other therapeutics, and/or combinations thereof) as disclosed herein to the subject.

In some embodiments, the disease or condition to be treated via administration of a composition as disclosed herein may include one or more of pain, anxiety, seizures, malaise, insomnia, inflammation, epilepsy, diabetes, cancer (breast, colon, prostate, glioma, etc.), etc. In some embodiments, the lipid-based particle composition (e.g., those including therapeutic agents such as CBD) is provided for use in treating a condition selected from pain associated disorders (as an analgesic), inflammatory disorders and conditions (as anti-inflammatory), appetite suppression or stimulation (as anoretic or stimulant), symptoms of vomiting and nausea (as antiemetic), intestine and bowl disorders, disorders and conditions associated with anxiety (as anxiolytic), disorders and conditions associated with psychosis (as antipsychotic), disorders and conditions associated with seizures and/or convulsions (as antiepileptic or antispasmodic), sleep disorders and conditions (as anti-insomniac), disorders and conditions which require treatment by immunosuppression, disorders and conditions associated with elevated blood glucose levels (as antidiabetic), disorders and conditions associated with nerve system degradation (as neuroprotectant), inflammatory skin disorders and conditions (such as psoriasis), disorders and conditions associated with artery blockage (as anti-ischemic), disorders and conditions associated with bacterial infections, disorders and conditions associated with fungal infections, proliferative disorders and conditions, disorders and conditions associated with inhibited bone growth, post trauma disorders, and others.

In some embodiments, the lipid-based particle composition (e.g., those comprising cannabinoids, such as CBD, non-cannabinoids, and combinations thereof as disclosed elsewhere herein) is provided for use in a method of treating a subject suffering from a condition selected from pain associated disorders, inflammatory disorders and conditions, symptoms of vomiting and nausea, intestine and bowl disorders, disorders and conditions associated with anxiety, disorders and conditions associated with psychosis, disorders and conditions associated with seizures and/or convulsions, sleep disorders and conditions, disorders and conditions which require treatment by immunosuppression, disorders and conditions associated with elevated blood glucose levels, disorders and conditions associated with nerve system degradation, inflammatory skin disorders and conditions, disorders and conditions associated with artery blockage, disorders and conditions associated with bacterial infections, disorders and conditions associated with fungal infections, proliferative disorders and conditions, and disorders and conditions associated with inhibited bone growth, post trauma disorders and others, a patient in need of appetite suppression or stimulation. In some embodiments, the method comprises administering to the subject an effective amount of a composition of this disclosure.

In some embodiments, the lipid-based particle compositions (e.g., those including cannabinoids, such as CBD, non-cannabinoids, and combinations thereof) described herein may be used for inducing, enhancing, arresting or diminishing at least one effect, by way of treatment or prevention of unwanted conditions or diseases in a subject. The therapeutic agent (substance, molecule, element, compound, entity, or a combination thereof) may be selected amongst therapeutic agents, i.e. agents capable of inducing or modulating a therapeutic effect when administered in a therapeutically effective amount, and non-therapeutic agents, i.e. which by themselves do not induce or modulate a therapeutic effect but which may endow the pharmaceutical composition with a selected desired characteristic.

In some embodiments, a lipid-based particle compositions as disclosed herein (e.g., a pharmaceutical composition comprising a cannabinoid, such as CBD, a non-cannabinoid, and combinations thereof) may be selected to treat, prevent or ameliorate any pathology or condition. In some embodiments, administering of a therapeutic amount of the composition or system described herein, whether in a concentrate form or in a diluted formulation form, is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

Surprisingly and advantageously, several embodiments disclosed herein do not require one or more ingredients typically used to prepare liposomes and/or nanoparticle formulations. In some embodiments, the lipid-based particle compositions disclosed herein lack, contain less than 2%, and/or less than about 0.5% of one or more of lecithin surfactants, hyaluronic acid, Alcolec S, Alcolec BS, Alcolec XTRA-A, polysorbates (such as Polysorbate 80 and Polysorbate 20), monoglycerides, diglycerides, glyceryl oleate, polaxamers, terpenes, sodium alginate, polyvinylpyrrolidone, L-alginate, chondroitin, poly gamma glutamic acid, gelatin, chitosan, corn starch, polyoxyl 40-hydroxy castor oil, Tween 20, Span 80, or the salts of any of thereof. In some embodiments, the lipid-based particle compositions disclosed herein lack, contain less than 2%, and/or less than about 0.5% of a surfactant. In some embodiments, the lipid-based particle compositions disclosed herein (or the active ingredients within these compositions, e.g., cannabinoids) lack, contain less than 3%, contain less than 2%, and/or less than about 0.5% of one or more of THCa, 9-THC, 8-THC, CBDa, CBC, CBG, CBN, THCV, and/or CBGa, individually or collectively. In some embodiments, the CBD lipid-based particle compositions disclosed herein lack, contain less than 2%, and/or less than about 0.5% of one or more of THCa, 9-THC, 8-THC, CBDa, CBC, CBG, CBN, THCV, and/or CBGa. In some embodiments, the lipid-based particle compositions lack unhydrogenated phospholipids. In some embodiments, the lipid-based particle compositions lack hydrogenated phospholipids. In some embodiments, the lipid-based particle compositions comprise one or more unhydrogenated or hydrogenated phospholipids. In some embodiments, the lipid-based particle compositions disclosed herein lack, contain less than 2%, and/or less than about 0.5% of one or more of a buffering agent, a polymeric stabilizing agent, or sodium hydroxide.

In some embodiments, the lipid-based particle compositions disclosed herein lack a nanoparticle structure wherein the structure comprises an outer single layer membrane of essential phospholipids that encapsulates liquid lipids and cannabinoids. As used herein, essential phospholipids are extracts of characteristic fatty acid lipid-based particle composition of the phospholipids distinguished by their particular high content of polyunsaturated fatty acids, predominantly linoleic acid (approx. 70%), linolenic acid and oleic acid and with a high content exceeding 75% of (3-sn-phosphatidyl) choline. Beside phosphatidylcholine molecules, the essential phospholipid fraction includes phosphatidylethanolamine, phosphatidylinositol and other lipids. In some embodiments, the lipid-based particle compositions disclosed herein lack nonnatural ingredients. In some embodiments, the lipid-based particle compositions disclosed are synthetic and not found in nature.

In some embodiments, the lipid-based particle compositions disclosed herein lack, contain less than 2%, and/or less than about 0.5% of one or more organic bases (which may include, but are not limited to: butyl hydroxyl anisole (BHA), butyl hydroxyl toluene (BHT) and sodium ascorbate). In some embodiments, the lipid-based particle compositions disclosed herein lack, contain less than 2%, and/or less than about 0.5% of whey protein isolate. In some embodiments, the lipid-based particle compositions disclosed herein lack, contain less than 2%, and/or less than about 0.5% of ticamulsion 3020, purity gum, gum Arabic, and/or a modified gum Arabic. In some embodiments, the lipid-based particle compositions disclosed herein lack, contain less than 2%, and/or less than about 0.5% one or more of fatty acids, triglycerides triacylglycerols, acylglycerols, fats, waxes, sphingolipids, glycerides, sterides, cerides, glycolipids, sulfolipids, lipoproteins, chylomicrons and the derivatives of these lipids. In some embodiments, the lipid-based particle compositions disclosed herein lack, contain less than 2%, and/or less than about 0.5% of a surfactant. In some embodiments, the lipid-based particle compositions disclosed herein lack, contain less than 2%, and/or less than about 0.5% of one or more of polyglycolized glycerides and polyoxyethylene glycerides of medium to long chain mono-, di-, and triglycerides, such as: almond oil PEG-6 esters, almond oil PEG-60 esters, apricot kernel oil PEG-6 esters (Labrafil® M1944CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-50 esters, hydrogenated castor oil PEG-5 esters, hydrogenated castor oil PEG-7 esters, 9 hydrogenated castor oil PEG-9 esters, corn oil PEG-6 esters (Labrafil® M 2125 CS), corn oil PEG-8 esters (Labrafil® WL 2609 BS), corn glycerides PEG-60 esters, olive oil PEG-6 esters (Labrafil® M1980 CS), hydrogenated palm/palm kernel oil PEG-6 esters (Labrafil® M 2130 BS), hydrogenated palm/palm kernel oil PEG-6 esters with palm kernel oil, PEG-6, palm oil (Labrafil® M 2130 CS), palm kernel oil PEG-40 esters, peanut oil PEG-6 esters (Labrafil® M 1969 CS), glyceryl laurate/PEG-32 laurate (Gelucire® 44/14), glyceryl laurate glyccry I/PEG 20 laurate, glyceryl laurate glyceryl/PEG 32 laurate, glyceryl, laurate glyceryl/PEG 40 laurate, glyceryl oleate/PEG-20 glyceryl, glyceryl oleate/PEG-30 oleate, glyceryl palmitostearate/PEG-32 palmitostearate (Gelucire® 50/13), glyceryl stearate/PEG stearate, glyceryl stearate/PEG-32 stearate (Gelucire® 53/10), saturated polyglycolized glycerides (Gelucire® 37/02 and Gelucire® 50/02), triisostearin PEG-6 esters (i.e. Labrafil® Isostearique), triolein PEG-6 esters, trioleate PEG-25 esters, polyoxyl 35 castor oil (Cremophor® EL or Kolliphor® EL), polyoxyl 40 hydrogenated castor oil (Cremophor® RH 40 or Kolliphor® RH40), polyoxyl 60 hydrogenated castor oil (Cremophor® RH60), polyglycolized derivatives and polyoxyethylene esters or ethers derivatives of medium to long chain fatty acids, propylene glycol esters of medium to long chain fatty acids, which can be used including caprylate/caprate diglycerides, glyceryl monooleate, glyceryl ricinoleate, glyceryl laurate, glyceryl dilaurate, glyceryl dioleate, glyceryl mono/dioleate, polyglyceryl-10 trioleate, poly glyceryl-10 laurate, polyglyceryl-10 oleate, and poly glyceryl-10 mono dioleate, propylene glycol caprylate/caprate (Labrafac® PC), propylene glycol dicaprylate/dicaprate (Miglyol® 840), propylene glycol monolaurate, propylene glycol ricinoleate, propylene glycol monooleate, propylene glycol dicaprylate/dicaprate, propylene glycol dioctanoate, sucrose esters surfactants such as sucrose stearate, sucrose distearate, sucrose palmitate, sucrose oleate, and combinations thereof.

Some embodiments also encompass methods for making (as disclosed elsewhere herein) and for administering the disclosed compositions. Multiple techniques of administering the lipid-based particle compositions as disclosed herein exist including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. In some embodiments, administration is performed through oral pathways, which administration includes administration in an emulsion, capsule, tablet, film, chewing gum, suppository, granule, pellet, spray, syrup, or other such forms. As further examples of such modes of administration and as further disclosure of modes of administration, disclosed herein are various methods for administration of the disclosed compositions including modes of administration through intraocular, intranasal, and intraauricular pathways.

In some embodiments, where a topical is provided, topical permeation enhancers may be included and may be selected from, but not inclusive of, the following: dimethyl sulfoxide, dimethyl sulfone, ethanol, propylene glycol, dimethyl isosorbide, polyvinyl alcohol, Capryol™ 90, Labrafil M1944 CS, Labrasol, Labrasol ALF, LauroglycolT M90, Transcutol HP, Capmul S 12L, Campul PG-23 EP/NF, Campul PG-8 NF. The topical may include one or more of Lipoid's Skin Lipid Matrix 2026 technology, lipid/oil based ingredients or oil soluble ingredients, and includes Captex 170 EP as a skin permeation enhancer, argan oil, menthol, arnica oil, camphor, grapefruit seed oil, For example, dimethyl sulfoxide, dimethyl isosorbide, topical analgesics such as lidocaine, wintergreen oil, and terpenes such as guaiacol. In some embodiments, any one or more of these ingredients is present in the topical composition at a dry wt % of equal to or less than about: 5%, 10%, 15%, 20%, 30%, 40%, 50%, 60%, or ranges including and/or spanning the aforementioned values. In some embodiments, any one or more of these ingredients is present in the topical at a wet wt % of equal to or at least about: 2.5%, 5%, 7.5%, 10%, 12.5%, 15%, 20%, 30%, or ranges including and/or spanning the aforementioned values.

In some embodiments, the lipid-based particle compositions disclosed herein can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). In some embodiments, these additional agents are not added. Such preparations can include liposomes, microemulsions, micelles, and/or unilamellar or multilamellar vesicles.

For oral administration, the lipid-based particle compositions can be provided as a tablet, aqueous or oil suspension, dispersible powder or granule (as a food additive, drink additive, etc.), emulsion, hard or soft capsule, syrup or elixir. Compositions intended for oral use can include one or more of the following agents: sweeteners, flavoring agents, coloring agents and preservatives. In some embodiments, the compositions are provided in ready-to-drink formulations, such as protein drinks, energy drinks, sodas, juices, coffees, etc.

In some embodiments, as mentioned elsewhere herein, the compositions disclosed herein, when provided in a ready-to-drink beverage, are stable during ozonation sterilization, UV sterilization, heat sterilization (e.g., pasteurization), filtration sterilization, and/or gamma irradiation during beverage preparation and packaging. In some embodiments, the particle size and/or PDI after sterilization (e.g., exposure to techniques that allow sterilization of the composition) varies by less than or equal to about: 1%, 5%, 10%, 20%, 30%, 40%, 50%, 60%, or ranges including and/or spanning the aforementioned values. In some embodiments, the therapeutic agent (e.g., cannabinoids, such as CBD, non-cannabinoids, and/or combinations of any of the foregoing) concentration after sterilization (e.g., exposure to techniques that allow sterilization of the composition) drops by less than or equal to about: 0.05%, 0.1%, 0.5%, 1%, 5%, 10%, 15%, or ranges including and/or spanning the aforementioned values. In some embodiments, after stabilization, the beverages comprising lipid-based particle compositions have a shelf life of equal to or greater than 6 months, 12 months, 14 months, 16 months, 18 months, 19 months, or ranges including and/or spanning the aforementioned values (e.g., a standard storage conditions).

In some embodiments, where sterilization is performed using pasteurization, the pasteurization may be performed at a final therapeutic (e.g., CBD) concentration of 0.1 mg/mL in water. In several embodiments, the pasteurization is carried out using a pasteurization time for a period of equal to or at least about: 1 second, 15 seconds, 30 seconds, 1 minute, 5 minutes, 10 minutes, 20 minutes, 30 minutes, 1 hour, or ranges including and/or spanning the aforementioned values. In several embodiments, the pasteurization is carried out using a pasteurization temperature of equal to or at least about: 89° C., 72° C., 63° C., 50° C., or ranges including and/or spanning the aforementioned values. In some embodiments, the particle size and/or PDI after pasteurization (e.g., measured directly after sterilization) varies by less than or equal to about: 0.01%, 0.05%, 0.1%, 0.5%, 1%, 2.5%, 5%, or ranges including and/or spanning the aforementioned values. In some embodiments, the change in particle size and/or PDI after pasteurization relative to a non-sterilized control (e.g., measured one week after sterilization) varies by less than or equal to about: 0.01%, 0.05%, 0.1%, 0.5%, 1%, 2.5%, 5%, 8%, 10%, or ranges including and/or spanning the aforementioned values. In several embodiments, the particle size and/or PDI measurement is performed directly after (e.g., substantially immediately) sterilization and/or a week or more after sterilization (e.g., 1 week, two weeks, etc.).

In some embodiments, where sterilization is performed using ozonation, the ozonation may be performed at a final therapeutic (e.g., CBD) concentration of 0.2 mg/mL in water. In several embodiments, the ozonation is carried out using a ozonation time (e.g., using a commercial ozone air and water purifier) for a period of equal to or at least about: 1 minute, 5 minutes, 10 minutes, 20 minutes, 30 minutes, 1 hour, or ranges including and/or spanning the aforementioned values. In some embodiments, the particle size and/or PDI after ozonation sterilization (e.g., measured directly after sterilization) varies by less than or equal to about: 0.01%, 0.05%, 0.1%, 0.5%, 1%, 2.5%, 5%, or ranges including and/or spanning the aforementioned values. In some embodiments, the change in particle size and/or PDI after ozonation relative to a non-sterilized control (e.g., measured one week after sterilization) varies by less than or equal to about: 0.01%, 0.05%, 0.1%, 0.5%, 1%, 2.5%, 5%, 8%, 10%, or ranges including and/or spanning the aforementioned values. In several embodiments, the particle size and/or PDI measurement is performed directly after (e.g., substantially immediately) sterilization and/or a week or more after sterilization (e.g., 1 week, two weeks, etc.).

In some embodiments, where sterilization is performed using UV treatment, the UV treatment may be performed at a final therapeutic (e.g., CBD) concentration of 0.2 mg/mL in water. In several embodiments, the UV treatment is carried out using at least 1, 2, 5, 10, UV cycles, or ranges including and/or spanning the aforementioned values. In some embodiments, the particle size and/or PDI after UV treatment (e.g., measured directly after sterilization) varies by less than or equal to about: 0.01%, 0.05%, 0.1%, 0.5%, 1%, 2.5%, 5%, or ranges including and/or spanning the aforementioned values. In some embodiments, the change in particle size and/or PDI after UV treatment relative to a non-sterilized control (e.g., measured one week after sterilization) varies by less than or equal to about: 0.01%, 0.05%, 0.1%, 0.5%, 1%, 2.5%, 5%, 8%, 10%, or ranges including and/or spanning the aforementioned values. In several embodiments, the UV treatment may be performed using UVA, UVB, UVC, full spectrum UV light, and/or combinations of the foregoing. In several embodiments, the particle size and/or PDI measurement is performed directly after (e.g., substantially immediately) sterilization and/or a week or more after sterilization (e.g., 1 week, two weeks, etc.).

The shelf-life can be determined as the period of time in which there is 95% confidence that at least 50% of the response (therapeutic agent(s) concentration or particle size) is within a specification limit. A specification limit is a range of measured values in which a quality parameter should be within in order for products to be considered of the same quality when it was initially released. For example, where the CBD target concentration is 20 mg/mL, a specification limit may be defined as 18 to 22 mg/mL. At a time during a stability study, the CBD concentration may fall below 18 mg/mL due to chemical instability, at that time the product may be considered out of specification.

In some embodiments, the shelf life determined as a time where the concentration of the active ingredient has changed (e.g., lessened) by less than or equal to 15%, 10%, 5%, 2.5%, or ranges including and or spanning the aforementioned ranges.

In some embodiments, the sterilized beverage may be a cold beverage (e.g., juices, sports drinks, energy drinks, protein drinks, nutritional drinks, sodas, etc.). In some embodiments, the cold beverage may be a carbonated beverage. In some embodiments, the cold beverage may be an alcoholic beverage.

In some embodiments, the compositions may be provided in hot beverages (e.g., coffee, tea, etc.). In some embodiments, after a 30 minute period in a hot beverage, the particle size and/or PDI varies by less than or equal to about: 1%, 5%, 10%, 20%, 30%, or ranges including and/or spanning the aforementioned values. In some embodiments, after a 30 minute period in a hot beverage, the therapeutic agent (e.g., cannabinoids, such as CBD, non-cannabinoids, and/or combinations of any of the foregoing) concentration drops by less than or equal to about: 1%, 5%, 10%, 15%, or ranges including and/or spanning the aforementioned values.

In some embodiments, surprisingly, an aqueous lipid-based particle composition comprising a therapeutic agent (e.g., cannabinoids, such as CBD, non-cannabinoids, and combinations thereof) as disclosed herein may be administered using an atomizer. In some embodiments, an atomizer nozzles are used in oral spray, such as the binaca spray. In some embodiments, an atomizer nozzle is used in a nasal spray. This result is surprising, as cannabinoid formulations would be typically be understood to clog atomizer nozzles.

Formulations for oral use can also be provided as gelatin capsules. In some embodiments, a powder composition as disclosed herein is added to the gelatin capsule. In some embodiments, the active ingredient(s) in the nanoparticle compositions disclosed herein are mixed with an inert solid diluent, such as calcium carbonate, calcium phosphate, or kaolin, or as soft gelatin capsules. In soft capsules, the active compounds can be dissolved or suspended in suitable liquids, such as water. Stabilizers and microspheres formulated for oral administration can also be used. Capsules can include push-fit capsules made of gelatin, as well as soft, sealed capsules made of gelatin and a plasticizer, such as glycerol or sorbitol.

In capsule formulations, trehalose can be added. In some embodiments, trehalose is present in the lipid-based particle composition at a dry wt % of equal to or less than about: 5%, 10%, 15%, 20%, 30%, 40%, 50%, 60%, or ranges including and/or spanning the aforementioned values. In some embodiments, the trehalose is present in the composition at a wet wt % of equal to or at least about: 2.5%, 5%, 7.5%, 10%, 12.5%, 15%, 20%, 30%, or ranges including and/or spanning the aforementioned values.

As noted elsewhere herein, in some embodiments, the lipid-based particle composition lacks terpenes (e.g., as impurities or additives). However, in other embodiments, one or more terpenes may be added to prepare the nanoparticle composition. In some embodiments, the one or more terpenes includes one or more of alpha fenchone, alpha terpinene, alpha terpineol, beta caryophyllene, alpha pinene, beta pinene, bisabolene, bisabolol, borneol, eucalyptol, gamma terpinene, guaiacol, humulene, linalool, myrcene, para cymene, phytol, and/or terpinolene. In some embodiments, the one or more terpenes includes one or more of 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, B eta-Sinensal, B eta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, Vanillin, or any terpene provided in any of the Examples below.

In some embodiments, the one or more terpenes, collectively or individually, are present in the aqueous composition at a concentration of less than or equal to about: 400 mg/ml, 300 mg/ml, 200 mg/ml, 150 mg/ml, 100 mg/ml, 75 mg/ml, 50 mg/ml, 25 mg/ml, or ranges including and/or spanning the aforementioned values. In some embodiments, the one or more terpenes (collectively or individually) are present in the composition at a dry wt % of equal to or less than about: 5%, 10%, 15%, 20%, 30%, 40%, 50%, 60%, or ranges including and/or spanning the aforementioned values. In some embodiments, the one or more terpenes (collectively or individually) are present in the composition at a wet wt % of equal to or less than about: 2.5%, 5%, 7.5%, 10%, 12.5%, 15%, 20%, 30%, 40%, or ranges including and/or spanning the aforementioned values.

Dry powder formulations or liquid embodiments may also be used in a variety of consumer products. For example, in some embodiments, dry powders can be added (e.g., scooped, from a packet, squirted from a dispenser, etc.) into any consumer product. In some embodiments, liquid formulations can be added measured and poured into any consumer product. In some embodiments, the consumer product can include one or more alcoholic beverages, milks (dairy, but also nuts “milks” such as almond juice, etc.), coffee, sodas, tea, fermented beverages, wines, nutritional supplements, smoothies, simple water, sports drinks, sparkling water, or the like. In some embodiments, the consumer product can include one or more eye drops, mouth wash, lotions/creams/serums, lip balms, hair care products, deodorant, nasal solutions, enema solutions, liquid soaps, solid soaps, or the like. In some embodiments, the consumer product can include one or more food products. In some embodiments, the consumer product can include desserts. In some embodiments, the consumer product can include single serving products of multi-serving products (e.g., family size). In some embodiments, the consumer product can include one or more dried products (e.g., flour, coffee creamer, protein shakes, nutritional supplements, etc.). In some embodiments, these dried products can be configured to be reconstituted for use. In some embodiments, the consumer product can include one or more the dried product can be added to other dietary supplements (e.g., multivitamins, gummies, etc.).

Several illustrative embodiments of compositions and methods have been disclosed. Although this disclosure has been described in terms of certain illustrative embodiments and uses, other embodiments and other uses, including embodiments and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various embodiments. All possible combinations and subcombinations of elements and components described herein are intended to be included in this disclosure. No single feature or group of features is necessary or indispensable.

Certain features that are described in this disclosure in the context of separate implementations can also be implemented in combination in a single implementation. Conversely, various features that are described in the context of a single implementation also can be implemented in multiple implementations separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, structures, and/or devices disclosed or illustrated in one embodiment, flowchart, or example in this disclosure can be combined or used with (or instead of) any other portion of any of the steps, processes, structures, and/or devices disclosed or illustrated in a different embodiment, flowchart, or example. The embodiments and examples described herein are not intended to be discrete and separate from each other. Combinations, variations, and other implementations of the disclosed features are within the scope of this disclosure.

The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic.

Conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Additionally, the phrase “consisting essentially of” will be understood to include those elements specifically recited and those additional elements that do not materially affect the basic and novel characteristics of the claimed technology. The phrase “consisting of” excludes any element not specified.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Further, while illustrative embodiments have been described, any embodiments having equivalent elements, modifications, omissions, and/or combinations are also within the scope of this disclosure. Moreover, although certain aspects, advantages, and novel features are described herein, not necessarily all such advantages may be achieved in accordance with any particular embodiment. For example, some embodiments within the scope of this disclosure achieve one advantage, or a group of advantages, as taught herein without necessarily achieving other advantages taught or suggested herein. Further, some embodiments may achieve different advantages than those taught or suggested herein.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise.

### Enumerated Embodiments

The following are provided for the illustration of certain embodiments of the invention.

1. A lipid-based particle composition, comprising:
a nanoparticle comprising:
a first therapeutic agent;
a phospholipid comprising;
cholesterol; and
a medium chain triglyceride; and
water.

2. The lipid-based particle composition of embodiment 1, wherein the lipid-based particle composition is in the form of liposomes and/or an oil-in-water nano-emulsion; and/or wherein the nanoparticles have an average size ranging from about 75 nm to about 500 nm; and/or wherein, upon storage for a period of one month, the average size of the nanoparticles changes by less than about 20%; and/or wherein, when exposed to simulated gastric fluid at a pH of 1.6 for a period of at least 1 hour, the average size of the nanoparticles changes less than or equal to 10%; and/or wherein, when exposed to simulated intestinal fluid at a pH of 6.5 for a period of at least 1 hour, the average size of the nanoparticles changes less than or equal to 10%; and/or wherein, upon exposure to sterilization conditions, the average size of the nanoparticles changes less than 2%.

3. The lipid-based particle composition of embodiment 1 or 2, wherein an appreciable amount of the nanoparticle lipid-based particle composition does not settle and/or separate from the water upon standing for a period of at least about 12 hours.

4. The lipid-based particle composition of any one of embodiments 1 to 3, wherein the lipid-based particle composition is configured such that when concentrated to dryness to afford a powder formulation of nanoparticles, the nanoparticle powder can be reconstituted to provide the nanoparticle lipid-based particle composition.

5. The lipid-based particle composition of any one of embodiments 1 to 4, wherein the first therapeutic agent is present in an amount of less than or equal to about 25 mg/ml.

6. The lipid-based particle composition of any one of embodiments 1 to 5, wherein the phosphatidylcholine is present in an amount of less than or equal to about 100 mg/ml.

7. The lipid-based particle composition of any one of embodiments 1 to 6, wherein the cholesterol is present in an amount of less than or equal to about 25 mg/ml.

8. The lipid-based particle composition of any one of embodiments 1 to 7, wherein the lipid is present in an amount of less than or equal to about 100 mg/ml.

9. The lipid-based particle composition of any one of embodiments 1 to 8, wherein the lipid comprises hemp oil.

10. The lipid-based particle composition of any one of embodiments 1 to 9, further comprising a preservative.

11. The lipid-based particle composition of embodiment 10, wherein the preservative comprises one or more of malic acid, citric acid, potassium sorbate, sodium benzoate, and Vitamin E.

12. The lipid-based particle composition of embodiment 11, wherein the malic acid is present in an amount of less than or equal to about 0.85 mg/ml.

13. The lipid-based particle composition of embodiment 11, wherein the citric acid is present in an amount of less than or equal to about 0.85 mg/ml.

14. The lipid-based particle composition of embodiment 11, wherein the potassium sorbate is present in an amount of less than or equal to about 1 mg/ml.

15. The lipid-based particle composition of embodiment 11, wherein the sodium benzoate is present in an amount of less than or equal to about 1 mg/ml.

16. The lipid-based particle composition of any one of embodiment 1 to 15, further comprising a flavoring agent.

17. A nanoparticle lipid-based particle composition, comprising:
a nanoparticle comprising:
a phospholipid;
a triglyceride;
a sterol; and
a first therapeutic agent;
water;
wherein any one or more of the following (or none) applies: an appreciable amount of the nanoparticle lipid-based particle composition does not settle and/or separate from the water upon standing for a period of at least about 12 hours; the lipid-based particle composition is in the form of liposomes and/or an oil-in-water nano-emulsion; the nanoparticles have an average size ranging from about 75 nm to about 500 nm; upon storage for a period of one month, the average size of the nanoparticles changes by less than about 20%; when exposed to simulated gastric fluid at a pH of 1.6 for a period of at least 1 hour, the average size of the nanoparticles changes less than or equal to 10%; when exposed to simulated intestinal fluid at a pH of 6.5 for a period of at least 1 hour, the average size of the nanoparticles changes less than or equal to 10%; and/or wherein, upon exposure to sterilization conditions, the average size of the nanoparticles changes less than 2%.

18. The lipid-based particle composition of embodiment 17, wherein the phospholipid is selected from the group consisting of phosphatidic acid, phosphatidylethanolamine, phosphatidylcholine, phosphatidylserine, phosphatidylinositol, phosphatidylinositol phosphate, phosphatidylinositol bisphosphate, and phosphatidylinositol trisphosphate.

19. The lipid-based particle composition of embodiment 17 or 18, wherein the triglyceride is a medium chain triglyceride.

20. The lipid-based particle composition of embodiment 19, wherein the medium chain triglyceride comprises one or more of caprioc acid, octanoic acid, capric acid, and/or lauric acid.

21. The lipid-based particle composition of any one of embodiments 17 to 20, wherein the sterol is cholesterol.

22. The lipid-based particle composition of any one of embodiments 1 to 21, wherein the wherein the first therapeutic agent comprises a cannabinoid or a non-cannabinoid.

23. The lipid-based particle composition of any one of embodiments 1 to 22, further comprising a second therapeutic agent.

24. The lipid-based particle composition of embodiment 23, wherein the second therapeutic agent is a cannabinoid or a non-cannabinoid.

25. The lipid-based particle composition of embodiment 23, wherein the first therapeutic agent is CBD and the second therapeutic agent is a cannabinoid that is not CBD.

26. The lipid-based particle composition of embodiment 23, wherein the first therapeutic agent is CBD and the second therapeutic agent is a non-cannabinoid.

27. The lipid-based particle composition of any one of embodiments 23 to 26, further comprising additional therapeutic agents

28. A method of treating a patient in need of treatment comprising administering an effective amount of the lipid-based particle composition of any one of embodiments 1 to 27 to the patient.

29. A method of manufacturing a nanoparticle lipid-based particle composition of a therapeutic agent, comprising:
mixing the therapeutic agent and one or more phospholipids to provide a solution; and
passing the solution through a microfluidizer.

30. The method of embodiment 29, further comprising adding one or more sterols to the solution.

31. The method of embodiment 29 or 30, further comprising adding one or more lipids to the solution.

32. A method of manufacturing a nanoparticle lipid-based particle composition of a therapeutic agent, comprising:
mixing the therapeutic agent and one or more phospholipids to provide a solution;
drying the solution to provide a substantially solid product;
constituting the product in water to provide a reconstituted solution; and
passing the reconstituted solution through a microfluidizer.

33. The method of embodiment 32, further comprising adding one or more sterols to the solution.

34. The method of embodiment 32 or 33, further comprising adding one or more lipids to the solution.

35. A lipid-based particle lipid-based particle composition, comprising:
a nanoparticle comprising:
a therapeutic agent that is of sufficient purity that it exists in a solid and/or powdered state prior to formulation in the nanoparticle lipid-based particle composition at a weight percent in the lipid-based particle composition ranging from 1% to 10%;
a phosphatidylcholine at a weight percent in the lipid-based particle composition ranging from 2.5% to 15%;
a sterol at a weight percent in the lipid-based particle composition ranging from 0.5% to 5%; and
a medium chain triglyceride at a weight percent in the lipid-based particle composition ranging from 2.5% to 15%; and
water at a weight percent in the lipid-based particle composition ranging from 60% to about 80%;
wherein the nanoparticles have an average size ranging from about 75 nm to about 175 nm; and
wherein, upon storage for a period of one month, the average size of the nanoparticles changes by less than about 20%.

36. The lipid-based particle lipid-based particle composition of embodiment 35, wherein the lipid-based particle composition is in the form of liposomes and/or an oil-in-water nano-emulsion.

37. The lipid-based particle lipid-based particle composition of embodiment 35 or 36, wherein an appreciable amount of the nanoparticle lipid-based particle composition does not settle and/or separate from the water upon standing for a period of at least about 12 hours.

38. The lipid-based particle lipid-based particle composition of any one of embodiments 35 to 37, wherein the lipid-based particle composition is configured such that when concentrated to dryness to afford a powder formulation of nanoparticles, the nanoparticle powder can be reconstituted to provide the nanoparticle lipid-based particle composition.

39. The lipid-based particle lipid-based particle composition of any one of embodiments 35 to 38, wherein the lipid-based particle composition has a Tmax for CBD of less than 4.5 hours.

40. The lipid-based particle lipid-based particle composition of any one of embodiments 35 to 39, wherein, upon storage for a period of one month, the average size of the nanoparticles changes by less than about 20%.

41. The lipid-based particle lipid-based particle composition of any one of embodiments 35 to 40, wherein the polydispersity of the nanoparticles in the lipid-based particle composition is less than or equal to 0.15.

42. The lipid-based particle lipid-based particle composition of any one of embodiments 35 to 41, wherein upon 90 days of storage at 25° C. and 60% relative humidity, the polydispersity of the nanoparticles changes by less than or equal to 10%.

43. The lipid-based particle lipid-based particle composition of any one of embodiments 35 to 42, wherein upon 90 days of storage at 25° C. and 60% relative humidity, the polydispersity of the nanoparticles changes by less than or equal to 0.1.

44. The lipid-based particle lipid-based particle composition of any one of embodiments 35 to 43, wherein lipid-based particle composition has a shelf life of greater than 18 months at 25° C. and 60% relative humidity.

45. The lipid-based particle lipid-based particle composition of any one of embodiments 35 to 44, upon 90 days of storage at 25° C. and 60% relative humidity, the D90 of the nanoparticles changes less than or equal to 10%, 20%, 30%, 40%, or 50%.

46. The lipid-based particle lipid-based particle composition of any one of embodiments 35 to 45, wherein the lipid-based particle composition has a concentration max (Cmax) of 80 ng/ml after an oral dose of 15 mg/kg.

47. A lipid-based particle lipid-based particle composition, comprising:
a nanoparticle comprising:
cannabidiol (CBD) that is of sufficient purity that it exists in a solid and/or powdered state prior to formulation in the nanoparticle lipid-based particle composition at a weight percent in the lipid-based particle composition ranging from 5% to 15%;
a phosphatidylcholine at a weight percent in the lipid-based particle composition ranging from 35% to 60%;
a sterol at a weight percent in the lipid-based particle composition ranging from 2.5% to 10%; and
a medium chain triglyceride at a weight percent in the lipid-based particle composition ranging from 35% to 50%;
wherein the lipid-based particle composition has a Cmax of 80 ng/ml after an oral dose of 15 mg/kg.

48. The lipid-based particle lipid-based particle composition of embodiment 47, wherein the lipid-based particle lipid-based particle composition is provided as a dry powder.

49. The lipid-based particle of embodiment 48, wherein the powder is configured to be reconstituted in water to provide an aqueous solution.

50. The lipid-based particle of embodiment 48 or 49, wherein, upon reconstitution, nanoparticles within the aqueous solution have an average size ranging from about 75 nm to about 175 nm.

51. The lipid-based particle lipid-based particle composition of any one of embodiments 35 to 50, further comprising a preservative.

52. The lipid-based particle lipid-based particle composition of embodiment 51, wherein the preservative comprises one or more of malic acid, citric acid, potassium sorbate, sodium benzoate, and Vitamin E.

53. The lipid-based particle lipid-based particle composition of any one of embodiments 35 to 52, wherein the sterol is cholesterol.

54. The lipid-based particle lipid-based particle composition of any one of embodiment 35 to 53, further comprising a flavoring agent.

55. A method of treating a patient in need of treatment comprising administering an effective amount of the lipid-based particle lipid-based particle composition of any one of embodiments 35 to 54 to the patient.

56. A method of manufacturing a nanoparticle lipid-based particle composition of a therapeutic agent, comprising:
providing the therapeutic agent;
providing phosphatidylcholine;
providing a medium chain triglyceride;
mixing the medium chain triglyceride, phosphatidylcholine, and therapeutic agent to provide a solution; and
passing the solution through a microfluidizer to provide a lipid-based particle lipid-based particle composition.

57. The method of embodiment 56, further comprising adding one or more sterols to the solution.

58. The method of embodiment 56 to 57, further comprising adding water to the solution.

### Examples

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art. Formulations were prepared using ingredient profiles and techniques as disclosed herein. The impact of several quality attributes of the formulation on particle size, CBD concentration, and product stability were determined. Such product attributes included CBD to lipid ratio and the preservative system and overall impact of pH. The dissolution and stability of the product was measured in simulated gastric and intestinal fluids. Additionally, the oral pharmacokinetics of embodiments disclosed herein were measured in a mini-pig model and compared to two oil-based commercial products. The physical and chemical stability of embodiments disclosed herein were determined under several storage conditions.

### Example 1: Preparation of an Embodiment of the Composition

### Materials and Methods

Unless otherwise noted, the ingredients used herein were obtained from the following vendors: Sunflower derived phosphatidylcholine and medium chain triglycerides were purchased from American Lecithin Company (a Lipoid Company listed as “MCT”), potassium sorbate, peppermint oil, vitamin E, malic acid, and cholesterol were purchased from Spectrum Chemicals, CBD isolate was purchased from Botanical & Bioscience Laboratories, Luo Han Guo (monk fruit) extract was purchased from GLG Life Tech Corporation, water for injection was purchased from Rocky Mountain Biologicals, and citric acid monohydrate and sodium benzoate were purchased from JT Baker. The CBD isolate used comprised not more than 0.3% THC by weight per weight (w/w). The phosphatidylcholine was H 100-3 grade including over 96.3% or 99.9% phosphatidylcholine (hydrogenated). The phosphatidyl choline and less than 1.1% lysophosphatidylcholine and less than 2.0% triglycerides. This is a highly purity phosphatidylcholine (over 96% pure phosphatidylcholine (hydrogenated)) which is, to the inventor's knowledge, not used in current CBD products.

Particle size and zeta potential of liquid was measured on a Malvern ZS90 Zetasizer (Malvern, UK). The liquid product was diluted at least 50 times in purified water and the equivalent of 1 mg of CBD in a powder form was dissolved in 1 mL of purified water for measurements. Products were measured in low-volume, disposable cuvettes and zeta cassettes. Cannabinoids and terpenes concentrations, related substances and identity (retention time) were measure by high-pressure liquid chromatography (HPLC) at 374 Labs (Reno, Nev.). Residual solvents and pesticides were measured by gas chromatography (GC), and heavy metals by inductive coupled plasma-optical emission spectrometry (ICP-oES) at 374 Labs. Rapid preservative effectiveness testing was determined by a reduction in colony forming units (CFU) of test microorganisms at Microchem Laboratory (Round Rock, Tex.). Testing confirmed that the compositions were resistant to bacterial growth (by measuring colony forming units (CFUs) per volume in a given amount of time.

Manufacturing Process: CBD lipid nanoparticles in this example were prepared using a solvent-based method with high pressure homogenization. To prepare the nanoparticle composition lipophilic ingredients (solid CBD comprising not more than 0.3% THC, medium chain triglyceride, cholesterol, phosphatidylcholine, Vitamin E, oil soluble flavoring, etc.) were accurately weighed onto a weigh boat and then transferred to a 20 liter, glass, round-bottom flask. To the lipophilic ingredients, approximately 1.3 to 1.5 times the weight of the lipophilic ingredients was added of 100% (200 proof) ethanol. The lipophilic ingredients were dissolved in the ethanol before proceeding. The 20 liter round bottom flask was transferred to a Hei-VAP Industrial Rotary Evaporator (Heidolph Corporation) and the ethanol was removed by evaporation under reduced pressure, elevated temperature, and vessel rotation. When the ethanol was removed, a film of lipid remained on the glass vessel walls. The lipid film was blanketed with nitrogen glass and left at room temperature overnight.

All water-soluble formulation ingredients (water soluble flavoring, sodium benzoate, potassium sorbate, citric acid monohydrate, malic acid, etc.) were dissolved into water for injection at the specified concentrations (below). Aqueous solutions were heated and filtered prior to further use. An appropriate amount of aqueous solution was transferred to the glass vessel containing the dried lipid ingredients. The glass vessel was transferred to a heating mantel and warmed with constant stirring from an overhead mixer. Mixing was continued until a homogenous slurry of lipids in water was formed. The full volume of lipid slurry was processed through a microfluidizer (Microfluidics Corporation) 0 to 10 times at a processing pressure of 10,000-30,000 PSI. Alternatively, the volume of lipid slurry can be processed at a pressure of 10,000-30,000 PSI such that the material is recirculated back into the unprocessed volume for a period of time until the desired particle size characteristics are achieved. The resulting lipid nanoparticle solution was cooled with continuous stirring for 12-24 hours before characterizing and fill-finish. Flavoring in oil form was introduced into the dried lipid film prior to introduction of the aqueous solution. Water soluble flavoring is dissolved into the water for injection prior to introduction into the lipid film.

Four batches of approximately 10 liters of CBD isolate containing lipid nanoparticles each were prepared in a cGMP facility according to the Manufacturing Process described above. The ingredient composition of each batch is described in the table below.

TABLE 1

| Ingredient | Batches 1 and 3 | | Batches 2 and 4 | |
|---|---|---|---|---|
| Oil Soluble Flavoring | 0.12% | (w/w) | 0.00% | (w/w) |
| Vitamin E Oil | 0.05% | (w/w) | 0.05% | (w/w) |
| Sodium Benzoate | 0.10% | (w/w) | 0.10% | (w/w) |
| Potassium Sorbate | 0.10% | (w/w) | 0.10% | (w/w) |
| Citric Acid Monohydrate | 0.10% | (w/w) | 0.10% | (w/w) |
| Malic Acid | 0.01% | (w/w) | 0.01% | (w/w) |
| Water Soluble Flavoring | 0.09% | (w/w) | 0.09% | (w/w) |
| Sunflower | 10.08% | (w/w) | 10.08% | (w/w) |
| Phosphatidylcholine | | | | |
| Medium Chain Triglyceride | 9.67% | (v/w) | 9.67% | (v/w) |
| CBD Isolate | 2.01% | (w/w) | 2.01% | (w/w) |
| Cholesterol | 1.01% | (w/w) | 1.01% | (w/w) |
| Ethanol | <0.10% | (w/w) | <0.10% | (w/w) |
| Water for Injection | 76.65% | (v/w) | 76.79% | (v/w) |

### Example 2: Stability Testing

This example discloses stability testing and shelf-life data for some embodiments as prepared in Example 1. Upon cooling, the batches prepared in Example 1 were filled into 20 mL amber vials with a child-proof cap affixed with a required removal torque of 7.0 to 9.0 pound force inch. Sealed bottles were stored at 2-8° C., 25° C./60% Relative Humidity, 40° C./75% relative humidity, or 50° C. and uncontrolled humidity. At a minimum, samples were pulled for characterization on months 0, 1, 2, 3, 6, and 11. Characterization included particle size analysis by dynamic light scattering and CBD concentration by UPLC. Results are show in FIGS. 3 and 4.

Shelf-life plots were created in MiniTab Version 17.0 using real-time data only (25° C./60% Relative Humidity). The shelf-life is the period of time in which there is 95% confidence that at least 50% of the response (CBD concentration or particle size) is within the specification limit. Shown in FIG. 3 is the shelf-life plot of 4 batches of product as a function of CBD concentration. Over the 11 months where CBD concentration was determined, the response slope of the regression line is not significantly different from zero and no shelf-life can be predicted until a negative slope (ie degradation) appears in the data set. FIG. 4 shows the shelf-life plot of 4 batches of product as a function of lipid nanoparticle Z-Average size in nanometers. An upper specification limit of 200 nm was chosen and shelf-life of 565.5 days or approximately 19 months is estimated. Taken together, formulation quality attributes of CBD concentration and particle size remain within the product specification for an estimated 19 months, indicating the product has a shelf-life of 19 months.

### Example 3: Imaging of Nanoparticles

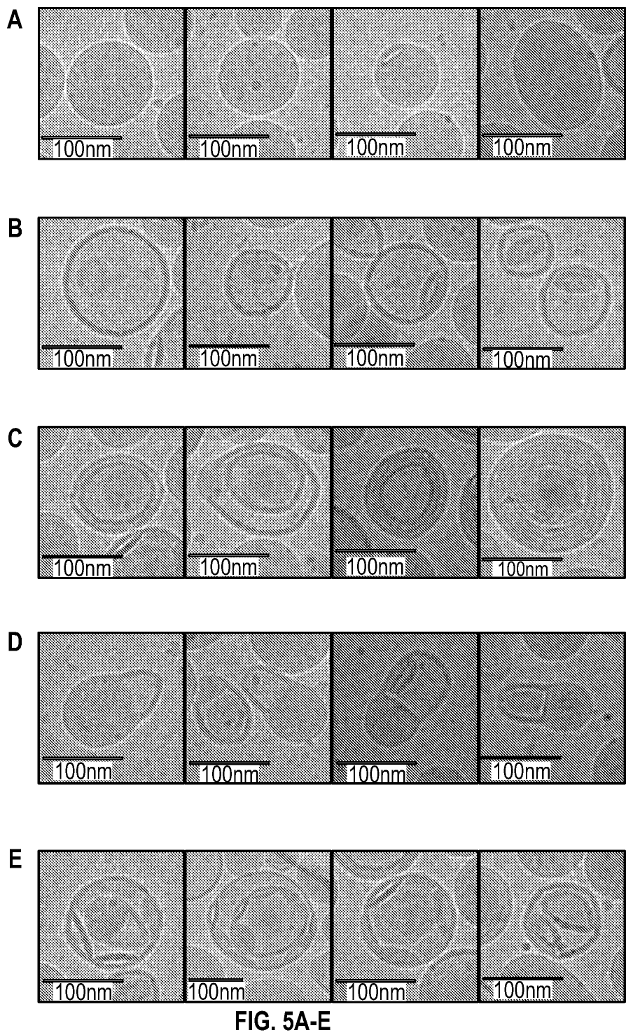

This example discloses representative images of lipid nanoparticles prepared as previously described in Example 1. A sample consistent with the ingredient composition outlined in Batch 1 and 3 was diluted 10 times with water. Three microliters was placed on a thin copper grid (Cu-200CN, Pacific Grid-Tech) that was previously glow-discharged. For preparation of the grid, the sample was loaded into the freezing chamber at low temperature (0-5° C.) under humidity control (100%). After blotting for 2 seconds with filter paper, the specimen was rapidly frozen with cryogen, liquid ethane cooled by liquid nitrogen. The prepared dried was mounted on 200 kV FEI Talos C200C electron microscope. Microscope images were collected at 45K magnification. Example images are shown in FIG. 5.

Lipid nanoparticles prepared using the methods of Example 1 afforded several sub-types of particles. Shown in FIG. 5 Panel A are characteristic emulsion style particles, FIG. 5 Panel B shows lipid nanoparticles containing unilamellar vesicles, also known as small unilamellar vesicles, FIG. 5 Panel C shows particles with multilamellar vesicles, FIG. 5 Panel D shows combined emulsion and unilamellar vesicles, and FIG. 5 Panel D shows irregular particles with lamellar structures and bridges, as well as partial emulsion particles.

It is believed that these sub-types of particles can be controlled via changes in the ingredients and processing parameters, or combinations of both. As the concentration of MCT decreases to 0% the proportion of emulsion lipid nanoparticles will decrease and the vesicle sub-type of particles will increase. This is not only true in the case of MCT, but possibly includes other oils that are a liquid at room temperature or is a liquid at room temperature when mixed with other lipids. Replacing the liquid oil at room temperature with an oil that is both solid at room temperature and waxy makes a solid lipid nanoparticle product. This type of particle will appear similar to the emulsion lipid particle because both have a dense core. Decreasing the liquid oil and/or increasing the phosphatidylcholine will likely increase the proportion of particles that are mixed or irregular. Decreasing the liquid oil and decreasing the processing pressure will increase the propensity of forming multilamellar vesicles. Decreasing the liquid oil and processing with a larger bore interaction chamber, with or without a reduction in processing pressure will increase the proportion of multilamellar vesicles.

### Example 4: Preparation of an Embodiment of the Composition

The following describes some embodiments of lipid nanoparticle powders prepared by spray drying and lyophilization. CBD isolate containing lipid nanoparticles were prepared according to the methods described in the Manufacturing Process of Example 1. In order to spray dry CBD containing lipid nanoparticles, the finished product was mixed with an additional excipient that serves as the lyoprotectant, such as 0%, 5%, 10%, 15%, or 20% of the following alone or in combination lactose, dextrose, trehalose, arginine, glycine, and/or histidine. Excipient was added to the finished product solution and mixed (200 RPM) until dissolved. Additional incubation at room temperature was allowed for material equilibration.

To spray dry the CBD lipid nanoparticles to a powder, a Buchi B290 mini benchtop spray dryer was used. The inlet temperature of the spray-dryer was set at 60-100° C. The aspirator was constant at 35 m³/hour and the feed pump varied up to 5 mL/min. Spray drying parameters were varied such that the outlet temperature was maintained at or below 65° C. and yielding a flowable powder.

To lyophilize the CBD lipid nanoparticles to a powder, a VirTis AdVantage Pro Freeze Dryer was used. Samples were placed in 20 mL glass vials with a stopper half seated. Vials were placed on the lyophilizer shelf and equilibrated at 4° C. for 6 hours before rapidly freezing at −50° C. for 12 hours. Samples were ramped to their lyophilization temperature at a rate of 0.5° C./min. After an additional 30 minutes of equilibration, primary drying commenced with the condenser set at −80° C. and chamber pressure set to 100-200 mTorr. The shelf temperature and duration of primary drying were dependent on which excipient was used, but generally were −20° C. and 24 to 36 hours, respectively. Secondary drying commenced for 6 additional hours at 25° C. and 100-200 mTorr. Following drying, vials were stoppered until further use. To produce a fine powder, samples were milled and passed successively through 75 to 34 micrometer sieves.

TABLE 2

| | | Polydispersity |
|---|---|---|
| Sample | Z-Average | Index |
| CBD Lipid Nanoparticle Solution | 125.1 nm | 0.133 |
| Reconstituted CBD Lipid Nanoparticle | 127.6 nm | 0.163 |
| Powder (5% trehalose) After 7 Months of | | |
| Controlled Room Temperature Storage | | |
| P-Value | 0.115 | 0.285 |

CBD lipid nanoparticle powders were stored in clear glass vials at 25° C./60% relative humidity for 7 months. Powders were reconstituted and particle size analysis was measured and compared to the original formulation. The original nanoparticle formulation had Z-Average particle size of 125.1 nm (average of three measurements) and the reconstituted powder have a Z-Average particle size of 127.6 nm. Statistical comparison between the two samples resulted in a p-value of 0.115. The polydispersity index of the CBD nanoparticle solution was 0.133 (average of three measurements) and the reconstituted powder had a polydispersity index of 0.163. Statistical comparison between the two samples resulted in a p-value 0.285. The results demonstrate that the CBD containing lipid nanoparticle can be reconstituted and the same particle size characteristics are preserved in the drying process. Further, since it was 7 months later, the particles are advantageously stable in powder form.

### Example 5: Preparation of an Embodiment of the Composition

The following describes an embodiment of a solvent-free approach to manufacturing an embodiment of a CBD isolate lipid nanoparticle composition. CBD lipid nanoparticles were prepared using a solvent free method utilizing a high-shear in-line mixer, followed by high pressure homogenization. All water-soluble formulation ingredients, including water soluble flavoring agents, were dissolved into water for injection at the specified concentrations. Aqueous solutions were heated and filtered prior to further use. Warm aqueous solution was transferred to a mixing vessel with an outlet at the bottom of the container that feeds the inlet of a high-shear in-line mixer (Silverson Verso Mixer). The outlet of high-shear mixer utilizes a tube that returns liquid to the top of the mixing vessel. When the warm aqueous solution is transferred to the mixing vessel, the in-line mixer is activated, and the self-pumping action of the mixer moves the liquid through the system.

Method 1. Lipophilic ingredients were accurately weighed into a glass mixing vessel and well dispersed. The lipophilic ingredients were heated with mixing to assist in the dispersion of the materials to form a homogenous lipid slurry. The lipid slurry, including any oil-based flavoring agents, was transferred slowly to the in-line mixing vessel with the mixer activated and emulsified for up to 60 minutes (in a high shear mixer).

Method 2. Lipophilic ingredients were accurately weighed onto a weigh boat and then transferred one at a time to the high-shear mixing vessel with the mixer activated. As each ingredient was introduced, 5 to 10 minutes of mixing was allowed before subsequent additions to allow for homogenous dispersion. Once all lipophilic ingredients were added, the lipid slurry was emulsified for up to 60 minutes while maintaining the processing temperature (in a high shear mixer).

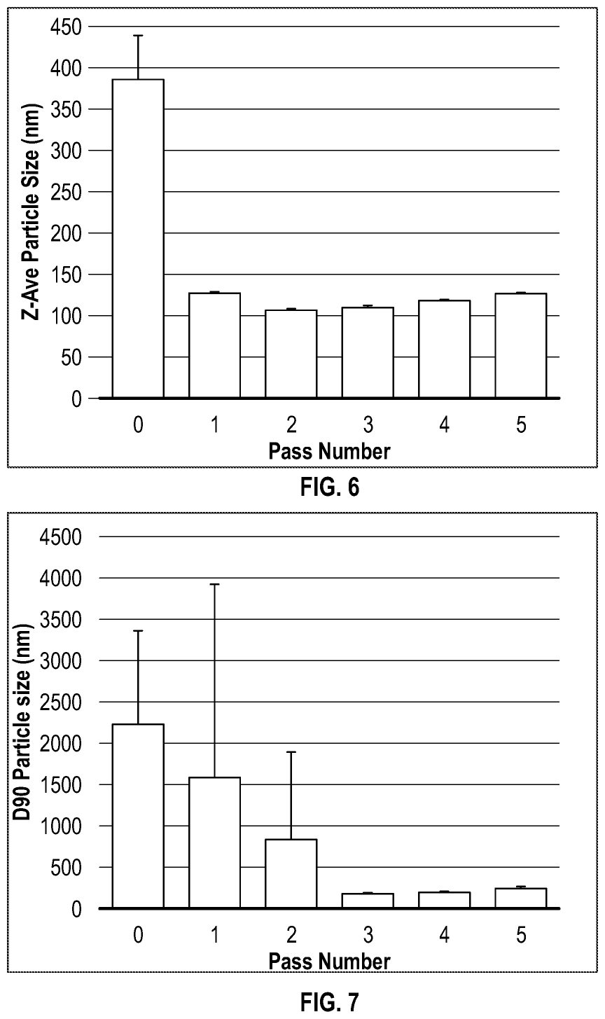

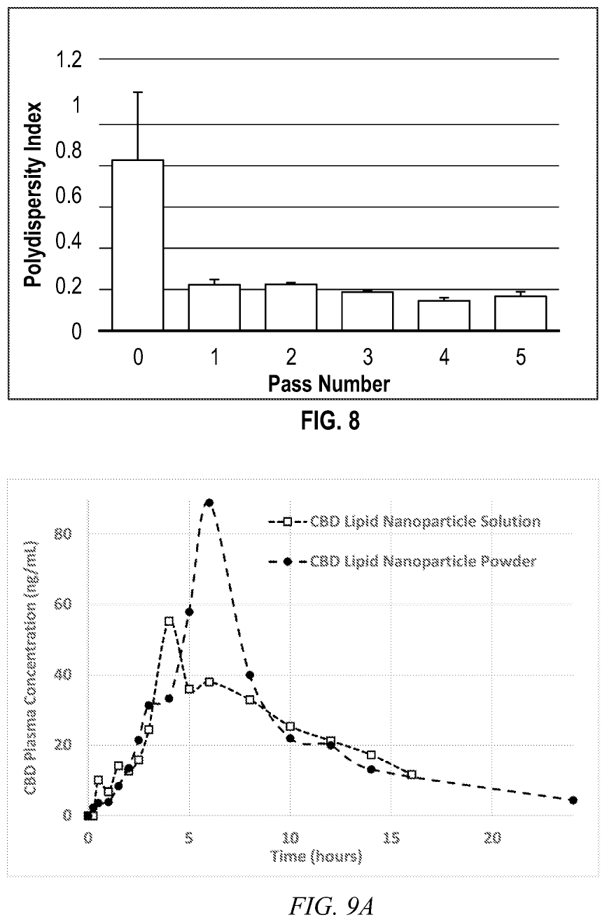

The full volume of emulsified lipid solution (as prepared in Method 1 or Method 2) was processed through a microfluidizer (Microfluidics Corporation) for 0 to 10 times at a processing pressure of 10,000-30,000 PSI. The resulting lipid nanoparticle solution was cooled with continuous stirring for a period of 12 to 24 hours before characterization and fill-finish. The data in FIGS. 6 through 8 indicate that a CBD lipid nanoparticle of appropriate particle size distribution, as characterized by the Z-Average, D90 Particle Size, and the polydispersity Index (in FIGS. 6, 7, and 8, respectively), is achieved after 60 minutes of high shear mixing and 3 full passes through a high shear homogenizer.

After the lipid slurry was emulsified for 60 minutes, the dispersion was passed through the microfluidizer 5 times and the resulting Z-Average particle size was measure after each pass (3 measurements per pass). Pass number 0 represents the particle size after high-shear mixing only and had a particle size 385.8±53.1 nm. After 1 pass through the microfluidizer, the resulting particle size decreased to 127.2±1.1 nm (n=3). After 2 and 3 passes through the microfluidizer the resulting particle size was 106.2±1.0 nm and 109.7±1.0 nm. The particle size increased slightly after passes 4 and 5 to 118.0±0.3 nm and 126.2±0.5 nm, respectively.

After the lipid slurry was emulsified for 60 minutes, the dispersion was passed through the microfluidizer 5 times and the resulting D90 particle size was measure after each pass (3 measurements per pass). The D90 particle size describes the diameter where 90% of the distribution has a smaller particle size and 10% has a larger particle size. Pass number 0 represents the particle size after high-shear mixing only and had a particle size 2,266.7±1152.4 nm. After 1 pass through the microfluidizer, the resulting particle size decreased to 1,610.0±2,364.5 nm (n=3). After 2 passes through the microfluidizer, the resulting particle size decreased to 830.3±1.083.2 nm.

After 3, 4, and 5 passes through the microfluidizer the resulting particle size was 185.0±2.0 nm, 191.3±8.4 nm, and 238.7±28.0 nm, respectively.

After the lipid slurry was emulsified for 60 minutes, the dispersion was passed through the microfluidizer 5 times and the resulting polydispersity index was measure after each pass (3 measurements per pass). Pass number 0 represents the polydispersity index after high-shear mixing only was 0.754±0.297. After 1 pass through the microfluidizer, the resulting polydispersity index decreased to 0.201±0.026 (n=3). After 2 and 3 passes through the microfluidizer the resulting polydispersity index was 0.205±0.006 and 0.172±0.002. After passes 4 and 5 the polydispersity index was 0.132±0.013 and 0.151±0.022, respectively.

### Example 6: Lipid and CBD Concentration Effect on Nanoparticle Size and Stability

CBD containing lipid nanoparticles were prepared using the solvent based manufacturing process in 100 mL batches with varied lipid concentrations to determine their impact on nanoparticle size distribution and short-term stability. Nanoparticles were aliquoted into 20 mL or greater aliquots in clear glass vessels and stored 2-8° C., 25° C. with 60% relative humidity, and 40° C. with 75% relative humidity. At regular intervals the particle size distribution was determined and Z-Average, polydispersity index, and D90 particle size was recorded. The following table summarizes percent weight of ingredients in the formulations studied.

TABLE 3

| Ingredients | F1 | F2 | F3 | F4 | F5 | F6 | F7 | F8 | F9 | F10 |
|---|---|---|---|---|---|---|---|---|---|---|
| HSPC (g) | 10 | 10 | 10 | 10 | 6 | 6 | 6 | 3 | 3 | 1 |
| Cholesterol (g) | 1 | 1 | 1 | 1 | 0.6 | 0.6 | 0.6 | 0.3 | 0.3 | 0.1 |
| MCT (g) | 9.6 | 5.76 | 2.88 | 0.96 | 5.76 | 2.88 | 0.96 | 2.88 | 0.96 | 0.96 |
| CBD (g) | 2 | 2 | 2 | 2 | 2 | 2 | 2 | 2 | 2 | 2 |
| Vitamin E (g) | 0.05 | 0.05 | 0.05 | 0.05 | 0.05 | 0.05 | 0.05 | 0.05 | 0.05 | 0.05 |
| Sodium | 0.1 | 0.1 | 0.1 | 0.1 | 0.1 | 0.1 | 0.1 | 0.1 | 0.1 | 0.1 |
| Benzoate (g) | | | | | | | | | | |
| Potassium | 0.1 | 0.1 | 0.1 | 0.1 | 0.1 | 0.1 | 0.1 | 0.1 | 0.1 | 0.1 |
| Sorbate (g) | | | | | | | | | | |
| Citric Acid | 0.1 | 0.1 | 0.1 | 0.1 | 0.1 | 0.1 | 0.1 | 0.1 | 0.1 | 0.1 |
| Monohydrate (g) | | | | | | | | | | |
| Malic Acid (g) | 0.01 | 0.01 | 0.01 | 0.01 | 0.01 | 0.01 | 0.01 | 0.01 | 0.01 | 0.01 |
| Water For | 77.04 | 80.88 | 83.76 | 85.68 | 85.28 | 88.16 | 90.08 | 91.46 | 93.38 | 95.58 |
| Injection (g) | | | | | | | | | | |

HSPC is hydrogenated sunflower phosphatidylcholine, MCT is medium chain triglyceride, and CBD is cannabidiol.

The following table summarizes the percent change of each particle size distribution parameter for each formulation after 90 days of storage at the state temperature. A negative number indicates the parameter was less than the starting measurement, where a positive number indicates the parameter was greater than the starting measurement. All numbers are the average of three measurements. NA indicates data was not available.

TABLE 4

| | 2-8° C. Storage | | | 25° C./60% RH Storage | | | 40° C./75% RH Storage | | |
|---|---|---|---|---|---|---|---|---|---|
| | Temperature | | | Temperature | | | Temperature | | |
| | Z-Ave | PDI | D90 | Z-Ave | PDI | D90 | Z-Ave | PDI | D90 |
| F1 | 5.05 | 16.11 | 14.06 | 3.03 | 4.63 | 4.45 | 27.96 | 151.01 | 159.66 |
| F2 | 8.70 | 17.74 | 32.17 | −0.18 | 30.3 | 48.6 | 41.73 | 292.44 | 124.38 |
| F3 | 30.05 | 36.95 | 185.78 | 68.18 | 85.04 | −55.47 | 416.33 | −18.46 | 75.07 |
| F4 | 228.86 | −13.88 | −32.97 | 153.56 | −29.03 | −84.06 | 104.38 | −32.79 | 3.35 |
| F5 | 14.05 | 35.40 | 915.88 | 12.98 | 40.68 | 901.81 | 24.10 | 352.55 | 102.26 |
| F6 | 172.53 | 54.75 | −71.26 | 25.38 | 96.39 | 30.88 | NA | NA | NA |
| F7 | 124.89 | 100.00 | −99.90 | 83.97 | 89.20 | −70.69 | 455.48 | 73.11 | −32.67 |
| F8 | 8.78 | 15.05 | 26.82 | 11.03 | 32.90 | 54.60 | 38.97 | 149.35 | 193.85 |
| F9 | 144.84 | 110.28 | −20.00 | 48.06 | 85.08 | −36.91 | 57.50 | 70.85 | −33.40 |
| F10 | 260.94 | 157.67 | −54.46 | −5.63 | 30.97 | 328.80 | 7.76 | 30.09 | 51.10 |

In general, CBD containing lipid nanoparticles were smaller with higher total lipid to CBD ratios, including a greater oil phase composition. A similar trend was observed with PDI, a higher total lipid to CBD ratio and higher oil content had a more homogenous particle size distribution. Following 90 days of storage at the specified storage conditions, formulations with high lipid and oil content experienced less percent change in particle size and PDI.

### Example 7: Pharmacokinetics of CBD Lipid Nanoparticle Solutions and Powders

CBD containing lipid nanoparticles were prepared according to the solvent based manufacturing process using formulation ingredients outlined in batches 2 & 4 in Example 1. Powders of the CBD containing lipid nanoparticles were prepared according to the methods outlined in Example 4.

The pharmacokinetics of liquid and powder in capsule lipid formulations of CBD were determined in male Gottingen mini-pigs at a dose of 15 mg/kg. Mini-pigs (20-24 kg) were orally administered the product into the stomach by an oral gavage tube. Blood samples were collected via an accessible vein into blood tubes containing potassium EDTA. Blood samples were collected at 0 (pre-dose), 0.25, 0.5, 1, 1.5, 2, 2.5, 3, 4, 5, 6, 8, 10, 12, 14, and 16 or 24 hours. CBD concentration and metabolites were measured in blood plasma by HPLC. Pharmacokinetic parameters were determined from the plasma concentrations using PK Solver, a Microsoft Excel plug-in, or by hand using the linear trapezoid rule. For comparison, leading commercially available, oil-based CBD products were also evaluated after oral administration.

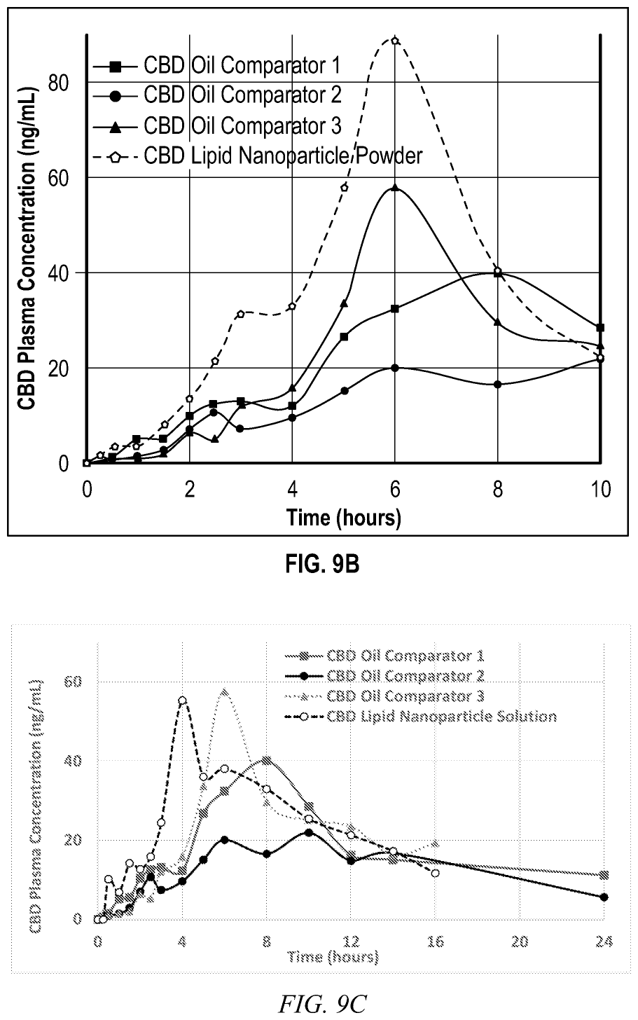

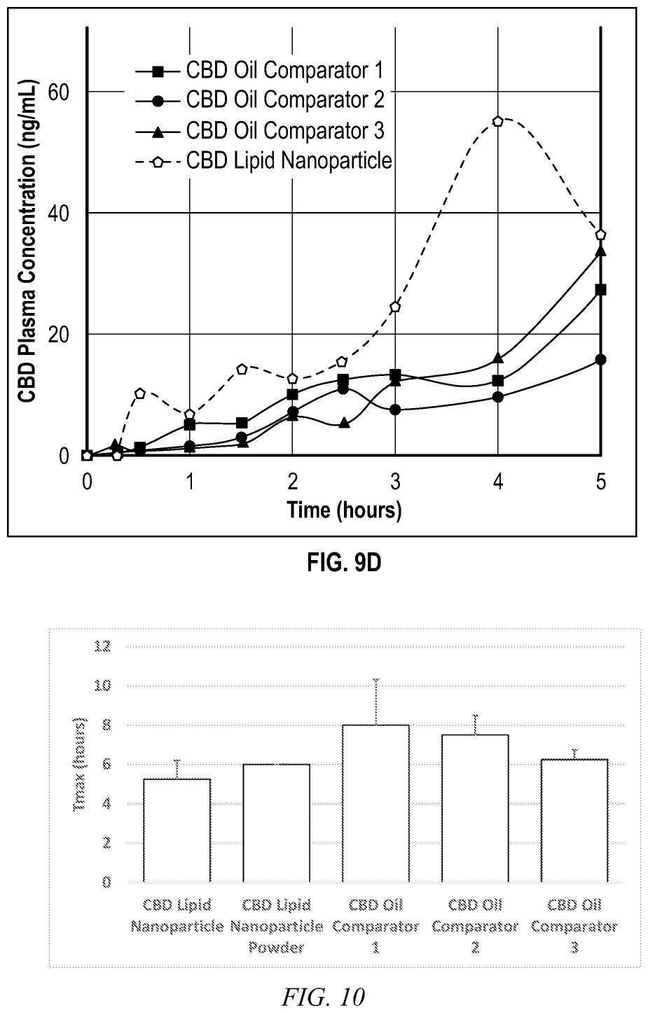

Shown in FIG. 9A-D are the pharmacokinetic profiles of CBD containing lipid nanoparticles in solution as well the powder formulation filled in gelatin capsules. FIG. 9A shows two embodiments as disclosed herein. As demonstrated, the nanoparticle powder had an increased Cmax and the solution had an increased Tmax. FIG. 9B shows that the powder formulation in gelatin capsules had a Cmax that was approximately 63% higher than the CBD-oil comparators. As shown in FIGS. 9C and 9D, the solution formulation had faster Tmax (˜4 hours) compared to the CBD-oil comparators, which had a Tmax of greater than 6 hours and close to 8 hours in some samples. FIGS. 9C and 9D show the CBD lipid nanoparticle solution had detectable concentrations of CBD earlier than the oil-based comparators (within the first hour of the study), as well as reached an apparent Tmax approximately 2 hours earlier than oil-based comparator 3, 4 hours earlier than oil-based comparator 1, and 6 hours earlier than oil-based comparator 2. The CBD lipid nanoparticles reached a higher plasma concentration than comparators 1 and 2.

Shown in FIG. 10 is the comparison of the absorption phase of the CBD lipid nanoparticle solutions over the first four hours of the study, and three leading oil-based CBD commercial comparators. For the CBD lipid nanoparticles, measurable levels of CBD were detected in plasma within 30 minutes. The rates of absorption were taken to be the slope of the regression equation. The rate of the absorption for the CBD lipid nanoparticle solution was statistically significant compared to the CBD oil-based comparators (ANOVA, p=0.0417).

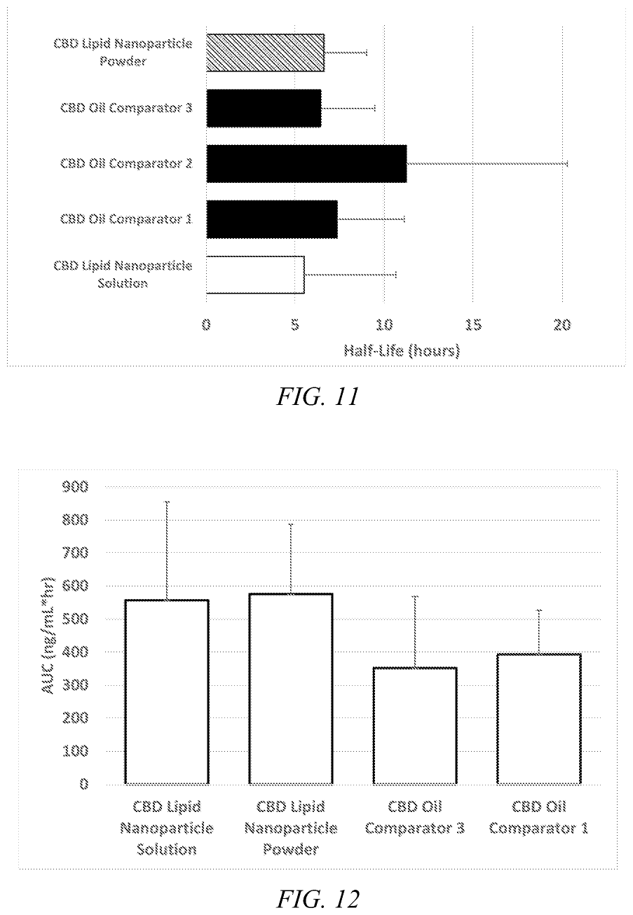

The CBD lipid nanoparticle solution formulation had the shortest half-life of 5.5±5.2 hours and the CBD lipid nanoparticle powder formulation having a half-life of 6.6±2.4 hours (FIG. 11). The CBD oil-based comparators had half-lives generally greater than the liquid formulation, of 6.4±3.0, 11.2±9.1, and 7.3±3.8 hours.

| | AUC₀₋₄ | AUC₄₋₆ | AUC₆₋₁₀ | AUC_{0-inf} |
|---|---|---|---|---|
| | (ng/ | (ng/ | (ng/ | (ng/ |
| Group | mL*hr)* | mL*hr)* | mL*hr)* | mL*hr)** |
| CBD Lipid | 98.4 ± | 84.0 ± | 129.4 ± | 557.8 ± |
| Nanoparticle | 45.2 | 64.3 | 31.5 | 297.5 |
| CBD Lipid | 65.8 ± | 119.0 ± | 191.0 ± | 575.9 ± |
| Nanoparticle | 25.5 | 12.9 | 58.1 | 211.5 |
| Powder | | | | |
| CBD Oil | 21.9 ± | 28.2 ± | 70.7 ± | 393.8 ± |
| Comparator 1 | 20.2 | 20.9 | 36.0 | 133.0 |
| CBD Oil | 33.7 ± | 49.2 ± | 141.2 ± | 352.1 ± |
| Comparator 2 | 26.9 | 21.2 | 45.3 | 216.9 |
| CBD Oil | 24.7 ± | 84.0 ± | 141.9 ± | 568.7 ± |
| Comparator 3 | 16.1 | 64.3 | 64.5 | 311.1 |

FIG. 12 shows AUC or Area Under the Curve information (0 to infinity). AUC is a pharmacokinetic parameter that reflects a molecule's total exposure. The CBD lipid nanoparticle solution had an AUC of 557.8±297.5 ng/mL*hr, where the CBD lipid nanoparticle powder had an AUC of 575.9±211.5 ng/mL*hr. Despite having a significantly greater Cmax (shown in FIG. 9), both liquid and powder formulations had comparable AUCs. Both oil-based CBD comparators had AUCs that were lower than the Lipid Nanoparticle formulation. Comparator 3 had an AUC of 352.1±216.9 and comparator 1 had an AUC of 393.8±133.0 ng/mL*hr. Indicating the oil-based CBD products had less total exposure than the lipid nanoparticle formulations.

Shown in Table 5 are the AUCs for 0-4 hr and 0—infinity hours (0—infinity calculated using PKSolver, the rest were calculated using the linear trapezoid equation). The AUC₀₋₄ for the CBD lipid nanoparticles and powder was 98.4±45.2 and 65.8±25.5 ng/mL*hr, respectively. The CBD oil comparators had AUCs for this same period of time were 21.9±20.2, 33.7±26.9, and 24.7±16.1 ng/mL*hr. The AUC₄₋₆ for the CBD lipid nanoparticles and powder was 84.0±64.3 and 119.0±12.9 ng/mL*hr, respectively. The AUCs for this same period of time were 28.2±20.9, 49.2±21.2, and 84.0±64.3 ng/mL*hr for the oil based comparators. The AUC₆₋₁₀ for the CBD lipid nanoparticles and powder was 129.4±31.5 ng/mL*hr and 191.0±58.1 ng/mL*hr. The AUCs for the CBD oil based comparators were 70.7±36.0, 141.2±45.3, and 141.9±64.5 ng/mL*hr over the same period of time. The higher AUCs during the first 4 hours of the study in the CBD lipid nanoparticle groups demonstrate the rapid absorption compared to the oil-based comparators.

TABLE 5

| | AUC0-4 | AUC0-inf |
|---|---|---|
| Group | (ng/mL*hr)* | (ng/mL*hr)** |
| CBD Lipid Nanoparticle | 98.4 ± 45.2 | 557.8 ± 297.5 |
| CBD Lipid Nanoparticle | 65.8 ± 25.5 | 575.9 ± 211.5 |
| Powder | | |
| CBD Oil Comparator 1 | 21.9 ± 20.2 | 393.8 ± 133.0 |
| CBD Oil Comparator 2 | 33.7 ± 26.9 | 352.1 ± 216.9 |
| CBD Oil Comparator 3 | 24.7 ± 16.1 | 568.7 ± 311.1 |

*Calculated by hand/Excel using the linear trapezoid rule
**Calculated by PKSolver for Excel

### Example 8: Preservative Systems of CBD Containing Lipid Nanoparticles

CBD containing lipid nanoparticles were prepared using the solvent based manufacturing process, however, different concentrations of preservatives were dissolved in the aqueous solution prior to hydration of the lipid film and mixing. Citric acid monohydrate and malic acid was added to Formulation 1 at 6.10 and 5.73 mM, respectively. In Formulation 2, citric acid was added at 4.88 mM and no malic acid was added. In Formulation 3, citric acid was added 0.16 mM and no malic acid was added. In Formulation 4, no citric or malic acid was added. All formulations contained 8.53 mM of potassium sorbate and 8.90 mM of sodium benzoate. Formulations were characterized for pH, particle size distribution, zeta potential, CBD concentration and particle size after storage for 6 or 7 months at 2-8° C., 25° C. with 60% relative humidity, and 40° C. with 75% relative humidity, and a preservative effectiveness challenge. The table below summarizes Formulation initial characterization data.

TABLE 6

| | Solution | Z-Average | D90 | Polydispersity | Zeta |
|---|---|---|---|---|---|
| Form. | pH | Particle Size | Particle Size | Index | Potential |
| 1 | 4.072 | 102.3 ± 0.61 nm | 146.3 ± 3.61 nm | 0.164 ± 0.005 | +2.29 mV |
| 2 | 4.459 | 103.2 ± 0.94 nm | 149.0 ± 4.16 nm | 0.174 ± 0.019 | +3.25 mV |
| 3 | 5.093 | 103.3 ± 0.85 nm | 149.0 ± 3.21 nm | 0.166 ± 0.007 | +3.00 mV |
| 4 | 6.250 | 99.8 ± 1.35 nm | 135.7 ± 4.01 nm | 0.156 ± 0.025 | +1.82 mV |

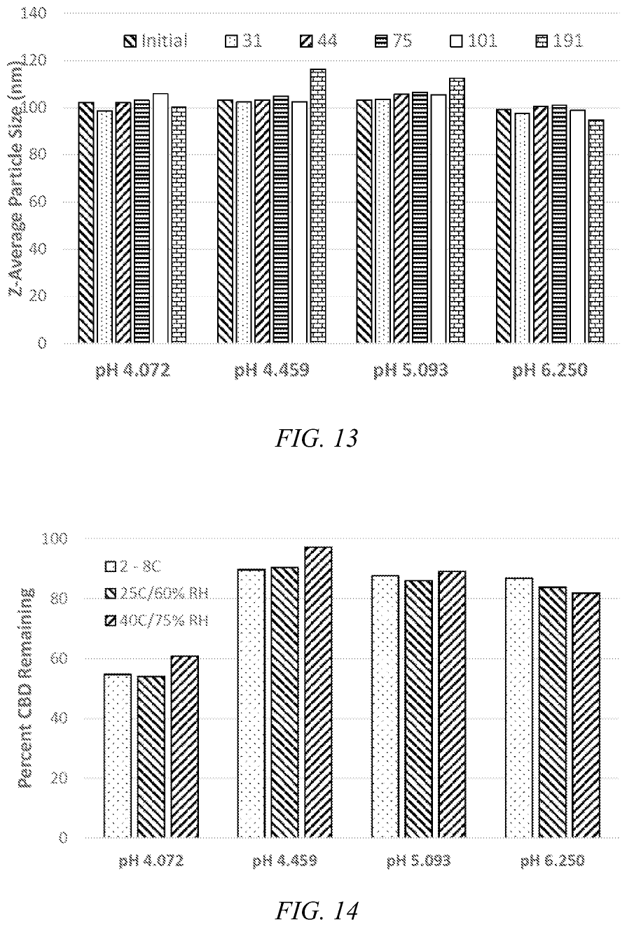

FIG. 13 shows the change in CBD lipid nanoparticle size over approximately 6 months at differing solution pH values. FIG. 14 shows the change in CBD concentration in lipid nanoparticles over approximately 7 months at differing storing conditions. Solution pH did not impact the stability of the particle size (FIG. 13) when measure at regular intervals over approximately 6 months of storage at 25° C. with 60% relative humidity. After 7 months of storage at 2-8° C., 25° C. with 60% relative humidity, and 40° C. with 75% relative humidity, the percent CBD remaining was significantly less for pH 4.072 compared to the formulation groups.

To determine the effectiveness of the preservative system, the formulations were challenged with 5 microorganisms (E. coli, P. aeruginosa, S. aureus, A. brasiliensis, and C. albicans) at 10⁷ CFU/mL and the log reduction in colony forming units after incubation for 7 days was calculated.

TABLE 7

| | E. | P. | S. | A. | C. |
|---|---|---|---|---|---|
| Formulation pH | coli | aeruginosa | aureus | brasiliensis | albicans |
| Formulation 1 | >4.18 | >4.30 | >4.08 | >1.75 | >1.00 |
| Formulation 2 | >4.18 | >4.30 | >4.08 | 1.63 | 1.00 |
| Formulation 3 | 0.37 | >4.30 | >4.08 | 0.12 | None |
| Formulation 4 | 1.03 | None | 0.74 | None | 0.07 |

The minimum require for an effective preservative system is at least a 1.0 log reduction in colony forming units for each organism evaluated after 7 days of incubation. Preservative systems with a pH of 4.459 and 4.07s met the minimum requirements of a preservative system, but solutions with a pH of 5.093 and 6.250 did not. The preservative systems evaluated in this study were more effective at preventing bacterial growth, especially at lower pH, than yeasts and molds.

### Example 9: Higher Concentrations of CBD in the Lipid Nanoparticle Formulation

CBD containing lipid nanoparticles were prepared using the solvent based manufacturing process outlined above. In this example, the lipid ratios were fixed with respect to each other and the CBD concentration was varied. Formulation compositions are outlined in the table below. Formulations were stored at 2-8° C., 25° C. with 60% relative humidity, and 40° C. with 75% relative humidity for 100 days and the particle size distribution was determined. Results reported are the average percent change from the initial conditions recorded on day 0 (n=3 measurements per sample, per time point). A positive number indicates the particle size parameter increased with respect to day 0, while a negative number indicates the parameter decreased with respect to day 0.

TABLE 8

| | Percent | Percent | Percent |
|---|---|---|---|
| | CBD By | Lipid By | Water By |
| | Weight | Weight | Weight |
| Formulation No. 29 | 3.00 | 20.67 | 76.32 |
| Formulation No. 30 | 4.00 | 20.67 | 75.32 |
| Formulation No. 31 | 5.00 | 20.67 | 74.32 |
| Formulation No. 32 | 2.00 | 12.42 | 85.58 |
| Formulation No. 33 | 3.50 | 12.42 | 84.07 |
| Formulation No. 34 | 6.00 | 12.42 | 81.57 |
| Formulation No. 36 | 4.00 | 12.42 | 83.57 |

The table below summarizes the results of the study as percent change in Z-average particle size and polydispersity index after 100 days of storage at the stated storage temperature. Despite the percent change in particle size parameters at any storage temperature, all were within the product's specification, indicating that CBD can be incorporated into the formulation beyond 2%.

TABLE 9

| | 2-8° C. | | 25° C./60% | | 40° C./75% | |
|---|---|---|---|---|---|---|
| | Storage | | RH Storage | | RH Storage | |
| | Temperature | | Temperature | | Temperature | |
| Formulation | Z-Ave | PDI | Z-Ave | PDI | Z-Ave | PDI |
| Formulation No. 29 | 7.36 | 13.71 | 4.25 | 35.05 | 71.95 | 57.14 |
| Formulation No. 30 | 7.71 | 5.17 | 7.28 | 54.87 | 71.25 | 54.27 |
| Formulation No. 31 | 6.94 | −1.8 | 11.95 | 101.00 | 51.70 | 46.00 |
| Formulation No. 32 | 7.02 | 2.46 | 7.60 | 49.08 | 80.36 | 44.36 |
| Formulation No. 33 | 14.49 | 23.23 | −1.23 | 38.38 | 70.79 | 96.21 |
| Formulation No. 34 | 6.26 | 8.64 | 8.32 | 120.74 | 64.6 | 90.37 |
| Formulation No. 36 | NA | NA | 10.63 | 90.78 | 57.13 | 40.67 |

### Example 10: CBD Containing Lipid Nanoparticles Can Be Filtered

CBD containing lipid nanoparticles were prepared using the solvent based method at a 10 liter batch size. Prior to further study, the nanoparticles were characterized for particle size distribution and CBD concentration. To filter the material, the nanoparticle solution was transferred to a pressurized vessel containing a stainless-steel side arm. To the side arm, Pharmed BPT tubing was used to connect the pressurized vessel to a receiving vessel, with a 3M betafine filter in-line. To filter the nanoparticle solution, nitrogen gas was filled into the pressurized vessel to displace the solution forcing it through the filter and into the receiving vessel. Two 3M betafine filters were evaluated in this study, a 0.2 micron and 0.65 micron polypropylene filter. After filtration, the particle size distribution and CBD concentration was measured again and compared to the starting measurements. All measurements were performed in triplicate.

TABLE 10

| | | | After 3M | | After 3M | |
|---|---|---|---|---|---|---|
| | | | Betafine | | Betafine | |
| | Starting | | 0.20 Micron | | 0.65 Micron | |
| Parameter | Measurements | | Filter | | Filter | |
| Z-Average | 103.5 | nm | 101.5 | nm | 101.2 | nm |
| Particle Size | | | | | | |
| Polydispersity Index | 0.184 | | 0.123 | | 0.154 | |
| D90 Particle Size | 179.0 | nm | 151 | nm | 155.7 | nm |
| CBD Concentration | 20.0 | mg/mL | 20.0 | mg/mL | 20.0 | mg/mL |

No change in the particle size parameters and CBD concentration before and after filtration indicates the product can be filtered at a 0.2 micron cutoff without any loss of material. Further indicating the product may be sterile-filtered through a 0.22 micron sterile filter.

### Example 11: Resulting Particle Size Distribution By Operating Pressure And Pass Number

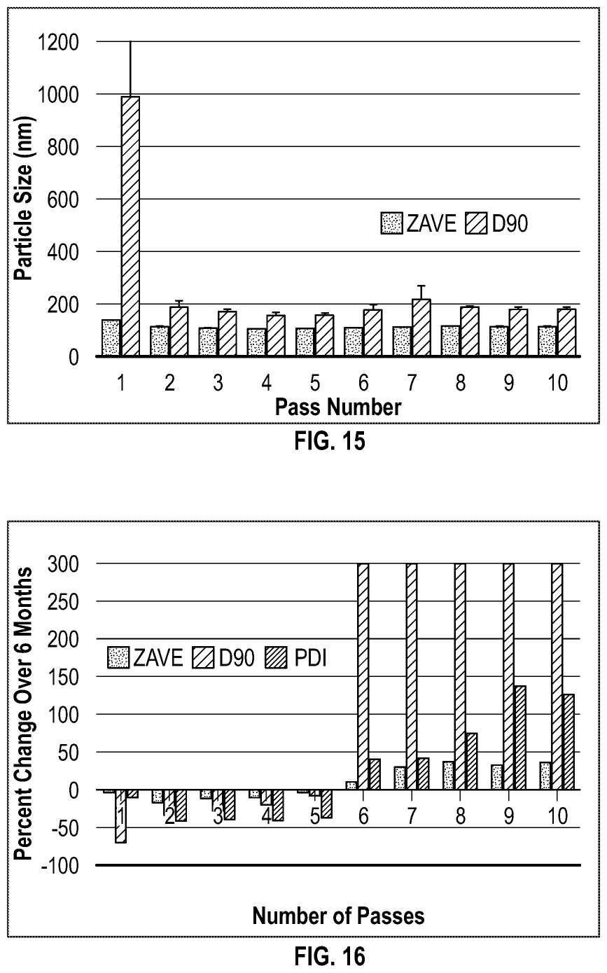

CBD containing lipid nanoparticles were prepared by the solvent based manufacturing process in batch sizes of 100 mL. The purpose of the first part of the study was to determine the impact of pass number on the initial particle size distribution and any changes after 6 months of storage at 25° C. with 60% relative humidity. The full volume of lipid slurry was microfluidized 10 times with a sample collection after each volume for analysis. Shown below in FIG. 15 is the Z-Average and D90 particle sizes. After 1 pass through the microfluidizer, the Z-Average was below 200 nm but the D90 particle size was 1.0 micron. After 2 passes through the microfluidizer both the Z-Average and D90 were below 200 nm. The difference between the particle sizes decreased with subsequent passes up to pass 5. Starting with and after pass 6, the two particle sizes increased in difference. Interestingly, the percent change in particle size parameters decreased slightly after 6 months of storage at 25° C. with 60% relative humidity for passes 1 through 5 (FIG. 16). However, significant increases in the D90 and PDI were observed for passes 6 through 10 during the same storage conditions and time. The D90 particle size increased by at least 300% for passes 6 through 10.

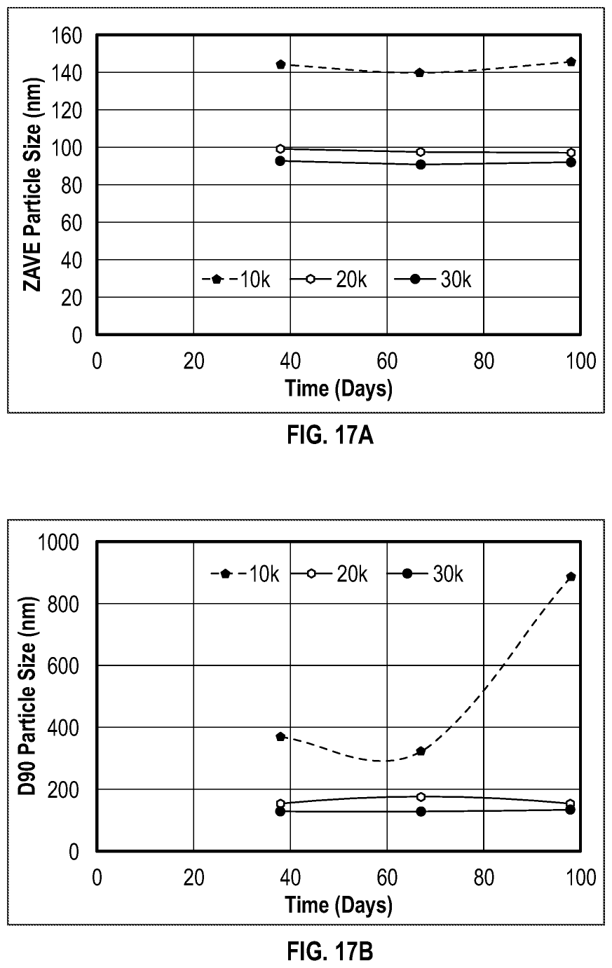

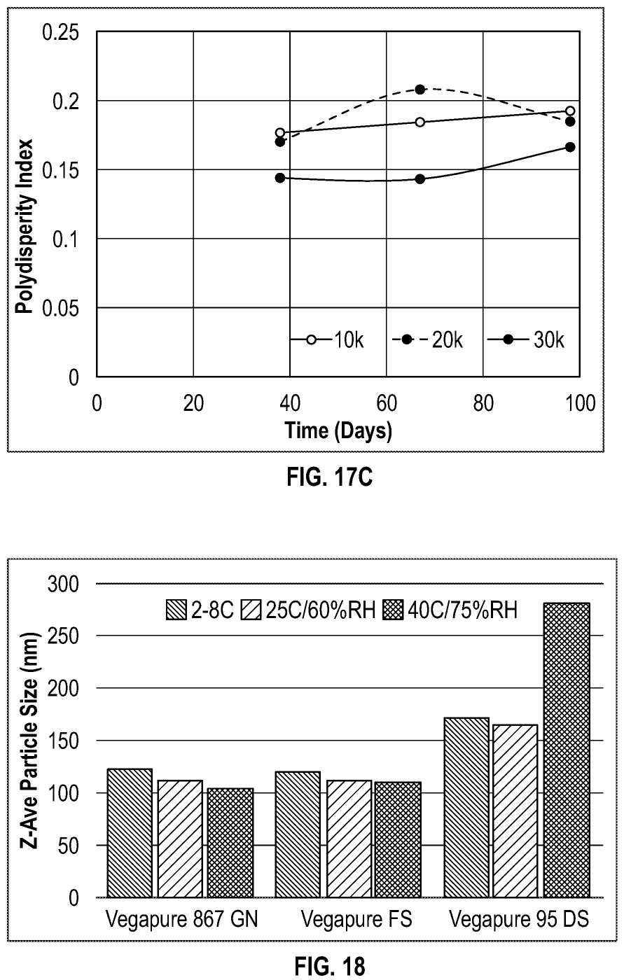

In the second part of this study, batches of CBD containing lipid nanoparticles were prepared at different microfluidizer operating pressures and the impact on the particle size distribution was measure over 90 days of storage at 25° C. with 60% relative humidity. Shown below in FIG. 17A-C is the Z-Ave, D90 particle sizes, and polydispersity index, respectively, by operating pressure. The Z-Ave particle size decreased with increasing operating pressure, with the most dramatic difference being between 10,000 and 20,000 PSI. During the 90-day storage period the Z-Ave particle size did not significantly change with any operating pressure. A similar trend was observed with the D90 particle size. However, the batch prepared at 10,000 PSI showed a significant increase in particle size at day 90 compared to the 20,000 and 30,000 PSI operating pressures. The difference in polydispersity index wasn't as dramatic as particle size and didn't change over 90 days (the −70 day measurement at 20,000 PSI being an exception).

### Example 12: CBD Containing Lipid Nanoparticles Prepared With Several CBD Isolates

CBD containing lipid nanoparticles were prepared using the solvent based manufacturing process or the solvent free, high shear mixing process in 100 mL batches. Lipid nanoparticles were prepared with CBD isolate from different manufacturers, all of which had greater than 99% CBD purity and no detectable THC. Nanoparticles were prepared at 20 mg/mL and the final concentration was verified by UHPLC. All preparations had a Z-average particle size between 85.4 nm and 105.6 nm, a D90 particle size of 113.0 nm to 153.2 nm, and a polydispersity index of 0.105 to 0.169. Lipid nanoparticles prepared with Gen Canna, Global Cannabinoids, and Mile High Labs CBD isolate was not significantly different from that prepared with Boulder Botanicals CBD isolate, indicating similar nanoparticle attributes are attainable regardless of the CBD isolate origin. The results of this example are summarized in the table below.

TABLE 11

| | Percent | Percent | | | |
|---|---|---|---|---|---|
| | CBD | THC | Z-Ave | D90 | Polydis- |
| Manufac- | Compo- | Compo- | Particle | Particle | persity |
| turer | sition | sition | Size | Size | Index |
| Boulder | 99.97% | Not Detected | 104.4 nm | 151.0 nm | 0.158 |
| Botanicals | | | | | |
| Gen Canna | >99% | Not Detected | 105.6 nm | 153.2 nm | 0.169 |
| Global | 99.93% | Not Detected | 85.4 nm | 113.0 nm | 0.105 |
| Cannabinoids | | | | | |
| Mile High | 99.30% | Not Detected | 94.84 nm | 131.0 nm | 0.129 |
| Labs | | | | | |

### Example 13: CBD Containing Lipid Nanoparticles Prepared With Full Or Broad Spectrum CBD Material

CBD containing lipid nanoparticles were prepared by the solvent based and/or solvent free manufacturing process in 0.1 liter batches. In this example, the CBD origin was from a full spectrum or broad spectrum hemp extract where the CBD content varied from 44.25% to 86.6%. The THC content was below 0.3% or not detectable. All formulations were prepared to a final concentration of 20 mg/mL CBD and confirmed by UHPLC. Modifications to the remaining lipids in the formulations were made to accommodate the lower concentration of CBD in the full/broad spectrum hemp extracts. All formulations had a Z-average particle size between 94.88 nm and 178.0 nm, a D90 particle size between 132.0 nm and 265.0 nm, and a polydispersity index of 0.100 to 0.221. The resulting particle size attributes were not different from those prepared with CBD isolate, indicating the broad or full spectrum CBD can be exchanged with CBD isolate in the lipid nanoparticle formulation. The results of this study are summarized in the table below.

TABLE 12

| | Percent | Percent | | | |
|---|---|---|---|---|---|
| | CBD | THC | Z-Ave | D90 | Polydis- |
| Manufac- | Compo- | Compo- | Particle | Particle | persity |
| turer | sition | sition | Size | Size | Index |
| Boulder | | | 94.88 nm | 132.0 nm | 0.152 |
| Botanicals | | | | | |
| Full Spectrum | | | | | |
| CBD Extract | | | | | |
| Klersun NDT | 83.16% | <0.3% | 98.15 nm | 138.0 nm | 0.138 |
| Broad | | | | | |
| Spectrum | | | | | |
| Hemp Extract | | | | | |
| Mile High | 86.6% | Not Detected | 98.87 nm | 193.0 nm | 0.221 |
| Labs Broad | | | | | |
| Spectrum | | | | | |
| THC Free | | | | | |
| Distillate | | | | | |
| Charlotte's | 44.25% | <0.3% | 178.0 nm | 265.0 nm | 0.100 |
| Web Hemp | | | | | |
| Oil | | | | | |
| Concentrate | | | | | |

### Example 14: Lipid Nanoparticles Prepared with CBG Isolate, CBN Distillate, and CBDa Oil

Lipid nanoparticles were prepared with other commercially available cannabinoids using the solvent based manufacturing process and characterized for particle size distribution. Global cannabinoids CBG isolate had 93.34% CBG by weight, with no other cannabinoids detected (based on Manufacturer's COA). The Z-average particle size was 105.6 nm, the D90 particle size was 241.0 nm, and the polydispersity index was 0.206. Lipid nanoparticles were prepared with CBN distillate from global cannabinoids. The CBN distillate was 80.5% CBN by weight, contained 3.1% CBC by weight, but no other cannabinoids were detectable (based on Manufacturer's COA). The Z-average particle size was 99.59 nm, the D90 particle size was 139.0 nm, and the polydispersity index was 0.138. Lipid nanoparticles were also prepared using a dilute CBDa oil (Myriam's Hope, Nev.) with not modification to the formulation lipid ratios (results not shown). The results of the CBG and CBN nanoparticles are summarized in the table below.

TABLE 13

| | | Z-Ave | D90 | |
|---|---|---|---|---|
| | Cannabinoid | Particle | Particle | Polydispersity |
| Cannabinoid | Composition | Size | Size | Index |
| Global | CBG: 93.34% | 105.6 nm | 241.0 nm | 0.206 |
| Cannabinoids | CBD: Not | | | |
| CBG Isolate | detected | | | |
| | THC: Not | | | |
| | detected | | | |
| Global | CBN: 80.5% | 99.59 nm | 139.0 nm | 0.138 |
| Cannabinoids | CBC: 3.1% | | | |
| CBN | CBD: Not | | | |
| Distillate | detected | | | |
| | THC: Not | | | |
| | detected | | | |

### Example 15: Phytosterol Alternatives To Cholesterol Used To Prepare CBD Containing Lipid Nanoparticles

CBD lipid nanoparticle formulations were prepared using the solvent based manufacturing process in 0.1 liter batches. In this example, formulations were prepared with different phytosterols as alternatives to cholesterol. The physterosterols were purchased from BASF corporation and named Vegapure 867 GN, Vegapure FS, and Vegapure 95DS. The phytosterol replaced cholesterol in the formulation at the same weight percent, no additional modifications were made to the formulation, no cholesterol was added. The table below summarizes the initial particle size measurements using the three phytosterol alternatives to cholesterol. The Vegapure 867 GN had a Z-average particle size of 85.1 nm and PDI of 0.152, the Vegapure FS had a Z-average particle size of 87.6 nm and PDI of 0.168, the Vegapure 95 DS had a particle size of 130.7 nm and PDI of 0.400.

TABLE 14

| BASF Vegapure 867 GN | | BASF Vegapure FS | | BASF Vegapure 95 DS | |
|---|---|---|---|---|---|
| Z-Ave | PDI | Z-Ave | PDI | Z-Ave | PDI |
| 85.1 ± | 0.152 ± | 87.6 ± | 0.168 ± | 130.7 ± | 0.400 ± |
| 0.3 nm | 0.008 | 0.5 nm | 0.004 | 3.4 nm | 0.042 |

In a preliminary, short-term stability study, formulations prepared with BASF Vegapure phytosterol were placed at 2-8° C., 25° C. with 60% relative humidity, and 40° C. with 75% relative humidity for 14 days. Formulations prepared with Vegapure 867 GN and Vegapure FS had Z-average particle sizes at or below 130.0 nm for all storage temperatures. The formulation prepared with Vegapure 95 DS had particle sizes above 150.0 nm when stored at 2-8° C. and 25° C. with 60% relative humidity, but the particle size increased to above 250 nm when stored at 40° C. with 75% relative humidity. The results are shown in FIG. 18.

### Example 16: Preparing CBD Lipid Nanoparticles With Alternatives To Medium Chain Triglycerides

Part 1: CBD lipid nanoparticles were prepared using the solvent based manufacturing process at 0.1-liter batches. The medium chain triglycerides (MCT) were replaced with alternatives available from ABITEC Corporation. Captex 8000 NF is triglyceride of caprylic acid, Captex GTO is a triglyceride of oleic acid, and Captex 1000 is a triglyceride of capric acid. The Captex triglycerides replaced the MCT in the weight percents stated in the table below. The table also summarizes the initial particle size and polydispersity index.

TABLE 15a

| | Initial | | Initial |
|---|---|---|---|
| | Z-Average | | Polydispersity |
| Formulation | Particle Size | | Index |
| 5% of ABITEC Captex 8000 NF | 111.3 ± 0.61 | nm | 0.216 ± 0.005 |
| 10% of ABITEC Captex 8000 NF | 102.8 ± 2.05 | nm | 0.194 ± 0.011 |
| 10% of ABITEC Captex GTO | 92.0 ± 0.98 | nm | 0.117 ± 0.016 |
| 5% of ABITEC Captex GTO | 110.4 ± 0.51 | nm | 0.280 ± 0.018 |
| 5% of ABITEC Captex 1000 | 105.3 nm | | 0.180 |

CBD lipid nanoparticles were prepared using the solvent based manufacturing process at 0.1 liter batches. The medium chain triglycerides (MCT) were replaced with alternative non-aqueous liquids including omega-3 fatty acids (Tonalin and Pronova Pure® 46:38), glyceryl monooleate, conjugated linoleic acid, and alpha glycerylphosphorylcholine (alpha-GPC). The ingredients replaced MCT with an equivalent weight (10%) as presented in the original formulation. The table below summarizes the formulations and the initial particle size measurements.

TABLE 15b

| | Initial | | Initial |
|---|---|---|---|
| | Z-Average | Initial | D90 |
| | Particle | Polydispersity | Particle |
| Formulation | Size | Index | Size |
| Tonalin | 89.8 nm | 0.097 | 120.0 nm |
| Pronova Pure 46:38 | 81.8 nm | 0.084 | 106.0 nm |
| Glyceryl Monooleate | 104.8 nm | 0.114 | 152.0 nm |
| Conjugated Linoleic Acid | 244.2 nm | 0.159 | 410.0 nm |
| Alpha-GPC | 85.6 nm | 0.08 | 117.0 nm |

The table below shows the percent change in Z-average and polydispersity index when stored at 40° C. with 75% relative humidity for 30 days. A negative number indicates the particle size or PDI measurement decreased with respect to the initial measurements shown in the table above.

TABLE 16

| | Percent Change in | Percent Change in |
|---|---|---|
| | Z-Ave Particle | Polydispersity |
| | Size After 30 | Index After 30 |
| | Days Storage At | Days Storage At |
| Formulation | 40 C./75% RH | 40 C./75% RH |
| 5% of ABITEC Captex 8000 NF | −13.92% | −25.93% |
| 10% of ABITEC Captex 8000 NF | −15.61% | −46.39% |
| 10% of ABITEC Captex GTO | −5.80% | −13.07% |
| 5% of ABITEC Captex GTO | −12.35% | −21.69% |
| 5% of ABITEC Captex 1000 | −13.06% | −18.90% |

### Example 17: Preparation of an Embodiment of the Composition

A composition for the delivery of CBD was prepared using the following method. To prepare the composition, CBD (2.0 g) was dissolved in medium chain triglyceride (9.3 g) with mixing. To this solution was added, cholesterol (1.0 g) and phosphatidylcholine (10.0 g). Vitamin E was added (0.05 g) with stirring and to act as an antioxidant in the oil phase. At that time, malic acid (0.085 g), citric acid (0.085 mg), potassium sorbate (0.1 g), sodium benzoate (0.1 g), and Monk Fruit Extract (0.09 g) was added to water (76.07 g) with mixing. The aqueous phase was added to the oil phase with mixing.

Next, the oil-in-water emulsion was processed to a nanoparticle (about 20-500 nm) by successively passing the solution through microfluidizer (5 times at 30,000 PSI) at a temperature of at least 65° C. The microfluidizer contained an interaction chamber consisting of 50 to 70 um pore sizes.

### Example 18: Preparation of an Embodiment of the Composition

A composition for the delivery of CBD was prepared using the following method. To 100 ml of ethanol was added CBD isolate (2.0 g) comprising not more than 0.3% THC by weight per weight (w/w). At that time, medium chain triglyceride (9.3 g) was added with mixing. To this solution was added, cholesterol (1.0 g), phosphatidylcholine (10.0 g), and Vitamin E (0.05 g).

Next, the solvent was removed to prepare a dried composition. An oil-in-water emulsion was prepared by suspending the dried composition with 76.07 g of warm water containing malic acid (0.085 g), citric acid (0.085 mg), potassium sorbate (0.1 g), sodium benzoate (0.1 g), and Monk Fruit extract (0.09 g). The oil-in-water emulsion was processed to a nanoparticle (20-500 nm) by successively passing the solution through microfluidizer 5 times at 30,000 PSI at a temperature of at least 75° C. The microfluidizer contained an interaction chamber consisting of 50 to 70 um pore sizes.

### Example 19: Testing of an Embodiment of the Composition

A 5 Liter manufacturing batch was analyzed by high pressure liquid chromatography (HPLC) to measure cannabinoids present in the sample. The results were as shown in the following table:

TABLE 17

| Cannabinoid | LOQ (%) | Mass (%) | Mass (mg/g) |
|---|---|---|---|
| THCa | 0.01 | ND | ND |
| Δ9-THC | 0.01 | ND | ND |
| Δ8-THC | 0.01 | ND | ND |
| CBD | 0.01 | 2.12 | 21.2 |
| CBDa | 0.01 | ND | ND |
| CBC | 0.01 | ND | ND |
| CBG | 0.01 | ND | ND |
| CBN | 0.01 | ND | ND |
| THCV | 0.01 | ND | ND |
| CBGa | 0.01 | ND | ND |
| TOTAL | | 2.12 | 21.2 |

A 5 Liter manufacturing batch was analyzed by high pressure liquid chromatography (HPLC) to measure Terpenes present in the sample. The results were as shown in the following table:

TABLE 18

| Analyte | LOQ (%) | Mass (%) | Mass (mg/g) |
|---|---|---|---|
| α-Bisabolol | 0.05 | ND | ND |
| α-Humulene | 0.05 | ND | ND |
| α-Pinene | 0.05 | ND | ND |
| β-Caryophyllene | 0.05 | ND | ND |
| β-Pinene | 0.05 | ND | ND |
| Caryophyllene Oxide | 0.05 | ND | ND |
| δ-Limonene | 0.05 | ND | ND |
| Linalool | 0.05 | ND | ND |
| Ocimene | 0.05 | ND | ND |
| Terpinolene | 0.05 | ND | ND |
| Trans-Nerolidol | 0.05 | ND | ND |

ND is not detected, below LOQ

### Example 20. Noopept and CBD Lipid Nanoparticle Formulations

The solvent based lipid nanoparticle manufacturing process was used to create formulations of Noopept (N-phenylacetyl-L-prolyglygice ethyl ester). Noopept and lipids were dissolved in ethanol at elevated temperature and dried to form a film. Films were backed filled with dry nitrogen gas and stored at 4° C. for 12-24 hours before proceeding. Films were hydrated with warm water and mixed for 30 minutes before microfluidization, the final formulation volume was 100 mL. Formulations studied in this example are summarized in the table below.

TABLE 19

| Ingredient | Formulation 1 | | Formulation 2 | | Formulation 3 | | Formulation 4 | | Formulation 5 | |
|---|---|---|---|---|---|---|---|---|---|---|
| H100-3 PC | 5 | grams | 5 | grams | 5 | grams | 5 | grams | 5 | grams |
| Cholesterol | 0.5 | grams | 0.5 | grams | 0.5 | grams | 0.5 | grams | 0.5 | grams |
| MCT | 4.8 | grams | 4.8 | grams | 0.28 | grams | 4.8 | grams | 0.28 | grams |
| Noopept | 0 | grams | 1 | gram | 1 | gram | 2 | grams | 2 | grams |
| Vitamin E | 0.05 | grams | 0.05 | grams | 0.05 | grams | 0.05 | grams | 0.05 | grams |
| Purified | QS 100 mL | | QS 100 mL | | QS 100 mL | | QS 100 mL | | QS 100 mL | |
| Water | | | | | | | | | | |

Formulations 1-5 were placed on a 90 day stability study at 2-8° C., 25° C. with 60% relative humidity, and 40° C. with 75% relative humidity. The initial particle size measurements and measurements after 90 days at each stability temperature are shown in the table below.

TABLE 20

| | | Initial | | 90 Days At | 90 Days At |
|---|---|---|---|---|---|
| | | Measure- | 90 Days at | 25° C./ | 40° C./ |
| | | ment | 2-8° C. | 60% RH | 75% RH |
| Formu- | Z-Average | 95.2 nm | 104.8 nm | 95.7 nm | 109.2 nm |
| lation 1 | PDI | 0.153 | 0.162 | 0.153 | 0.249 |
| | D90 | 133.3 nm | 164.7 nm | 135.7 | 277.5 nm |
| Formu- | Z-Average | 104.0 nm | 108.0 nm | 104.9 nm | 195.2 nm |
| lation 2 | PDI | 0.187 | 0.150 | 0.197 | 0.438 |
| | D90 | 187.5 nm | 166.7 nm | 210.3 nm | 774.3 nm |
| Formu- | Z-Average | 141.3 nm | 137.8 nm | 136.6 nm | 756.9 nm |
| lation 3 | PDI | 0.222 | 0.176 | 0.169 | 0.227 |
| | D90 | 396.0 nm | 291.0 nm | 274.3 nm | 1973.3 nm |
| Formu- | Z-Average | 108.4 nm | 111.6 nm | 116.8 nm | 140.8 nm |
| lation 4 | PDI | 0.177 | 0.165 | 0.278 | 0.513 |
| | D90 | 188.7 nm | 199.3 nm | 189.7 nm | 3840.0 nm |
| Formu- | Z-Average | 146.9 nm | 146.7 nm | 150.0 nm | 582.5 nm |
| lation 5 | PDI | 0.172 | 0.148 | 0.171 | 0.297 |
| | D90 | 315.3 nm | 297.3 nm | 336.5 nm | 8090.0 nm |

Long-term stability of the formulation at room temperature and during temperature excursions (i.e. at 40° C. or higher) was improved by drying the Noopept lipid formulation to a powder. This was accomplished by dissolving 5% (w/v) of trehalose into the formulation and lyophilization to a dried cake as outline in Example 4. Fried formulations were broken up with a spatula, milled, followed by sieving through 75 to 34 micrometer sieves to achieve a fine powder. Powders were weighed into vials, back-filled with nitrogen and capped for long-term storage.

The Noopept lipid nanoparticle formulations may be modified further by co-incorporating a cannabinoid, such as CBD, CBG, CBN, or CBDa into the formulation. The formulation may be stored as a liquid or dried to a powder as outlined in Example 4.

### Example 21. Melatonin and CBD Lipid Nanoparticle Formulations

Lipid nanoparticle formulations containing melatonin alone and melatonin and CBD were prepared using the solvent based manufacturing process. Melatonin alone or melatonin and CBD were, along with the other lipid ingredients, partially to completely dissolved in ethanol prior to drying to a film. The film was blanketed in nitrogen gas and stored for a period of 12 to 24 hours at 4° C. prior to processing. Solid lipid films were hydrated with warm water and mixed for 30 minutes to form a lipid slurry before being microfluidized. All formulations were prepared in 100 mL batches. The table below summarizes the formulations made in this example.

TABLE 21

| | H100- | | | Melato- | Vitamin | Choles- | Purified |
|---|---|---|---|---|---|---|---|
| Formu- | 3 PC | MCT | CBD | nin | E | terol | Water |
| lations | (g) | (g) | (g) | (g) | (g) | (g) | (mL) |
| 1 | 10 | 9.5 | 0 | 0 | 0.05 | 1 | QS 100 |
| 2 | 10 | 9.5 | 0 | 0.1 | 0.05 | 1 | QS 100 |
| 3 | 10 | 9.5 | 0 | 1 | 0.05 | 1 | QS 100 |
| 4 | 10 | 9.5 | 0 | 2.5 | 0.05 | 1 | QS 100 |
| 5 | 10 | 9.5 | 0 | 0.5 | 0.05 | 1 | QS 100 |
| 6 | 10 | 9.5 | 2 | 0 | 0.05 | 1 | QS 100 |
| 7 | 10 | 9.5 | 2 | 0.1 | 0.05 | 1 | QS 100 |
| 8 | 10 | 9.5 | 2 | 1 | 0.05 | 1 | QS 100 |
| 9 | 10 | 9.5 | 2 | 2.5 | 0.05 | 1 | QS 100 |
| 10 | 10 | 9.5 | 2 | 0.5 | 0.05 | 1 | QS 100 |

CBD and melatonin lipid nanoparticles were spray dried to a powder after the addition of trehalose to the liquid feed solution. Formulations were spray dried as outlined in Example 4. Prior to forming a powder, the initial particle size distribution was measured for Formulations 1-5 (melatonin only) and summarized in the table below. Powder formulations were sieved successively through 75 to 34 microns. Residual moisture for the powders was measured to be less than 6% for all formulations.

TABLE 22

| | Z-Average | Polydispersity | D90 |
|---|---|---|---|
| | Particle Size | Index | Particle Size |
| Formulation 1 | 100.6 nm | 0.166 | 166.7 nm |
| Formulation 2 | 108.3 nm | 0.186 | 197.3 nm |
| Formulation 3 | 201.8 nm | 0.351 | Not Available |
| Formulation 4 | 156.2 nm | 0.325 | 731.3 nm |
| Formulation 5 | 137.9 nm | 0.235 | 310 nm |

### Example 22. Lipid Nanoparticle Powder Formulations Of CBD, Melatonin, and GABA

The following lipid nanoparticle formulations are designed to promote sleep. The formulations were prepared using the solvent based manufacturing process in 200 mL batches. All lipids, CBD, and melatonin was dissolved in ethanol and dried to a film. The film was hydrated with a warm media containing up to 1.052 mg/mL each of sodium benzoate and potassium sorbate, and up to 0.622 mg/mL each of citric acid monohydrate and malic acid. After processing, GABA (gamma-aminobutyric acid) was dissolved into the lipid nanoparticle suspension and allowed to mix for 2 hours before characterization and spray drying (as outlined above).

TABLE 23

| Ingredient | Formulation 1 | | Formulation 2 | | Formulation 3 | | Formulation 4 | |
|---|---|---|---|---|---|---|---|---|
| H100-3 PC | 20 | grams | 20 | grams | 20 | grams | 20 | grams |
| Cholesterol | 2.0 | grams | 2.0 | grams | 2.0 | grams | 2.0 | grams |
| MCT | 19.0 | grams | 19.0 | grams | 19.0 | grams | 19.0 | grams |
| CBD | 4.0 | grams | 4.0 | grams | 4.0 | grams | 4.0 | grams |
| Melatonin | 400 | mg | 400 | mg | 200 | mg | 200 | mg |
| Vitamin E | 0.1 | grams | 0.1 | grams | 0.1 | grams | 0.1 | grams |
| GABA | 0 | grams | 10 | grams | 0 | grams | 10 | grams |

| | | | | |
|---|---|---|---|---|
| Hydration Media | QS 200 mL | QS 200 mL | QS 200 mL | QS 200 mL |

Initial particle size measurements of the four formulations in liquid form are summarized in the table below. Data shown are the average±standard deviation of three independent measurements.

TABLE 24

| | Formula- | Formula- | Formula- | Formula- |
|---|---|---|---|---|
| Parameter | tion 1 | tion 2 | tion 3 | tion 4 |
| Z-Average | 113.9 ± | 110.5 ± | 103.2 ± | 111.5 ± |
| Particle Size | 1.74 nm | 1.02 nm | 4.68 nm | 1.12 nm |
| Polydispersity | 0.254 ± | 0.186 ± | 0.203 ± | 0.191 ± |
| Index | 0.004 | 0.009 | 0.021 | 0.020 |

### Example 23. Stability of CBD Lipid Nanoparticles in Simulated Gastric and Intestinal Fluids

The stability of CBD Lipid Nanoparticles through the digestive process was simulated by measuring the particle size distribution in simulated gastric fluid after 2 hours, followed by dilution and incubation in simulated intestinal fluid after 4 hours. The CBD lipid nanoparticles were prepared using the solvent based manufacturing process at the 100 mL scale. Simulated gastric fluid was prepared by dissolving/dispersing 1 gram of sodium chloride (CAS 7647-14-5), 21.5 mg of sodium taurocholate (CAS 345909-26-4), 6.5 mg of lecithin (CAS 8002-43-5), and sufficient hydrochloric acid (CAS 7647-01-0) into purified water (QS 500 mL) to achieve a final pH of 1.6. Simulated intestinal fluid was prepared by dissolving/dispersing 1 gram of sodium chloride (CAS 7647-14-5), 806.5 mg of sodium taurocholate (CAS 345909-26-4), 64.4 mg of lecithin (CAS 8002-43-5), 1.1 grams of maleic acid (CAS 110-16-7), and 696 mg of sodium hydroxide (CAS 1310-73-2) in purified water (QS 500 mL). The pH was adjusted to 6.5 as needed. Simulated solutions were used immediately or stored at 4° C. for no longer than 1 month.

Prior to starting the study, simulated gastric and intestinal fluids were equilibrated to 37° C. Spectrum Laboratories Float-A-Lyzer G2 Dialysis devices (50 kD MWCO, 1 mL, Catalog #G235034) were equilibrated in 37° C. water prior to use. The initial particle size distribution was measured before starting the experiment. One mL of CBD lipid nanoparticles was placed on the interior of the Float-A-Lyzer, the cap was affixed, and the Float-A-Lyzer was placed into 20 mL of simulated gastric fluid inside a 50 mL conical tube. The conical tube containing the simulate fluid and sample was placed inside a 37° C. shaker incubator for 2 hours. At the end of the first incubation a sample was taken for particle size analysis. Immediately, the Float-A-Lyzer is placed in a new conical tube containing 20 mL of simulated intestinal fluid and returned to the 37° C. shaker incubator for 4 hours. At the end of the second incubation a sample was taken for particle size analysis. The total time of the experiment was 6 hours. Three commercially available, oil-based CBD products were analyzed similarly. All samples were measured in triplicate.

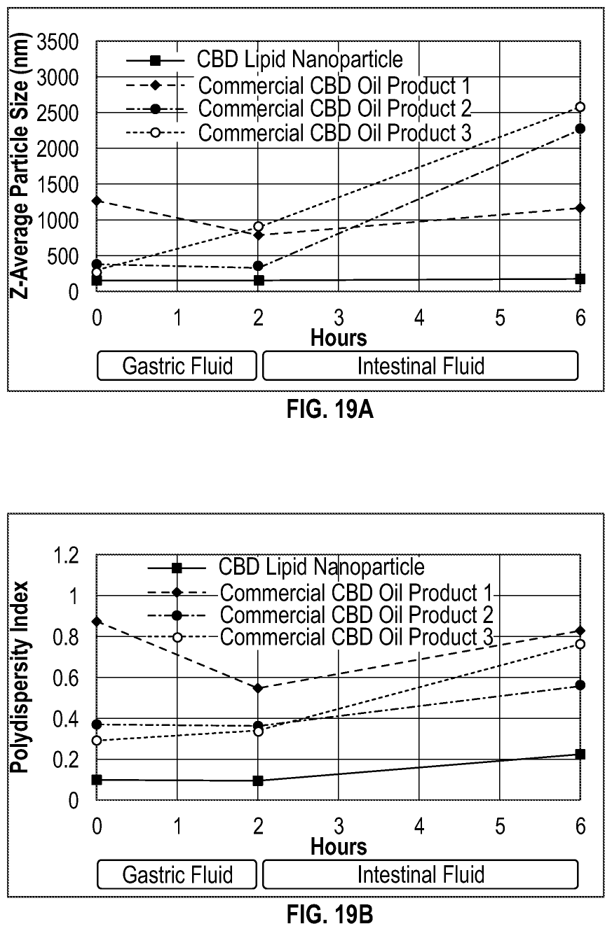

Shown in FIGS. 19A and 19B show the change in particle size and polydispersity index over the incubation period in simulated gastric and intestinal fluid. The CBD lipid nanoparticles experienced no change in particle size and a modest increase of PDI during the full incubation period. All the commercial oil-based CBD products experienced fluctuations in particle size and PDI during the incubation in simulated gastric and/or intestinal fluids, indicating an instability in the formulation during the digestive process.

### Example 24. Preparation of CBD Lipid Nanoparticles with Oil Based, Less Pure Phospholipids

CBD containing lipid nanoparticles were prepared using the solvent based manufacturing process in 0.1 liter batches. Lipid nanoparticles were prepared with oil based phospholipids and compared to the 99.0% pure phosphatidylcholine (H100-3). The compositions of the oil based phospholipids are provided in the table below under composition (information taken from manufacturer's COA), along with the initial particle size distribution measurements.

All formulations were prepared with 10% w/w phospholipid (Ingredient shown in the table below), 2% w/w CBD, 9.5% w/w medium chain triglycerides, 0.1% w/w vitamin E, and between 77.4 and 78.4% w/w purified water. The sample prepared with H100-3 phospholipid also had 1% w/w of cholesterol added.

TABLE 25

| | | Initial | | Initial |
|---|---|---|---|---|
| | | Z-Ave | | D90 |
| Ingre- | | Particle | Initial | Particle |
| dient | Composition | Size | PDI | Size |
| Alcolec | Saturated fatty acids: ~35% | 187.1 ± | 0.090 ± | 326.3 ± |
| E 20 O | Monounsaturated fatty | 1.31 | 0.011 | 7.64 |
| (American | acids: ~36% | nm | | nm |
| Lecithin | Polyunsaturated fatty acids | | | |
| Company) | (C18:2, C18:3): ~19% | | | |
| | Arachidonic acid | | | |
| | (C20:4): ~2.5% | | | |
| | Docosahexaenoic acid | | | |
| | (C22:6): ~2% | | | |
| | Cholesterol: Trace | | | |
| Alcolec | Saturated fatty acids: 26-32% | 213.9 ± | 0.100 ± | 396.3 ± |
| E 80 O | Monounsaturated fatty | 1.76 | 0.016 | 17.90 |
| (American | acids: 17-19% | nm | | nm |
| Lecithin | Polyunsaturated fatty acids | | | |
| Company) | (C18:2, C18:3): 8-12% | | | |
| | Arachidonic acid (C20:4): 3-5% | | | |
| | Docosahexaenoic acid | | | |
| | (C22:6): 1.5-2.5% | | | |
| | Cholesterol: 1.9-6.6% | | | |
| H100-3 | Saturated fatty acids: 99.0% | 91.9 ± | 0.119 ± | 143.3 ± |
| (American | Monounsaturated and | 0.61 | 0.018 | 11.85 |
| Lecithin | polyunsaturated fatty | nm | | nm |
| Company) | acids: 0.4% | | | |
| | Cholesterol: not detected | | | |

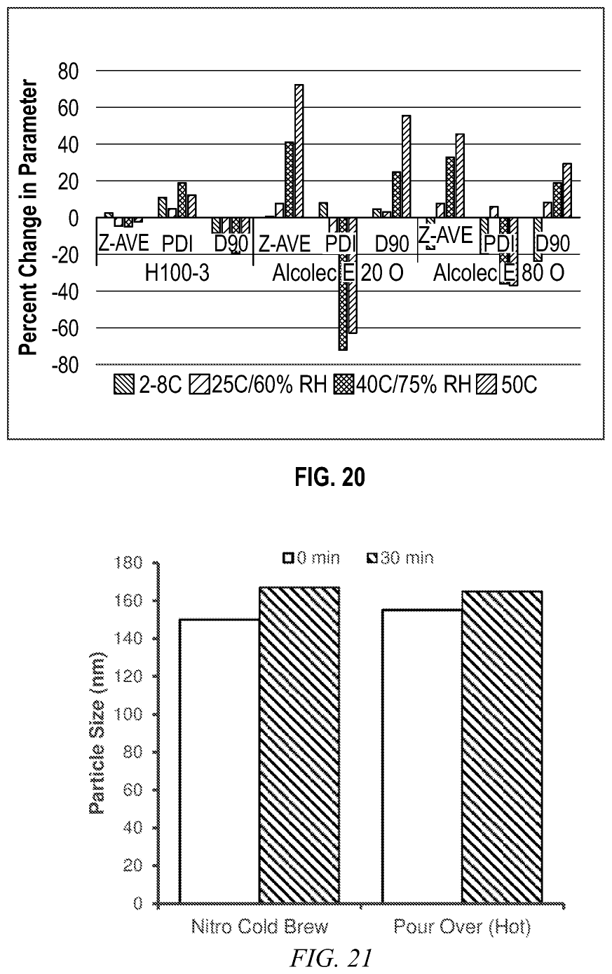

Samples were placed at four storage conditions for a preliminary, short-term 2-week stability experiment. At the end of the incubation period, samples were measure for particle size distribution and percent changes were examined. For the sample prepared with H100-3 phospholipid, no parameter changed more than 20% from its initial measurement at any storage condition, indicating a stable product. Samples prepared with the less pure, oil-based phospholipids experienced significant changes in particle size parameters over the 2 week incubation period in one or more of the storage conditions, indicating a less stable product compared to lipid nanoparticles prepared with H100-3 phospholipid. Results are shown in FIG. 20.

### Example 25. Examples of Sweeteners

CBD lipid nanoparticles were prepared using the solvent based manufacturing process at the 100 mL batch size. Dried lipid films were hydrated with a hydration media containing up to 1.052 mg/mL each of sodium benzoate and potassium sorbate, and up to 0.622 mg/mL each of citric acid monohydrate and malic acid as preservatives. A sweetener (0.09% w/w) was dissolved in the hydration media prior to adding to the dried film based on the formulations table below, no additional flavoring agent was added to the formulation. A day after processing the formulations were screened for initial particle size distribution (shown in the table below). Initial particle size measurements indicate that all sweeteners evaluated from Monkfruit Corporation, GLG Corporation, and Tate and Lyle are compatible with the CBD lipid nanoparticle formulation.

TABLE 26

| | | Initial | | |
|---|---|---|---|---|
| | | Z-Average | | Initial |
| | | Particle | | Polydispersity |
| Formulation | Sweetener | Size | | Index |
| Formulation 1 | Monk Fruit Corporation | 106.0 | nm | 0.209 |
| | Catalog: MFC-J3.5 | | | |
| | 3.5% Mogrosides | | | |
| Formulation 2 | Monk Fruit Corporation | 99.0 | nm | 0.158 |
| | Catalog: MFC-E30P | | | |
| | 30% Mogrosides, de- | | | |
| | proteined | | | |
| Formulation 3 | Monk Fruit Corporation | 106.0 | nm | 0208 |
| | Catalog: MFC-E50 | | | |
| | 50% Mogrosides | | | |
| Formulation 4 | Monk Fruit Corporation | 108.0 | nm | 0.197 |
| | Catalog: MFC-E55 | | | |
| Formulation 5 | Monk Fruit Corporation | 106.0 | nm | 0.186 |
| | Catalog: MFC-E80 | | | |
| | 80% Mogrosides | | | |
| Formulation 6 | GLG Life Tech Corporation | 107.0 | nm | 0.214 |
| | Catalog: GLG-MV55 | | | |
| | 55% Mogrosides | | | |
| Formulation 7 | GLG Life Tech Corporation | 97.0 | nm | 0.191 |
| | Catalog: GLG-RA97 | | | |
| | 97% Rebaudioside A | | | |
| Formulation 8 | Tate and Lyle | 94.0 | nm | 0.183 |
| | Catalog: TL-Stevia 3.05 | | | |
| | 95% Steviol Glycosides | | | |
| Formulation 9 | Tate and Lyle | 100.0 | nm | 0.218 |
| | Catalog: TL-Stevia 3.10 | | | |
| | 95% Steviol Glycosides | | | |
| Formulation 10 | No Sweetener | 98.0 | nm | 0.148 |

### Example 26: Comparator Products

CBD comparator products with a common ingredient or label were purchased from the original manufacturer's website for particle size comparison to the embodiments described within. A key ingredient used in this search was phosphatidylcholine, phospholipids, lecithin, or MCT. Key words found on the label include nano, liposomal, and water soluble. Products were diluted into filtered, ultra-pure water to an optical density that yielded a suitable count for particle size measurement. The table below summarizes the particle sizes measured from the comparator products. All products measured had a particle size and polydispersity index that exceeds the formulations described herein, further supporting that the choice of ingredients and manufacturing process are key to producing a stable, nanoparticle.

TABLE 27

| | Label | Label | Z-Average | | Polydispersity | D90 | |
|---|---|---|---|---|---|---|---|
| Comp. | Ingredients | Claim | Particle Size | | Index | Particle Size | |
| 1 | Full Hemp Extract, | Liposomal | 1,050.0 | nm | 0.350 | Not | |
| | Phospholipids with 50% | CBD | | | | Available | |
| | phosphatidylcholine | | | | | | |
| | (organic lecithin), | | | | | | |
| | Water, Xylitol, | | | | | | |
| | Glycerol, Sorbic | | | | | | |
| | Acid, Vitamin E, | | | | | | |
| | Pineapple Flavoring | | | | | | |
| 2 | Purified Water, | NanoCBD | 790.0 | nm | 0.310 | Not | |
| | Olive Oil, | Suspended | | | | Available | |
| | Sunflower Lecithin, | in Water | | | | | |
| | Anhydrous Hemp | | | | | | |
| | Oil, Potassium | | | | | | |
| | Sorbate, Vitamin E, | | | | | | |
| | Citric Acid | | | | | | |
| 3 | Ultra-pure Nano | CBD | 7,493.0 | nm | 1.000 | >10,000.0 | nm |
| | Water, MCT Oil, | Water | | | | | |
| | Natural Gums, | Soluble | | | | | |
| | Vegetable Glycerin, | Liquid | | | | | |
| | Citric Acid, | 25 nm | | | | | |
| | Potassium Sorbate, | Particle | | | | | |
| | Sodium Benzoate | Size | | | | | |
| 4 | Purified Water, | Nano CBD | 243.4 | nm | 0.428 | 4970.0 | nm |
| | Hemp Extract, | | | | | | |
| | Saponin Extract, | | | | | | |
| | Ascorbic Acid | | | | | | |
| 5 | Vegetable Glycerin, | Water | 929.7 | nm | 0.583 | 8,970.0 | nm |
| | CBD Extract, | Soluble | | | | | |
| | Hydrosome | Rapid | | | | | |
| | Electrolyte Blend, | Release | | | | | |
| | Polysorbate 80 | | | | | | |
| 6 | Organic Vegetable | Fast Acting | 7,140.0 | nm | 1.000 | >10,000.0 | nm |
| | Glycerin, Water, | Water | | | | | |
| | Quillaja Extract, | Soluble | | | | | |
| | CBD Hemp Oil, | | | | | | |
| | Moringa, Acerola | | | | | | |
| | Cherry, Vitamin C+ | | | | | | |
| 7 | Hemp Seed Oil, | Tincture | 2,408.0 | nm | 1.000 | 9,930.0 | nm |
| | CBD Extract | with | | | | | |
| | | BioPrime | | | | | |
| | | Nanoparticle | | | | | |
| | | Delivery | | | | | |
| | | Technology | | | | | |
| 8 | Hemp-Derived | Nanoliposo | 884.5 | nm | 0.714 | >10,000.0 | nm |
| | CBD, Sunflower | mal CBD | | | | | |
| | Lecithin, Cellulose, | Powder | | | | | |
| | Calcium Phosphate | | | | | | |

### Example 27 CBD Lipid Nanoparticle Topical Lotion

Shown in the table below is a topical formulation utilizing the CBD lipid nanoparticle system as a carrier for CBD in a lotion/cream for surface pain relief. The base of the formulation (Phase A) utilizes Lipoid's Skin Lipid Matrix 2026 technology and is present in the final formulation at 50%. The CBD (50 mg/mL) lipid nanoparticle (Phase B) composition is described in other embodiments, but here without preservatives and flavoring, and is present in the final formulation at 20% (1% CBD). Phase C of the composition consists of lipid/oil based ingredients or oil soluble ingredients, and includes Captex 170 EP as a skin permeation enhancer, argan oil, menthol, arnica oil, camphor, and grapefruit seed oil present in total at 19% in the final formulation. Where menthol, arnica oil, camphor, and grapefruit seed oil are present for their topical analgesic properties. Lastly, Phase D of the composition is water and is present at 11%.

The lotion ingredients were combined through cold mixing. First, all ingredients in Phase C were combined and mixed until dissolved. Phase B was manufactured according to solvent based method described in previous embodiments. Phase A was combined with Phase A and mixed with a planetary mixer for 2 minutes at 2000 RPM. Phase C was added 5 mL at a time, followed by hand mixing with a spatula. When all of Phase C was added, the composition was further mixed for 2 minutes at 2000 RPM in a planetary mixer. Phase B was added 5 mL at a time, followed by hand mixing with a spatula. When all of Phase B was added, the composition was mixed a final time for 2 minutes at 2000 RPM in a planetary mixer. The batch of lotion was 100 mL and contained 1% of CBD.

Additional lotions were prepared with other permeation enhancers. For example, 5% of Captex 170 EP was replaced with 5% of dimethyl sulfoxide or 5% of dimethyl isosorbide. Additional lotions were prepared with additional topical analgesics such as lidocaine, wintergreen oil, or terpenes such as guaiacol.

TABLE 28

| Phase | Ingredient | INCI | Function | Supplier | % w/w |
|---|---|---|---|---|---|
| A | SLM2026 | Water, Caprylic/Capric | Base | Lipoid | 50% |
| | | Triglyceride, Hydrogenated | Formulation | | |
| | | Phosphatidylcholine, | | | |
| | | Pentylene Glycol, Glycerin, | | | |
| | | Butyrospermum Parkil | | | |
| | | Butter, Squalene, Ceramide | | | |
| | | NP | | | |
| | | CBD (50 mg/mL), | | | |
| B | CBD Lipid | phosphatidylcholine, | CBD carrier | | 20% |
| | Nanoparticles | medium chain triglycerides, | | | |
| | | cholesterol, vitamin E | | | |
| C | Captex 170 EP | Caprylic/Capric Acid Ester | Permeation | ABITEC | 5% |
| | | of Saturated Fatty Alcohol | Enhancer | | |
| | | C12-C18 | | | |
| | Argan Oil | Argania Spinosa Kernel Oil | Topical | Varies | 5% |
| | Menthol | Menthol | Analgesics | | 5% |
| | Arnica Oil | Arnica Montana Extract | | | 3% |
| | Camphor | Camphor | | | 0.5% |
| | Grapefruit Oil | Citrus Paradisi Seed Oil | | | 0.5% |
| D | Deionized | Water | Diluent | | 11% |
| | Water | | | | |

### Example 28: Stability of CBD Lipid Nanoparticles in Hot and Cold Coffee Products

CBD lipid nanoparticles was dispersed in coffee beverages at a concentration of 10 mg CBD per 8 ounce coffee beverage. A hot coffee beverage was prepared using the pour over technique, the resulting liquid was 130° F. at the time the CBD lipid nanoparticles were introduced. CBD nanoparticles were also dispersed in a nitro cold brew coffee (Parks Coffee), the coffee beverage was at 2-8° C. at the time the nanoparticles were introduced. After 30 minutes of storage in the beverage, the coffee was diluted for particle size measurement. The initial particle size measurement in each solution was compared to the particle size after 30 minutes of storage in two coffee beverages. As shown in the FIG. 21, the particle size only increased by 11.3% and 6.5% in cold and hot coffee beverages over 30 minutes, respectively, indicating the CBD lipid nanoparticles are stable in coffee beverages.

### Example 29: Viscosity Measurement of an Embodiment

The viscosity of the CBD lipid nanoparticles (as prepared above in Example 1) was measured using a low volume adapter attached to a LV-DV-II+Brookfield viscometer (Brookfield, Middleboro, Mass.). The viscosity was determined using 16 mL of solution at 26° C. and a spindle speed of 60 RPM, measured over 3 minutes. The viscosity of the CBD lipid nanoparticle solution was determined to be 5.096 Cp.

### Example 30: Preparing Lipid Nanoparticles Containing Synthetic CBD

A composition for the delivery of synthetic CBD was prepared using the methods similar to those disclosed in Example 1. Particle size and zeta potential of liquid was measured on a Malvern ZS90 Zetasizer (Malvern, UK). The liquid product was diluted at least 50 times in purified water and the equivalent of 1 mg of CBD in a powder form was dissolved in 1 mL of purified water for measurements. Cannabinoids concentration was measured by ultra high-pressure liquid chromatography (UHPLC). The mean particle size was found to be 97.4 nm with a D90 of 146.7 nm. The PDI was 0.168 and the nanoparticles had a zeta potential of +4.2 mV. The synthetic CBD concentration was measured to be at 1.96%. Results are summarized in the table below.

TABLE 29

| | | Synthetic CBD |
|---|---|---|
| Parameter | Specification | Nanoparticle |
| Z-Average Particle Size | 20-500 nm | 97.4 nm |
| Polydispersity Index | Report Only | 0.168 |
| D90 Particle Size | Report Only | 146.7 nm |
| Zeta Potential | Report Only | +4.2 mV |
| CBD Concentration | 1.8%-2.2% | 1.96% |

### Example 31: Preparing Lipid Nanoparticles Containing Synthetic CBDV

A composition for the delivery of synthetic CBDV was prepared using the methods similar to those disclosed in Example 1. The resulting particle size and zeta potential were determined using a Malvern ZetaSizer and CBDV concentration was determined using a UPLC. The mean particle size was found to be 92.4=1 nm with a D90 of 127.7 nm. The PDI was 0.140 and the nanoparticles had a zeta potential of +5.04 mV. The synthetic CBDV concentration was measured to be at 1.801%. Results are summarized in the table below.

TABLE 30

| | | Synthetic CBDV |
|---|---|---|
| Parameter | Specification | Nanoparticle |
| Z-Average Particle Size | 20-500 nm | 92.1 nm |
| Polydispersity Index | Report Only | 0.140 |
| D90 Particle Size | Report Only | 127.7 nm |
| Zeta Potential | Report Only | +5.04 mV |
| CBDV Concentration | 1.62%-1.98% | 1.801% |

### Example 32: Preparing Lipid Nanoparticles Containing Synthetic CBG

A composition for the delivery of synthetic CBG was prepared using methods similar to those disclosed in Example 1. The resulting particle size and zeta potential were determined using a Malvern ZetaSizer and CBG concentration was determined using a UPLC. The mean particle size was found to be 95.7 nm with a D90 of 132.0 nm. The PDI was 0.103 and the nanoparticles had a zeta potential of +5.08 mV. The synthetic CBG concentration was measured to be at 1.664%. Results are summarized in the table below.

TABLE 31

| | | Synthetic CBG |
|---|---|---|
| Parameter | Specification | Nanoparticle |
| Z-Average Particle Size | 20-500 nm | 95.7 nm |
| Polydispersity Index | Report Only | 0.103 |
| D90 Particle Size | Report Only | 132.0 nm |
| Zeta Potential | Report Only | +5.08 mV |
| CBG Concentration | 1.44%-1.76% | 1.664% |

### Example 33: Preparing Lipid Nanoparticles Containing CBT Distillate

A composition for the delivery of CBT distillate was prepared using methods similar to those in Example 1. The resulting particle size and zeta potential were determined using a Malvern ZetaSizer and CBT concentration was not determined. The mean particle size was found to be 80.4 nm with a D90 of 104.3 nm. The PDI was 0.144 and the nanoparticles had a zeta potential of +6.81 mV. Results are summarized in the table below.

TABLE 32

| | | Synthetic CBT |
|---|---|---|
| Parameter | Specification | Nanoparticle |
| Z-Average Particle Size | 20-500 nm | 80.4 nm |
| Polydispersity Index | Report Only | 0.144 |
| D90 Particle Size | Report Only | 104.3 nm |
| Zeta Potential | Report Only | +6.81 mV |

### Example 34: Preparing Lipid Nanoparticles Containing Cannabinoids and Alpha-Pinene

A composition for the delivery of hemp distillate with alpha-pinene terpene added was prepared using procedures similar to Example 1. The hemp distillate was determined to contain CBD, CBG, CBN, CBC, and CBDV. When all ingredients were dissolved, the solvent was removed to form a dried composition. The resulting particle size and zeta potential were determined using a Malvern ZetaSizer and cannabinoid concentrations were determined by UPLC. Terpene concentrations were determined by GC-FID.

After the formulation was diluted to a theoretical CBD concentration of 12 mg/mL, the cannabinoid profile was measured by UPLC and results shown in the table below. The CBD concentration was found to 1.229% and minor cannabinoids of CBG, CBN, CBC, and CBDV were also detectable. Being a hemp distillate, terpenes were detectable in the formulation; however, the predominant terpene was alpha-pinene, the terpene intentionally added to the formulation. The mean particle size of the formulation was found to be 86.5 nm with a D90 of 114.0 nm. The PDI was 0.082 and the nanoparticles had a zeta potential of +4.02 mV.

TABLE 33

| | Measured | Measured |
|---|---|---|
| Cannabinoid | Concentration (w/w %) | Concentration (mg/g) |
| CBD | 1.229% | 12.289 mg/g |
| CBDa | <LOQ | <LOQ |
| Δ9-THC | <LOQ | <LOQ |
| Δ8-THC | <LOQ | <LOQ |
| Exo-THC | <LOQ | <LOQ |
| THCa | <LOQ | <LOQ |
| CBG | 0.042% | 0.419 mg/g |
| CBGa | <LOQ | <LOQ |
| CBN | 0.042% | 0.419 mg/g |
| CBNa | <LOQ | <LOQ |
| CBC | 0.032 | 0.323 mg/g |
| CBCa | <LOQ | <LOQ |
| CBL | <LOQ | <LOQ |
| CBDV | 0.004% | 0.040 mg/g |
| CBDVa | <LOQ | <LOQ |
| THCV | <LOQ | <LOQ |
| THCVa | <LOQ | <LOQ |

TABLE 34

| | Measured | Measured |
|---|---|---|
| Terpene | Concentration (w/w %) | Concentration (mg/g) |
| alpha-Pinene | 0.521% | 5.205 mg/g |
| Camphene | 0.005% | 0.046 mg/g |
| beta-Myrecene | <LOQ | <LOQ |
| beta-Pinene | 0.003% | 0.026 mg/g |
| Carene | <LOQ | <LOQ |
| e-Ocimene | <LOQ | <LOQ |
| delta-Limonene | <LOQ | <LOQ |
| p-Cymene | <LOQ | <LOQ |
| z-Ocimene | <LOQ | <LOQ |
| Eucalyptol | <LOQ | <LOQ |
| y-Terpinene | <LOQ | <LOQ |
| Terpinolene | <LOQ | <LOQ |
| Linalool | <LOQ | <LOQ |
| Isopulegol | <LOQ | <LOQ |
| Geraniol | <LOQ | <LOQ |
| Beta-Caryophyllene | 0.003% | 0.026 mg/g |
| Humulene | <LOQ | <LOQ |
| cis-Nerolidol | <LOQ | <LOQ |
| trans-Nerolidol | <LOQ | <LOQ |
| alpha-Guaiol | 0.002% | 0.023 mg/g |
| Caryophyllene Oxide | <LOQ | <LOQ |
| alpha-Bisabolol | 0.006% | 0.062 mg/g |

TABLE 35

| | | Hemp Distillate |
|---|---|---|
| Parameter | Specification | Nanoparticle |
| Z-Average Particle Size | 20-500 nm | 86.5 nm |
| Polydispersity Index | Report Only | 0.082 |
| D90 Particle Size | Report Only | 114.0 nm |
| Zeta Potential | Report Only | +4.02 mV |

### Example 35: Compositions That Survive Pasteurization

Lipid nanoparticles containing 4% CBD (40 mg/mL) were prepared by forming an oil-in-water emulsion using methods similar to those disclosed in Example 1.

To replicate pasteurization in a manufacturing setting (i.e. beverage manufacturing), 200 mL of deionized water was heated to a critical temperature and allowed to stabilize. After stabilization, encapsulated CBD was introduced to the water such that the CBD was at a final concentration of 0.1 mg/mL. Depending on the temperature, the diluted encapsulated CBD was held for the specific time to achieve pasteurization. After the required time has lapsed, the solution was cooled to room temperature (20-25° C.). The following pasteurization conditions were evaluated.
1. Critical temperature of 89° C. for a 1 second hold time.
2. Critical temperature of 72° C. for a 15 second hold time.
3. Critical temperature of 63° C. for a 30 minute hold time.

A non-pasteurized solution was prepared as a control. A sample of the solution was collected after each condition cooled to room temperature and again after 1 week of storage at room temperature. Samples were evaluated for CBD concentration by UPLC and particle size analysis by dynamic light scattering (Z-average). No significant differences were detected between pasteurization conditions or 1 week after pasteurization with CBD concentration, particle size increased slightly after pasteurization.

TABLE 36

| Parameter | Condition 1 | | Condition 2 | | Condition 3 | | Non-Pasteurized | |
|---|---|---|---|---|---|---|---|---|
| Initial CBD | 0.011 | w/w % | 0.011 | w/w % | 0.011 | w/w % | 0.010 | w/w % |
| Concentration | 0.109 | mg/g | 0.106 | mg/g | 0.107 | mg/g | 0.101 | mg/g |
| 1-week CBD | 0.011 | w/w % | 0.010 | w/w % | 0.011 | w/w % | 0.010 | w/w % |
| Concentration | 0.107 | mg/g | 0.104 | mg/g | 0.109 | mg/g | 0.104 | mg/g |
| Initial Particle Size | 96.84 | nm | 95.72 | nm | 95.11 | nm | 96.98 | nm |
| 1-week Particle Size | 120.8 | nm | 121.3 | nm | 122.0 | nm | 97.92 | nm |

### Example 36: Compositions That Survive Ozonation

Lipid nanoparticles containing 4% CBD (40 mg/mL) were prepared by forming an oil-in-water emulsion using methods similar to those disclosed in Example 1.

To replicate the ozonation process in a manufacturing setting (i.e. beverage manufacturing), encapsulated CBD was diluted to a final CBD concentration of 0.2 mg/mL in 500 mL of deionized water. An Ozone Air & Water Purifier was used to ozonate the encapsulated CBD solution for up to 30 minutes. At 10-minute intervals (up to 30 minutes) a sample of the solution was collected for CBD concentration by UPLC and particle size analysis (Z-average) by dynamic light scattering. Ozonated solutions were stored at room temperature for 1 week and analyzed again for CBD concentration and particle size analysis. A non-ozonated solution was prepared as a control. No significant differences were detected between ozonation conditions or 1 week after ozonation with CBD concentration, particle size increased modestly after ozonation.

TABLE 37

| Parameter | 10 Minutes | | 20 Minutes | | 30 Minutes | | Non-Ozonated | |
|---|---|---|---|---|---|---|---|---|
| Initial CBD | 0.019 | w/w % | 0.020 | w/w % | 0.019 | w/w % | 0.022 | w/w % |
| Concentration | 0.191 | mg/g | 0.201 | mg/g | 0.193 | mg/g | 0.216 | mg/g |
| 1-week CBD | 0.019 | w/w % | 0.020 | w/w % | 0.021 | w/w % | 0.020 | w/w % |
| Concentration | 0.197 | mg/g | 0.199 | mg/g | 0.211 | mg/g | 0.202 | mg/g |
| Initial Particle Size | 89.63 | nm | 89.12 | nm | 88.13 | nm | 89.43 | nm |
| 1-week Particle Size | 143.1 | nm | 141.2 | nm | 140.4 | nm | 97.38 | nm |

### Example 37: Compositions That Survive Ultraviolet (UV) Treatment

Lipid nanoparticles containing 4% CBD (40 mg/mL) were prepared by forming an oil-in-water emulsion using methods similar to those disclosed in Example 1.

To replicate the UV treatment process in a manufacturing setting (i.e. beverage manufacturing), encapsulated CBD was diluted to a final CBD concentration of 0.2 mg/mL in 500 mL of deionized water. A UV source was used to UV treat 0.5 liters of encapsulated CBD solution for either 1 cycle or 10 cycles of treatment with mechanical stirring. After each cycle of treatment a sample of the solution was collected for CBD concentration by UPLC. UV treated solutions were stored at room temperature for 1 week and analyzed again for CBD concentration. A non-UV treated sample was collected as a control. No significant differences were detected between UV treatment cycles or 1 week after UV treatment with CBD concentration.

TABLE 38

| Parameter | 1 Cycle | | 10 Cycles | | No UV Treatment | |
|---|---|---|---|---|---|---|
| Initial CBD | 0.022 | w/w % | 0.022 | w/w % | 0.021 | w/w % |
| Concentration | 0.224 | mg/g | 0.215 | mg/g | 0.210 | mg/g |
| 1-week CBD | 0.022 | w/w % | 0.021 | w/w % | 0.021 | w/w % |
| Concentration | 0.221 | mg/g | 0.208 | mg/g | 0.213 | mg/g |

### Example 38: Preparing a Two Ounce Ready-To-Drink (RTD) Beverage For Immunity Support

Lipid nanoparticles containing 2% CBD (20 mg/mL) were prepared by forming an oil-in-water emulsion using methods similar to those disclosed in Example 1.

The 2 ounce RTD beverage for immunity support was constructed by dissolving 125 mcg vitamin D3 (as cholecalciferol), 25 mg of magnesium (as magnesium chloride), and 20 mg of zinc (as zinc gluconate) into 58 mL of deionized water. One mL of the 2% encapsulated CBD solution was added to bring the total volume to 59 mL and CBD concentration to 0.339 mg/mL. Monk Fruit extract and flavor are added as needed.

### Example 39: Preparing a Two Ounce Ready-To-Drink (RTD) Beverage To Promote Calm Sensation

Lipid nanoparticles containing 2% CBD (20 mg/mL) were prepared by forming an oil-in-water emulsion using methods similar to those disclosed in Example 1.

The 2 ounce RTD beverage for calm sensation was constructed by dissolving 200 mg L-Theanine into 57.5 mL of deionized water. One and a half mL of the 2% encapsulated CBD solution was added to bring the total volume to 59 mL and CBD concentration to 0.508 mg/mL. Monk Fruit extract and flavor are added as needed.

### Example 40: Preparing a Two Ounce Ready-To-Drink (RTD) Beverage For Sleep Support

Lipid nanoparticles containing 2% CBN (20 mg/mL) were prepared by forming an oil-in-water emulsion using methods similar to those disclosed in Example 1.

The 2 ounce RTD beverage for sleep support was constructed by dissolving 150 mg gamma-aminobutyric acid (GABA and 100 mg of 5-hydroxytryptophan into 58 mL of deionized water. One mL of the 2% encapsulated CBD solution was added to bring the total volume to 59 mL and CBD concentration to 0.339 mg/mL. Monk Fruit extract and flavor are added as needed.

### Example 41: Preparing a Two Ounce Ready-To-Drink (RTD) Beverage For Energy Support

Lipid nanoparticles containing 2% CBC (20 mg/mL) and 0.5% THCV (5 mg/mL) were prepared by forming an oil-in-water emulsion using methods similar to those disclosed in Example 1.

The 2 ounce RTD beverage for immunity support was constructed by dissolving 40 mg niacin, 4 mg of vitamin B6, 6 mcg of vitamin B12, and 100 mg of L-tyrosine into 58 mL of deionized water. One mL of the 2% encapsulated CBC/0.5% THCV solution was added to bring the total volume to 59 mL and CBC concentration to 0.339 mg/mL and 0.085 mg/mL THCV. Monk Fruit extract and flavor are added as needed.

### Example 42: Method of Treating Anxiety

Based on the inventor's experience, the following prophetic results are projected using controlled studies.

Three groups of patients of age between 45 and 55 are admitted to treatment after having had been diagnosed with anxiety. The first group is treated with a CBD containing lipid-based particle composition as disclosed herein orally. The second group of patients is treated orally with a CBD oil based composition orally. The third group of patients is treated with a placebo orally. The first group of patients experiences recovery from each of the symptoms of anxiety faster than the second group and to a higher degree as measured by a self-evaluation. The patients in the first group report less feelings of nervousness, less feelings of restlessness, less feelings of impending danger, panic or doom, less trouble concentrating, less trouble sleeping. After oral ingestion, the patients in the first group have lower heart rates and less trembling than those in the second group. The results show statistically significant improvements in the first group relative to either the second group or the third group.

The patients in the second group show statistically significant improvement over the placebo, but not to the degree achieved reported by the first group. The patients in the second group have statistically higher reports of side effects associated with treatment than either the first or the third group.

### Example 43: Method of Treating Pain

Based on the inventor's experience, the following prophetic results are projected using controlled studies.

Three groups of female and male patients of age between 25 and 40 are admitted to treatment after having had been diagnosed with pain due to exercise related injuries. The first group is treated with a CBD containing lipid-based particle composition as disclosed herein topically. The second group of patients is treated topically with a competitor liposomal CBD based composition made with CBD oil. The third group of patients is treated with a placebo topically. The first group of patients experiences recovery from pain faster than the second group and to a higher degree as measured by a self-evaluation. The results show statistically significant improvements in the first group relative to either the second group or the third group.

The patients in the second group show statistically significant improvement over the placebo, but not to the degree achieved reported by the first group. The patients in the second group have statistically higher reports of side effects associated with treatment than either the first or the third group.

### Example 44: Method of Treating Premenstrual Syndrome

Based on the inventor's experience, the following prophetic results are projected using controlled studies.

Three groups of women patients of age between 35 and 40 are admitted to treatment after having had been diagnosed with premenstrual syndrome (PMS). The first group is treated with a GABA containing lipid-based particle composition as disclosed herein orally. The second group of patients is treated orally with a competitor liposomal GABA based composition. The third group of patients is treated with a placebo orally. The first group of patients experiences recovery from each of the symptoms of PMS faster than the second group and to a higher degree as measured by a self-evaluation. The patients in the first group report less cramping and less severity of cramping. After oral ingestion, the patients in the first group report having an improved moods. The results show statistically significant improvements in the first group relative to either the second group or the third group.

The patients in the second group show statistically significant improvement over the placebo, but not to the degree achieved reported by the first group. The patients in the second group have statistically higher reports of side effects associated with treatment than either the first or the third group.

### Example 45: Method of Treating Insomnia

Based on the inventor's experience, the following prophetic results are projected using controlled studies.

Three groups of patients of age between 35 and 40 are treated for insomnia. The first group is treated with a GABA/CBD containing lipid-based particle composition as disclosed herein orally. The second group of patients is treated orally with a competitor liposomal GABA/CBD oil based composition. The third group of patients is treated with a placebo orally. The first group of patients experiences faster sleep time than the second group that is statistically significant. The patients in the second group show statistically significant improvement over the placebo, but not to the degree achieved reported by the first group. The patients in the second group have statistically higher reports of side effects associated with treatment than either the first or the second group.

### Example 46: Method of Treating Anxiety

Based on the inventor's experience, the following prophetic results are projected using controlled studies.

Four groups of patients of age between 45 and 55 are admitted to treatment after having had been diagnosed with anxiety. The first group is treated with a CBD and alpha-terpineol containing lipid-based particle composition as disclosed herein orally. The second group is treated with an alpha-terpineol and phytol containing lipid-based particle composition as disclosed herein orally. The third group of patients is treated orally with a CBD oil-based composition orally. The fourth group of patients is treated with a placebo orally. The first and second groups of patients experience recovery from each of the symptoms of anxiety faster than the third group and to a higher degree as measured by a self-evaluation. The patients in the first and second groups report less feelings of nervousness, less feelings of restlessness, less feelings of impending danger, panic or doom, less trouble concentrating, less trouble sleeping. After oral ingestion, the patients in the first and second groups have lower heart rates and less trembling than those in the third group. The first and second group of patients experience lower systemic levels of the hormone cortisol compared to measurements prior to oral treatment, and compared to patients in groups three and four. The results show statistically significant improvements in the first and second groups relative to either the third group or the fourth group.

The patients in the third group show statistically significant improvement over the placebo, but not to the degree achieved reported by the first and second groups. The patients in the third group have statistically higher reports of side effects associated with treatment than either the first, second, and fourth group.

### Example 47: Method of Treating Pain

Based on the inventor's experience, the following prophetic results are projected using controlled studies.

Four groups of female and male patients of age between 25 and 40 are admitted to treatment after having had been diagnosed with pain due to exercise related injuries. The first group is treated with a CBD, alpha-fenchome, and guaiacel containing lipid-based particle composition as disclosed herein topically. The second group is treated with a alpha-fenchome, guaiacel, para-cymene, and/or beta-camophyliene containing lipid-based particle composition as disclosed herein topically. The third group of patients is treated topically with a competitor liposomal CBD based composition made with CBD oil. The fourth group of patients is treated with a placebo topically. The first and second groups of patients experience recovery from pain faster than the third group and to a higher degree as measured by a self-evaluation. The results show statistically significant improvements in the first and second groups relative to either the third group or the fourth group.

The patients in the third group show statistically significant improvement over the placebo, but not to the degree achieved reported by the first or second groups. The patients in the third group have statistically higher reports of side effects associated with treatment than either the first, second, or fourth groups.

### Example 48: Method of Treating Insomnia

Based on the inventor's experience, the following prophetic results are projected using controlled studies.

Three groups of patients of age between 35 and 40 are treated for insomnia. The first group is treated with a CBN (10 mg/ml), CBD (2 mg/ml), and CBG (1 mg/ml) containing lipid-based particle composition as disclosed herein orally. The second group is treated with a CBN (10 mg/ml), CBD (2 mg/ml), CBG (1 mg/ml), valerian root, magnesium, GABA, melatonin, theanine, 5-HTP, tyrosine, zinc, and taurine containing lipid-based particle composition as disclosed herein orally. The third group of patients is treated orally with a competitor liposomal CBD oil-based composition. The fourth group of patients is treated with a placebo orally. The first and second groups of patients experience faster sleep time than the third group that is statistically significant. The second group also experiences faster sleep time than the first group. The patients in the third group show statistically significant improvement over the placebo, but not to the degree achieved reported by the first or second groups. The patients in the third group have statistically higher reports of side effects associated with treatment than either the first, second, or fourth groups.

### Example 49: Method of Treating Inflammation

Based on the inventor's experience, the following prophetic results are projected using controlled studies.

Three groups of patients of age between 25 and 55 are treated for inflammation. The first group is treated with an alpha-fenchome, alpha-terpineol, and bisabolol containing lipid-based particle composition as disclosed herein orally. The second group is treated with a CBD, alpha-fenchome, alpha-terpineol, and bisabolol containing lipid-based particle composition as disclosed herein orally. The third group of patients is treated orally with a competitor liposomal CBD oil-based composition. The fourth group of patients is treated with a placebo orally. Joint inflammation is measured after two weeks of twice a day administration of the formulations. After two weeks, the first and second groups of patients experience less inflammation than the third group at a statistically significant level. The second group also experiences les inflammation than the first group. The patients in the third group show statistically significant improvement over the placebo, but not to the degree achieved reported by the first or second groups. The patients in the third group have statistically higher reports of side effects associated with treatment than either the first, second, or fourth groups.

### Example 50: Method of Increasing Testosterone and Sperm Count in Adult Men

Based on the inventor's experience, the following prophetic results are projected using controlled studies.

Three groups of male patients of age between 25 and 55 are treated to increase testosterone and sperm count. The first group is treated with CBD, alpha-terpineol, alpha-finchome, and bisabolol containing lipid-based particle composition disclosed herein orally. The second group of patients are treated orally with a competitor CBD oil-based composition. The fourth group of patients are treated with a placebo orally. Prior to the start of the study, all patients were measured for basal levels of systemic testosterone and sperm count. Systemic levels of testosterone and sperm count were measured again after four weeks of twice a day administration of the formulations. After four weeks, the first group of patients had statistically significantly greater systemic levels of testosterone and significantly higher sperm count compared to their respective pre-study measurements, compared to groups two and three. The patients in the second and third group had no significant increase in systemic testosterone and sperm count compared to their respective baseline measurements.

### Example 51: Method of Treating Epilepsy

Based on the inventor's experience, the following prophetic results are projected using controlled studies.

Three groups of female and male patients of age between 25 and 40 are admitted to treatment after having had been diagnosed with epilepsy. The first group is treated with a CBD containing lipid-based particle composition as disclosed herein orally on a daily schedule. The second group of patients is treated orally on a daily schedule with a competitor liposomal CBD based composition made with CBD oil. The third group of patients is treated with a placebo orally on a daily schedule. The first group of patients experiences less seizures and symptoms of epilepsy than the second group or the third group. The results show statistically significant improvements in the first group relative to either the second group or the third group. The patients in the second group have statistically higher reports of side effects associated with treatment than either the first or the third group. The patients in the first group have electrical activity in the brain that is more similar to a non-epileptic patient than either the second or third groups. The electrical activity is measured using an electroencephalogram.

It is also found that a patient experiencing a seizure can be treated with a CBD containing lipid-based particle composition as disclosed herein orally to reduce the severity of seizure and/or shorten the duration (unlike comparator products containing CBD).

### Example 52: Method of Treating Diabetes

Based on the inventor's experience, the following prophetic results are projected using controlled studies.

Three groups of female and male patients of age between 25 and 40 are admitted to treatment after having had been diagnosed with diabetes. The first group is treated with a cannabinoid containing lipid-based particle composition as disclosed herein orally on a daily schedule. The second group of patients is treated orally on a daily schedule with a competitor liposomal cannabinoid based composition made with cannabinoid oil. The third group of patients is treated with a placebo orally on a daily schedule. The first group of patients experiences more stable blood glucose levels than the second group or the third group. The results show statistically significant improvements in the first group relative to either the second group or the third group. The patients in the second group have statistically higher reports of side effects associated with treatment than either the first or the third group. Compared to the second and third group, the first group also experiences lowering arterial inflammation due to the antioxidant properties of the cannabinoid, reducing neuropathic pain, a complication of diabetes, increased opening of blood vessels (which may reduce blood pressure over time and improve circulation), relief from muscle cramps, and relief from gastrointestinal pain and cramping.

### Example 53: Method of Treating Cancer

Based on the inventor's experience, the following prophetic results are projected using controlled studies.

Three groups of female and male patients of age between 25 and 55 are admitted to treatment after having had been diagnosed with a form of cancer (breast, colon, prostate, glioma, etc.). The first group is treated with a cannabinoid distillate containing lipid-based particle composition as disclosed herein intravenously on a daily schedule. The second group of patients is treated intravenously on a daily schedule with a competitor liposomal cannabinoid distillate based composition made with cannabinoid distillate oil. The third group of patients is treated with a placebo intravenously on a daily schedule. The first group of patients experiences slower tumor growth and, in some instances, cancer remission. The results show statistically significant improvements in the first group relative to either the second group or the third group. The patients in the second group have statistically higher reports of side effects associated with treatment than either the first or the third group.

1. A lipid-based particle composition, comprising:
a nanoparticle comprising:
a first therapeutic agent at a weight percent in the composition ranging from 1% to 20%;
a second therapeutic agent at a weight percent in the composition ranging from 1% to 20%;
a phosphatidylcholine at a weight percent in the composition ranging from 2.5% to 15%;
a sterol at a weight percent in the composition ranging from 0.5% to 5%; and
a medium chain triglyceride at a weight percent in the composition ranging from 2.5% to 15%; and
water at a weight percent in the composition ranging from 60% to about 95%;
wherein the nanoparticles have an average size ranging from about 20 nm to about 500 nm; and
wherein, upon exposure to sterilization conditions, the average size of the nanoparticles changes less than 2%.

2. The lipid-based particle composition of claim 1, wherein the composition comprises liposomes and/or an oil-in-water nano-emulsion.

3. The lipid-based particle composition of claim 1, wherein an appreciable amount of the nanoparticle composition does not settle and/or separate from the water upon standing for a period of at least about one month at room temperature.

4. The lipid-based particle composition of claim 1, wherein the composition is configured such that when concentrated to dryness to afford a powder formulation of nanoparticles, the nanoparticle powder can be reconstituted to provide the nanoparticle composition.

5. The lipid-based particle composition of claim 1, wherein the composition has a Tmax for CBD of less than 4.5 hours.

6. The lipid-based particle composition of claim 1, wherein, upon storage for a period of one month, the average size of the nanoparticles changes by less than about 20% and/or the polydispersity of the nanoparticles in the composition is less than or equal to 0.25.

7. (canceled)

8. The lipid-based particle composition of claim 1, wherein upon 90 days of storage at 25° C. and 60% relative humidity: the polydispersity of the nanoparticles changes by less than or equal to 100%; and/or the polydispersity of the nanoparticles changes by less than or equal to 0.1; and/or the D90 of the nanoparticles changes less than or equal to 20%.

9. (canceled)

10. The lipid-based particle composition of claim 1, wherein composition has a shelf life of greater than 18 months at 25° C. and 60% relative humidity.

11. (canceled)

12. The lipid-based particle composition of claim 1, wherein, when exposed to simulated gastric fluid at a pH of 1.6 for a period of at least 1 hour, the average size of the nanoparticles changes less than or equal to 10%;
and/or wherein, when exposed to simulated intestinal fluid at a pH of 6.5 for a period of at least 1 hour, the average size of the nanoparticles changes less than or equal to 10%; and/or
wherein the composition has a concentration max (Cmax) of 80 ng/ml after an oral dose of 15 mg/kg of CBD.

13. A lipid-based particle composition, comprising:
a particle comprising:
a first therapeutic agent at a weight percent in the composition ranging from 5% to 20%;
a second therapeutic agent at a weight percent in the composition ranging from 5% to 20%;
a phosphatidylcholine at a weight percent in the composition ranging from 35% to 60%;
a sterol at a weight percent in the composition ranging from 2.5% to 10%; and
a medium chain triglyceride at a weight percent in the composition ranging from 35% to 50%;
the lipid-based particle composition is provided as a dry powder;
wherein the powder is configured to be reconstituted in water to provide an aqueous solution;
wherein, upon reconstitution, nanoparticles within the aqueous solution have an average size ranging from about 20 nm to about 500 nm; and
wherein, upon exposure to sterilization conditions, the average size of the nanoparticles changes less than 2%.

14. The lipid-based particle of claim 13, wherein, upon reconstitution, nanoparticles within the aqueous solution have an average size ranging from about 75 nm to about 200 nm;
and/or wherein, when reconstituted and exposed to simulated gastric fluid at a pH of 1.6 for a period of at least 1 hour, the average size of the nanoparticles changes less than or equal to 10%; and/or
wherein, when reconstituted and exposed to simulated intestinal fluid at a pH of 6.5 for a period of at least 1 hour, the average size of the nanoparticles changes less than or equal to 10%.

15. The lipid-based particle composition of claim 1, wherein the sterilization conditions are selected from the group consisting of ozonation, UV treatment, and/or pasteurization.

16. The lipid-based particle composition of claim 1, further comprising a preservative, a flavoring agent, and/or one or more additional therapeutic agents.

17. (canceled)

18. The lipid-based particle composition of claim 1, wherein the sterol is cholesterol.

19.-21. (canceled)

22. The lipid-based particle composition of claim 1, wherein the first therapeutic and/or second therapeutic agent is a cannabinoid.

23. The lipid-based particle composition of claim 1, wherein the first therapeutic agent is CBD and the second therapeutic agent is a cannabinoid that is not CBD and/or is a terpene.

24. (canceled)

25. The lipid-based particle composition of claim 1, wherein the first therapeutic agent is CBD and the second therapeutic agent is selected from the group consisting of CBN, CBG, alpha-fenchome, alpha-terpineol, guaiacel, para-cymene, beta-camophyliene, phytol, bisabolol, and canephene.

26. (canceled)

27. A method of treating a patient in need of treatment comprising administering an effective amount of the lipid-based particle composition of claim 1 to the patient.

28. A method of manufacturing a nanoparticle composition of a therapeutic agent, comprising:
providing the therapeutic agent;
providing phosphatidylcholine;
providing a medium chain triglyceride;
mixing the medium chain triglyceride, phosphatidylcholine, and therapeutic agent to provide a solution; and
passing the solution through a microfluidizer to provide a lipid-based particle composition.

29. The method of claim 28, further comprising adding one or more sterols and/or water to the solution.

30. (canceled)
